(19) United States
(12) Patent Application Publication
(10) Pub. No.: US 2009/0136448 A1
(43) Pub. Date: May 28, 2009

(54) Antiviral 2-Carboxy-Thiophene Compounds

(76) Inventors: John Andrew Corfield, Hertfordshire (GB); Richard Martin Grimes, Hertfordshire (GB); David Harrison, Hertfordshire (GB); Charles David Hartley, Hertfordshire (GB); Peter David Howes, Hertfordshire (GB); Joelle Le, Hertfordshire (GB); Malcolm Lees Meeson, Hertfordshire (GB); Jacqueline Elizabeth Mordaunt, Hertfordshire (GB); Pritom Shah, Hertfordshire (GB); Martin John Slater, Hertfordshire (GB); Gemma Victoria White, Hertfordshire (GB)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 12/097,840

(22) PCT Filed: Dec. 20, 2006
(86) PCT No.: PCT/EP06/12442

§ 371 Date: Jun. 17, 2008

(30) Foreign Application Priority Data
Dec. 22, 2005 (GB) 0526197.9
Apr. 21, 2006 (GB) 0607978.4

(51) Int. Cl. A61K 38/21; C07D 491/02; A61K 31/4355; C07D 487/02; A61K 31/519; C07D 471/02; A61K 31/437; C07D 513/02; A61P 31/12; A61K 31/7056; A61K 31/429; C07D 263/54; A61K 31/423; A61K 31/5025; C07D 209/04; A61K 31/404

(52) U.S. Cl. 424/85.4; 546/115; 514/302; 544/281; 514/259.3; 546/121; 514/300; 548/154; 514/368; 548/224; 514/375; 544/236; 514/248; 548/469; 514/415; 514/43

(57) ABSTRACT

Anti-viral agents of compounds of Formula (I):

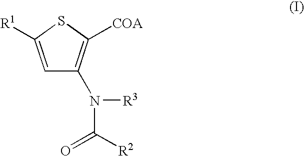

wherein A, R¹, R² and R³ are as defined in the specification, processes for their preparation and their use in HCV treatment are provided.

### FIELD OF THE INVENTION

The present invention relates to novel 2-carboxy thiophene derivatives useful as anti-viral agents. Specifically, the present invention involves novel inhibitors of Hepatitis C Virus (HCV) replication.

### BACKGROUND OF THE INVENTION

Infection with HCV is a major cause of human liver disease throughout the world. In the US, an estimated 4.5 million Americans are chronically infected with HCV. Although only 30% of acute infections are symptomatic, greater than 85% of infected individuals develop chronic, persistent infection. Treatment costs for HCV infection have been estimated at $5.46 billion for the US in 1997. Worldwide over 200 million people are estimated to be infected chronically. HCV infection is responsible for 40-60% of all chronic liver disease and 30% of all liver transplants. Chronic HCV infection accounts for 30% of all cirrhosis, end-stage liver disease, and liver cancer in the U. S. The CDC estimates that the number of deaths due to HCV will minimally increase to 38,000/year by the year 2010.

Due to the high degree of variability in the viral surface antigens, existence of multiple viral genotypes, and demonstrated specificity of immunity, the development of a successful vaccine in the near future is unlikely. Alpha-interferon (alone or in combination with ribavirin) has been widely used since its approval for treatment of chronic HCV infection. However, adverse side effects are commonly associated with this treatment: flu-like symptoms, leukopenia, thrombocytopenia, depression from interferon, as well as anemia induced by ribavirin (Lindsay, K. L. (1997) Hepatology 26 (suppl 1): 71S-77S). This therapy remains less effective against infections caused by HCV genotype 1 (which constitutes ˜75% of all HCV infections in the developed markets) compared to infections caused by the other 5 major HCV genotypes. Unfortunately, only ˜50-80% of the patients respond to this treatment (measured by a reduction in serum HCV RNA levels and normalization of liver enzymes) and, of responders, 50-70% relapse within 6 months of cessation of treatment. Recently, with the introduction of pegylated interferon (Peg-IFN), both initial and sustained response rates have improved substantially, and combination treatment of Peg-IFN with ribavirin constitutes the gold standard for therapy. However, the side effects associated with combination therapy and the impaired response in patients with genotype 1 present opportunities for improvement in the management of this disease.

First identified by molecular cloning in 1989 (Choo, Q-L et al (1989) Science 244:359-362), HCV is now widely accepted as the most common causative agent of post-transfusion non A, non-B hepatitis (NANBH) (Kuo, G et al (1989) Science 244:362-364). Due to its genome structure and sequence homology, this virus was assigned as a new genus in the Flaviviridae family. Like the other members of the Flaviviridae, such as flaviviruses (e.g. yellow fever virus and Dengue virus types 1-4) and pestiviruses (e.g. bovine viral diarrhea virus, border disease virus, and classic swine fever virus) (Choo, Q-L et al (1989) Science 244:359-362; Miller, R. H. and R. H. Purcell (1990) Proc. Natl. Acad. Sci. USA 87:2057-2061), HCV is an enveloped virus containing a single strand RNA molecule of positive polarity. The HCV genome is approximately 9.6 kilobases (kb) with a long, highly conserved, noncapped 5′ nontranslated region (NTR) of approximately 340 bases which functions as an internal ribosome entry site (IRES) (Wang C Y et al ‘An RNA pseudoknot is an essential structural element of the internal ribosome entry site located within the hepatitis C virus 5′ noncoding region’ RNA-A Publication of the RNA Society. 1(5): 526-537, 1995 Jul.). This element is followed by a region which encodes a single long open reading frame (ORF) encoding a polypeptide of ˜3000 amino acids comprising both the structural and nonstructural viral proteins.

Upon entry into the cytoplasm of the cell, this RNA is directly translated into a polypeptide of ˜3000 amino acids comprising both the structural and nonstructural viral proteins. This large polypeptide is subsequently processed into the individual structural and nonstructural proteins by a combination of host and virally-encoded proteinases (Rice, C. M. (1996) in B. N. Fields, D. M. Knipe and P. M. Howley (eds) Virology 2^{nd} Edition, p 931-960; Raven Press, N. Y.). Following the termination codon at the end of the long ORF, there is a 3′ NTR which roughly consists of three regions: an ˜40 base region which is poorly conserved among various genotypes, a variable length poly(U)/polypyrimidine tract, and a highly conserved 98 base element also called the “3′ X-tail” (Kolykhalov, A. et al (1996) J. Virology 70:3363-3371; Tanaka, T. et al (1995) Biochem Biophys. Res. Commun. 215:744-749; Tanaka, T. et al (1996) J. Virology 70:3307-3312; Yamada, N. et al (1996) Virology 223:255-261). The 3′ NTR is predicted to form a stable secondary structure which is essential for HCV growth in chimps and is believed to function in the initiation and regulation of viral RNA replication.

The NS5B protein (591 amino acids, 65 kDa) of HCV (Behrens, S. E. et al (1996) EMBO J. 15:12-22), encodes an RNA-dependent RNA polymerase (RdRp) activity and contains canonical motifs present in other RNA viral polymerases. The NS5B protein is fairly well conserved both intra-typically (˜95-98% amino acid (aa) identity across 1b isolates) and inter-typically (˜85% aa identity between genotype 1a and 1b isolates). The essentiality of the HCV NS5B RdRp activity for the generation of infectious progeny virions has been formally proven in chimpanzees (A. A. Kolykhalov et al. (2000) Journal of Virology, 74(4): 2046-2051). Thus, inhibition of NS5B RdRp activity (inhibition of RNA replication) is predicted to be useful to treat HCV infection.

Although the predominant HCV genotype worldwide is genotype 1, this itself has two main subtypes, denoted 1a and 1b. As seen from entries into the Los Alamos HCV database (www.hcv.lanl.gov) (Table 1) there are regional differences in the distribution of these subtypes: while genotype 1a is most abundant in the United States, the majority of sequences in Europe and Japan are from genotype 1b.

TABLE 1

| % of sequences | | | | |
|---|---|---|---|---|
| in the database | World | USA | Europe | Japan |
| Genotype 1 | 71.8 | 87.8 | 75.9 | 80.2 |
| Genotype 1a | 28.4 | 66.4 | 21.7 | 1.6 |
| Genotype 1b | 43.4 | 21.4 | 54.2 | 78.6 |

Based on the foregoing, there exists a significant need to identify synthetic or biological compounds for their ability to inhibit replication of both genotype 1a and genotype 1b of HCV.

PCT publication number WO2002/100851 generically discloses certain compounds, including certain 2-carboxy thiophene compounds, having HCV inhibitory activity. The data provided relates to an HCV polymerase assay utilising the 1b genotype. The compounds disclosed have the formula (I), herein disclosed as formula (A)

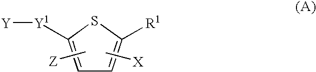

wherein
X is chosen from —N(R³)M(R²) or -JN(R²)(R³);
M is chosen from —SO₂—, —SO—, —S—, —C(O)—, —C(S)—, —CH₂C(O)N(R⁴)—, —CH₂C(S)N(R¹⁵)—, —CH(R¹⁵)—, —C(═N(R⁸))—, or a bond;
R⁴ is C₁₋₆alkyl;
R⁸ is chosen from H, C₁₋₁₂alkyl, C₂₋₁₂alkenyl, C₂₋₁₂alkynyl, C₆₋₁₄aryl, C₃₋₁₂heterocycle, C₃₋₁₂heteroaralkyl, C₆₋₁₆aralkyl;
R¹⁵ is chosen from H or C₁₋₆alkyl;
J is chosen from —C(W)—, —C(R⁶)—, —S—, —S(O)—, or —SO₂—;
W is chosen from O, S or NR⁷;
R⁷ is chosen from H, C₁₋₁₂alkyl, C₂₋₁₂alkenyl, C₂₋₁₂alkynyl, C₆₋₁₄aryl, C₃₋₁₂heterocycle, C₃₋₁₂heteroaralkyl, C₆₋₁₆aralkyl;
R⁶ is chosen from H, C₁₋₁₂alkyl, C₆₋₁₄aryl, or C₆₋₁₆aralkyl;
Y¹ is chosen from a bond, C₁₋₆alkyl, C₂₋₆alkenyl, C₂₋₆alkynyl;
Y is chosen from COOR¹⁶, COCOOR⁵, P(O)OR^{a}OR^{b}, S(O)OR⁵, S(O)₂OR⁵, tetrazole, CON(R⁹)CH(R⁵)COOR⁵, CONR¹⁰R¹¹, CON(R⁹)—SO₂—R⁵, CONR⁹OH, or halogen;
R⁹, R⁵, R¹⁰ and R¹¹ are each independently chosen from H, C₁₋₁₂alkyl, C₂₋₁₂alkenyl, C₂₋₁₂alkynyl, C₃₋₁₂heterocycle, C₃₋₁₈heteroaralkyl, C₆₋₁₈aralkyl;
or R¹⁰ and R¹¹ are taken together with the nitrogen to form a 3 to 10 membered heterocycle;
R^{a} and R^{b} are each independently chosen from H, C₁₋₁₂alkyl, C₂₋₁₂alkenyl, C₂₋₁₂alkynyl, C₆₋₁₄aryl, C₃₋₁₂heterocycle, C₃₋₁₈heteroaralkyl, C₆₋₁₈aralkyl;
or R^{a} and R^{b} are taken together with the oxygens to form a 5 to 10 membered heterocycle;
R¹⁶ is chosen from H, C₁₋₂alkyl, C₂₋₁₂alkenyl, C₂₋₁₂alkynyl, C₆₋₁₄aryl, C₃₋₁₂heterocycle, C₃₋₁₈heteroaralkyl, C₆₋₁₈aralkyl; provided that R¹⁶ is other than methyl or ethyl;
R¹ is chosen from C₁₋₁₂alkyl, C₂₋₁₂alkenyl, C₂₋₁₂alkynyl, C₆₋₁₄aryl, C₃₋₁₂heterocycle, C₃₋₁₈heteroaralkyl, C₆₋₁₈aralkyl;
R² is chosen from C₁₋₁₂alkyl, C₂₋₁₂alkynyl, C₆₋₁₄aryl, C₃₋₁₂heterocycle, C₃₋₁₈heteroaralkyl, C₆₋₁₈aralkyl;
R³ is chosen from H, C₁₋₁₂alkyl, C₂₋₁₂alkenyl, C₂₋₁₂alkynyl, C₆₋₁₄aryl, C₃₋₁₂heterocycle, C₃₋₁₈heteroaralkyl, C₆₋₁₈aralkyl;
Z is chosen from H, halogen, or C₁₋₆alkyl.

Surprisingly, it has now been found that compounds according to the present invention, generically disclosed in WO2002/100851, and having a specific substitution pattern, exhibit improved properties over those compounds specifically disclosed in WO2002/100851.

### SUMMARY OF THE INVENTION

The present invention involves novel 2-carboxy thiophene compounds represented hereinbelow, pharmaceutical compositions comprising such compounds and use of the compounds in treating viral infection, especially HCV infection.

### DETAILED DESCRIPTION OF THE INVENTION

The present invention provides a compound of Formula (I):

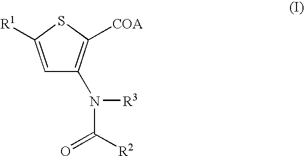

wherein:
A represents hydroxy;
R¹ represents —R^{X}—R^{Y};
R^{X} represents phenyl (optionally substituted by halo, methyl, ethyl, methoxy or trifluoromethyl) or 5 or 6-membered heteroaryl bonded through a ring carbon atom to the carbon atom of the thiophene; in the case of 6-membered heteroaryl there not being a ring nitrogen ortho to the attachment point to the thiophene;

R^{Y} represents 8, 9 or 10-membered heteroaryl, bonded such that when R^{X} is phenyl, R^{Y} is in the para-position; the heteroaryl not being imidazo[1,2-a]pyridin-6-yl;

R² represents C₅₋₇cycloalkyl optionally substituted by one or more substitutents selected from —C₁₋₆alkyl or —OR^{A};
R³ represents linear or branched —C₁₋₆alkyl (unsubstituted), or linear or branched —C₁₋₆alkyl substituted with C₃₋₆cycloalkyl;
R^{A} represents hydrogen or —C₁₋₆alkyl;
or a salt, solvate or ester thereof.

The compounds of the present invention exhibit improved potency against the replication of HCV (1a and 1b genotypes), and therefore have the potential to achieve greater efficacy in man. High potency in both genotypes is considered to be advantageous.

There is provided as a further aspect of the present invention a compound of Formula (I) or a pharmaceutically acceptable salt, solvate or ester thereof for use in human or veterinary medical therapy, particularly in the treatment or prophylaxis of viral infection, particularly flavivirus infection, for example HCV infection.

It will be appreciated that reference herein to therapy and/or treatment includes, but is not limited to prevention, retardation, prophylaxis, therapy and cure of the disease. It will further be appreciated that references herein to treatment or prophylaxis of HCV infection include treatment or prophylaxis of HCV-associated disease such as liver fibrosis, cirrhosis and hepatocellular carcinoma.

In a further or alternative aspect there is provided a method for the treatment of a human or animal subject with viral infection, particularly HCV infection, which method comprises administering to said human or animal subject an effective amount of a compound of Formula (I) or a pharmaceutically acceptable salt, solvate or ester thereof.

According to another aspect of the invention, there is provided the use of a compound of Formula (I) or a pharmaceutically acceptable salt, solvate or ester thereof in the manufacture of a medicament for the treatment and/or prophylaxis of viral infection, particularly HCV infection.

It will be appreciated that the compounds of the present invention may contain one or more asymmetric carbon atoms and may exist in racemic, diastereoisomeric, and optically active forms. All of these racemic compounds, enantiomers and diastereoisomers are contemplated to be within the scope of the present invention.

In one aspect, R^{X} represents phenyl optionally substituted by halo, methyl, methoxy or trifluoromethyl. In a further aspect, R^{X} represents unsubstituted phenyl or halo-substituted phenyl. In a further aspect, R^{X} represents unsubstituted phenyl or 3-chlorophenyl. In a further aspect, R^{X} represents unsubstituted phenyl.

In a further aspect, R^{X} represents unsubstituted thienyl, unsubstituted furanyl, or unsubstituted pyridinyl (wherein the ring nitrogen is not in the ortho position relative to the thiophene attachment). In a further aspect, R^{X} represents unsubstituted thienyl attached to the rest of the molecule via its 2- and 5-positions, unsubstituted furanyl attached to the thiophene via its 3-position and to R^{Y} via its 5-position, or unsubstituted pyridinyl (wherein the ring nitrogen is not in the ortho position relative to the thiophene attachment) attached to the thiophene via its 5-position and to R^{Y} via its 2-position.

In one aspect, R^{Y} represents 8- or 9-membered heteroaryl. In a further aspect, R^{Y} represents furo[3,2-b]pyridin-2-yl, pyrazolo[1,5-a]pyrimidin-2-yl, imidazo[1,2-a]pyridin-2-yl, imidazo[2,1-b][1,3]thiazol-6-yl, 7-amino-5-methylpyrazolo-[1,5-a]pyrimidin-2-yl, 5-methylpyrazolo-[1,5-a]pyrimidin-2-yl, 7-aminopyrazolo[1,5-a]pyrimidin-2-yl, [1,3]oxazolo[4,5-b]pyridin-2-yl, furo[2,3-b]pyridin-5-yl, 5-amino-1,3-benzoxazol-2-yl, [1,3]oxazolo[5,4-b]pyridin-2-yl, furo[3,2-c]pyridin-2-yl, 4-amino-1,3-benzoxazol-2-yl, pyrazolo[1,5-a]pyrimidin-5-yl, 7-hydroxy-1-benzofuran-2-yl, 7-hydroxy-1,3-benzoxazol-2-yl, pyrazolo[1,5-b]pyridazin-2-yl, 6-aminoimidazo[1,2-a]pyridin-2-yl, 1H-benzimidazol-5-yl, 5-amino-1-benzofuran-2-yl, 6-amino-1-benzofuran-2-yl, 6-amino-1,3-benzoxazol-2-yl, 1,3-benzoxazol-2-yl, 1H-indol-5-yl or 1H-indol-6-yl.

In a further aspect, R^{Y} represents furo[3,2-b]pyridin-2-yl, pyrazolo[1,5-a]pyrimidin-2-yl, [1,3]oxazolo[4,5-b]pyridin-2-yl, 5-methylpyrazolo[1,5-a]pyrimidin-2-yl, imidazo[2,1-b][1,3]thiazol-6-yl, 7-amino-5-methylpyrazolo[1,5-a]pyrimidin-2-yl, 7-hydroxy-1-benzofuran-2-yl or 7-aminopyrazolo[1,5-a]pyrimidin-2-yl.

In a further aspect, R^{Y} represents furo[3,2-b]pyridin-2-yl, pyrazolo[1,5-a]pyrimidin-2-yl, imidazo[1,2-a]pyridin-2-yl, 4-imidazo[2,1-b][1,3]thiazol-6-yl, 7-amino-5-methylpyrazolo[1,5-a]pyrimidin-2-yl, 5-methylpyrazolo-[1,5-a]pyrimidin-2-yl, 7-aminopyrazolo[1,5-a]pyrimidin-2-yl, [1,3]oxazolo[4,5-b]pyridin-2-yl, furo[2,3-b]pyridin-5-yl, 5-amino-1,3-benzoxazol-2-yl, [1,3]oxazolo[5,4-b]pyridin-2-yl or furo[3,2-c]pyridin-2-yl.

In a further aspect, R^{Y} represents furo[3,2-b]pyridin-2-yl, pyrazolo[1,5-a]pyrimidin-2-yl or imidazo[1,2-a]pyridin-2-yl.

In one aspect, R² represents C₆cycloalkyl optionally substituted by one or more C₁₋₄alkyl substituents. In a further aspect, R² represents C₆cycloalkyl optionally substituted by one or more C₁₋₄alkyl substituents substituted with fluoro. In a further aspect, R² represents trans-4-methylcyclohexyl or trans-4-trifluoromethylcyclohexyl. In a further aspect, R² represents trans-4-methylcyclohexyl.

In one aspect, R³ represents linear or branched —C₁₋₆alkyl (unsubstituted), for example 1-methylethyl or ethyl. In a further aspect, R³ represents linear or branched —C₁₋₆alkyl substituted with C₃₋₆cycloalkyl, for example cyclopropylmethyl. In a further aspect, R³ represents 1-methylethyl.

It is to be understood that the present invention covers all combinations of aspects and further aspects described herein.

As used herein, “acetyl” refers to —C(O)CH₃.

As used herein unless otherwise specified, “alkyl” refers to an optionally substituted hydrocarbon group. The alkyl hydrocarbon group may be linear, branched or cyclic, saturated or unsaturated. Where the alkyl group is linear or branched, examples of such groups include methyl, ethyl, n-propyl, 1-methylethyl(isopropyl), n-butyl, isobutyl, sec-butyl, tert-butyl, n-pentyl, isopentyl, neopentyl or hexyl and the like. Where the alkyl hydrocarbon group is unsaturated, it will be understood that there will be a minimum of 2 carbon atoms in the group, for example an alkenyl or alkynyl group. Where the alkyl hydrocarbon group is cyclic, it will be understood that there will be a minimum of 3 carbon atoms in the group. In one aspect, alkyl moieties are saturated. In one aspect, alkyl moieties are —C₁₋₄alkyl. Unless otherwise stated, optional substituents include —C₁₋₆alkyl (unsubstituted), ═CH(CH₂)ₜH, fluoro, —CF₃, —OR^{E}, —SR^{E}, —C(O)NR^{B}R^{C}, —C(O)R^{D}, —CO₂H, —CO₂R^{D}, —NR^{B}R^{C}, —NR^{A}C(O)R^{D}, —NR^{A}CO₂R^{D}, —NR^{A}C(O)NR^{F}R^{G}, —SO₂NR^{F}R^{G}, —SO₂R^{D}, nitro, cyano, oxo, aryl, heteroaryl and heterocyclyl.

As used herein, the term “alkenyl” refers to a linear or branched hydrocarbon group containing one or more carbon-carbon double bonds. In one aspect the alkenyl group has from 2 to 6 carbon atoms. Examples of such groups include ethenyl, propenyl, butenyl, pentenyl or hexenyl and the like.

As used herein, the term “alkynyl” refers to a linear or branched hydrocarbon group containing one or more carbon-carbon triple bonds. In one aspect the alkynyl group has from 2 to 6 carbon atoms. Examples of such groups include ethynyl, propynyl, butynyl, pentynyl or hexynyl and the like.

As used herein unless otherwise specified, “cycloalkyl” refers to an optionally substituted, cyclic hydrocarbon group. The hydrocarbon group may be saturated or unsaturated, monocyclic or bridged bicyclic. Where the cycloalkyl group is saturated, examples of such groups include cyclopropyl, cyclobutyl, cyclopentyl, cyclohexyl, cycloheptyl or cyclooctyl and the like. Where the cycloalkyl group is unsaturated, examples of such groups include cyclobutenyl, cyclopentenyl, cyclohexenyl, cycloheptenyl or cyclooctenyl and the like. In one aspect, the cycloalkyl group has from 5 to 7 carbon atoms. In one aspect, cycloalkyl moieties are cyclohexenyl, cyclopentenyl and cyclohexyl. Unless otherwise stated, the cycloalkyl group may be substituted by one or more optional substituents including —C₁₋₆alkyl (unsubstituted), ═CH(CH₂)ₜH, fluoro, —CF₃, —OR^{E}, —SR^{E}, C(O)NR^{B}R^{C}, —C(O)R^{D}, —CO₂H, —CO₂R^{D}, —NR^{B}R^{C}, —NR^{A}C(O)R^{D}, —NR^{A}CO₂R^{D}, —NR^{A}C(O)NR^{F}R^{G}, —SO₂NR^{F}R^{G}, —SO₂R^{D}, nitro, cyano, oxo, phenyl and heterocyclyl.

As used herein, the term “alkoxy” refers to an —O-alkyl group wherein alkyl is as defined herein. Examples of such groups include methoxy, ethoxy, propoxy, butoxy, pentoxy or hexoxy and the like.

As used herein, “aryl” refers to an optionally substituted aromatic group with at least one ring having a conjugated pi-electron system, containing up to two conjugated or fused ring systems. “Aryl” includes carbocyclic aryl and biaryl groups, all of which may be optionally substituted. In one aspect, “aryl” moieties contain 6-10 carbon atoms. In one aspect, “aryl” moieties are unsubstituted, monosubstituted, disubstituted or trisubstituted phenyl. In one aspect, unless otherwise stated, “aryl” substituents are selected from the group consisting of —C₁₋₆alkyl, halo, —OR^{E}, —SR^{E}, —C(O)NR^{B}R^{C}, —C(O)R^{D}, —CO₂H, —CO₂R^{D}, —NR^{B}R^{C}, —NR^{A}C(O)R^{D}, —NR^{A}CO₂R^{D}, —NR^{A}C(O)NR^{F}R^{G}, —SO₂NR^{F}R^{G}, —SO₂R^{D}, nitro, cyano, heterocyclyl, —CF₃, —OCF₃ and phenyl.

As used herein, “carbonyl” refers to —C(O)—.

As used herein, “cyano” refers to —CN.

As used herein, “halogen” or “halo” refer to a fluorine, chlorine, bromine or iodine atom. References to “fluoro”, “chloro”, “bromo” or “iodo” should be construed accordingly.

As used herein, unless otherwise specified, “heteroaryl” refers to an optionally substituted, 5, 6, 8, 9 or 10 membered, aromatic group comprising one to four heteroatoms selected from N, O and S, with at least one ring having a conjugated pi-electron system, containing up to two conjugated or fused ring systems. In one aspect, “heteroaryl” moieties are unsubstituted, monosubstituted, disubstituted or trisubstituted (where applicable) pyridine, pyrazine, thiazole, thiophene, oxadiazole, oxazole, pyrimidine, pyridazine, benzodioxole, benzofuran, benzodioxin, indole, benzimidazole, benzofuran, indole, indazole, isoindole, benzothiophene, benzothiazole, benzoxazole, benzisoxazole, benzisothiazole, benzotriazole, furopyridine, furopyrimidine, furopyridazine, furopyrazine, furotriazine, pyrrolopyridine, pyrrolopyrimidine, pyrrolopyridazine, pyrrolopyrazine, pyrrolotriazine, thienopyridine, thienopyrimidine, thienopyridazine, thienopyrazine, thienotriazine, thiazolopyridine, thiazolopyrimidine, thiazolopyridazine, thiazolopyrazine, thiazolotriazine, oxazolopyridine, oxazolopyrimidine, oxazolopyridazine, oxazolopyrazine, oxazolotriazine, imidazopyridine, imidazopyrimidine, imidazopyridazine, imidazopyrazine, imidazotriazine, pyrazolopyridine, pyrazolopyrimidine, pyrazolopyridazine, pyrazolopyrazine, pyrazolotriazine, triazolopyridine, triazolopyrimidine, triazolopyridazine, triazolopyrazine, quinoline, naphthyridine, quinoxaline, quinazoline, isoquinoline, cinnoline, pyridopyridazine, pyridopyrimidine, pyridopyrazine, pyrazinopyrazine, pteridine, pyrazinopyridazine, pyrimidopyridazine, pyrimidopyrimidine, imidazothiazole, thiazolooxazole. All isomers of the above heteroaryls are within the scope of this invention. Each heteroaryl group may be attached at any ring carbon or may be attached through nitrogen when the nitrogen is part of a 5-membered ring. In one aspect, unless otherwise stated, “heteroaryl” substituents are selected from the group consisting of —C₁₋₆alkyl, halo, —OR^{E}, —SR^{E}, —C(O)NR^{B}R^{C}, —C(O)R^{D}, —CO₂R^{D}, —NR^{B}R^{C}, —NR^{A}C(O)R^{D}, —NR^{A}CO₂R^{D}, —NR^{A}C(O)NR^{F}R^{G}, —SO₂NR^{F}R^{G}, —SO₂R^{D}, oxo, nitro, cyano, heterocyclyl, —CF₃ and phenyl.

As used herein, “heterocyclic” and “heterocyclyl” refer to an optionally substituted, 5 or 6 membered, saturated or partially saturated, cyclic group containing 1 or 2 heteroatoms selected from N, optionally substituted by hydrogen, —C₁₋₆alkyl, —C(O)R^{D}, —C(O)NR^{B}R^{C}, —C(O)OH, —SO₂R^{D}, aryl or heteroaryl; O; and S, optionally substituted by one or two oxygen atoms. Ring carbon atoms may be optionally substituted by —C₁₋₆alkyl, —OR^{A}, —C(O)R^{D}, or —SO₂R^{D}. In one aspect, unless otherwise stated, “heterocyclic” moieties are unsubstituted or monosubstituted tetrahydro-2H-pyran-4-yl, piperidinyl and tetrahydrofuran-3-yl.

As used herein, “nitro” refers to —NO₂.

As used herein, “oxo” refers to ═O.

As used herein, “Et” refers to “ethyl”, “iPr” refers to “isopropyl”, “Me” refers to “methyl”, “OBn” refers to “benzyloxy”, and “Ph” refers to “phenyl”.

R^{A} represents hydrogen or —C₁₋₆alkyl.

R^{B} and R^{C} independently represent hydrogen, —C₁₋₆alkyl, aryl, heterocyclyl or heteroaryl; or R^{B} and R^{C} together with the nitrogen atom to which they are attached form a 5 or 6 membered saturated cyclic group.

R^{D} is selected from the group consisting of —C₁₋₆alkyl, aryl, heterocyclyl, heteroaryl, arylalkyl, and heteroarylalkyl.

R^{E} represents hydrogen, —C₁₋₆alkyl, arylalkyl, heteroarylalkyl, aryl, heterocyclyl or heteroaryl.

R^{F} and R^{G} are independently selected from the group consisting of hydrogen, —C₁₋₆alkyl, aryl, heteroaryl, arylalkyl, and heteroarylalkyl; or R^{F} and R^{G} together with the nitrogen atom to which they are attached form a 5 or 6 membered saturated cyclic group.

In one aspect, compounds useful in the present invention may be chosen from compounds of Formula (I) selected from the group consisting of:
5-(4-Furo[3,2-b]pyridin-2-ylphenyl)-3-[[(trans-4-methylcyclohexyl)carbonyl](1-methylethyl)amino]-2-thiophenecarboxylic acid;
3-[[(trans-4-Methylcyclohexyl)carbonyl](1-methylethyl)amino]-5-(4-pyrazolo[1,5-a]pyrimidin-2-ylphenyl)-2-thiophenecarboxylic acid;
5-(4-Imidazo[1,2-a]pyridin-2-ylphenyl)-3-[[(trans-4-methylcyclohexyl)carbonyl](1-methylethyl)amino]-2-thiophenecarboxylic acid;
5-(4-Imidazo[2,1-b][1,3]thiazol-6-ylphenyl)-3-[[(trans-4-methylcyclohexyl)carbonyl](1-methylethyl)amino]-2-thiophenecarboxylic acid;
5-[4-(7-Amino-5-methylpyrazolo[1,5-a]pyrimidin-2-yl)phenyl]-3-[[(trans-4-methyl-cyclohexyl)carbonyl](1-methylethyl)amino]-2-thiophenecarboxylic acid;
3-[[(trans-4-Methylcyclohexyl)carbonyl] (1-methylethyl)amino]-5[4-(5-methylpyrazolo-[1,5-a]pyrimidin-2-yl)phenyl]-2-thiophenecarboxylic acid;
5-[4-(7-Aminopyrazolo[1,5-a]pyrimidin-2-yl)phenyl]-3-[[(trans-4-methylcyclohexyl)-carbonyl](1-methylethyl)amino]-2-thiophenecarboxylic acid;
3-[[(4-Methylcyclohexyl)carbonyl](1-methylethyl)amino]-5-(4-[1,3]oxazolo[4,5-b]pyridin-2-ylphenyl)-2-thiophenecarboxylic acid;
5-(4-furo[2,3-b]pyridin-5-ylphenyl)-3-[[(trans-4-methylcyclohexyl)carbonyl](1-methylethyl)amino]-2-thiophenecarboxylic acid;
5-[4-(5-Amino-1,3-benzoxazol-2-yl)phenyl]-3-[[(trans-4-methylcyclohexyl)carbonyl](1-methylethyl)amino]-2-thiophenecarboxylic acid;
3-[[(4-Methylcyclohexyl)carbonyl](1-methylethyl)amino]-5-(4-[1,3]oxazolo[5,4-b]pyridin-2-ylphenyl)-2-thiophenecarboxylic acid;
5-(4-Furo[3,2-c]pyridin-2-ylphenyl)-3-[[(trans-4-methylcyclohexyl)carbonyl](1-methylethyl)-amino]-2-thiophenecarboxylic acid;
5-(4-Imidazo[1,2-a]pyridin-2-ylphenyl)-3-((1-methylethyl){[trans-4-(trifluoromethyl)cyclohexyl]carbonyl}amino)-2-thiophenecarboxylic acid;
3-((1-Methylethyl){[trans-4-(trifluoromethyl)cyclohexyl]carbonyl}amino)-5-(4-pyrazolo[1,5-a]pyrimidin-2-ylphenyl)-2-thiophenecarboxylic acid;
5-[4-(4-Amino-1,3-benzoxazol-2-yl)phenyl]-3-[[(trans-4-methylcyclohexyl)carbonyl](1-methylethyl)amino]-2-thiophenecarboxylic acid;
3-[[(trans-4-Methylcyclohexyl)carbonyl](1-methylethyl)amino]-5-(4-pyrazolo[1,5-a]pyrimidin-5-ylphenyl)-2-thiophenecarboxylic acid;
5-(6-Furo[3,2-b]pyridin-2-yl-3-pyridinyl)-3-[[(trans-4-methylcyclohexyl)carbonyl](1-methylethyl)amino]-2-thiophenecarboxylic acid;
5-[4-(7-Hydroxy-1-benzofuran-2-yl)phenyl]-3-[[(trans-4-methylcyclohexyl)carbonyl](1-methylethyl)amino]-2-thiophenecarboxylic acid;
5-[4-(7-Hydroxy-1,3-benzoxazol-2-yl)phenyl]-3-[[(trans-4-methylcyclohexyl)carbonyl](1-methylethyl)amino]-2-thiophenecarboxylic acid;
5-(4-Imidazo[2,1-b][1,3]thiazol-6-ylphenyl)-3-((1-methylethyl){[trans-4-(trifluoromethyl)cyclohexyl]carbonyl}amino)-2-thiophenecarboxylic acid;
5-(4-Furo[3,2-b]pyridin-2-ylphenyl)-3-((1-methylethyl){[trans-4-(trifluoromethyl)cyclohexyl]carbonyl}amino)-2-thiophenecarboxylic acid;
5-[4-(6-Amino-1,3-benzoxazol-2-yl)phenyl]-3-[[(trans-4-methylcyclohexyl)carbonyl](1-methylethyl)amino]-2-thiophenecarboxylic acid;
3-[[(trans-4-Methylcyclohexyl)carbonyl](1-methylethyl)amino]-5-(4-pyrazolo[1,5-b]pyridazin-2-ylphenyl)-2-thiophenecarboxylic acid;
3-((1-Methylethyl){[trans-4-(trifluoromethyl)cyclohexyl]carbonyl}amino)-5-(4-[1,3]oxazolo[4,5-b]pyridin-2-ylphenyl)-2-thiophenecarboxylic acid;
3-((1-Methylethyl){[trans-4-(trifluoromethyl)cyclohexyl]carbonyl}amino)-5-[4-(5-methylpyrazolo[1,5-a]pyrimidin-2-yl)phenyl]-2-thiophenecarboxylic acid;
5-[4-(6-Aminoimidazo[1,2-a]pyridin-2-yl)phenyl]-3-[[(trans-4-methylcyclohexyl)carbonyl](1-methylethyl)amino]-2-thiophenecarboxylic acid;
5-[4-(1H-Benzimidazol-5-yl)phenyl]-3-[[(trans-4-methylcyclohexyl)carbonyl] (1-methylethyl)amino]-2-thiophenecarboxylic acid;
5-[4-(5-Amino-1-benzofuran-2-yl)phenyl]-3-[[(trans-4-methylcyclohexyl)carbonyl](1-methylethyl)amino]-2-thiophenecarboxylic acid;
5-[4-(6-Amino-1-benzofuran-2-yl)phenyl]-3-[[(trans-4-methylcyclohexyl)carbonyl](1-methylethyl)amino]-2-thiophenecarboxylic acid;
5-[5-(1,3-Benzoxazol-2-yl)-3-furanyl]-3-[[(trans-4-methylcyclohexyl)carbonyl](1-methylethyl)amino]-2-thiophenecarboxylic acid;
5-(6-Imidazo[2,1-b][1,3]thiazol-6-yl-3-pyridinyl)-3-[[(trans-4-methylcyclohexyl)carbonyl](1-methylethyl)amino]-2-thiophenecarboxylic acid;
5-[4-(7-Aminopyrazolo[1,5-a]pyrimidin-2-yl)-3-chlorophenyl]-3-[[(trans-4-methylcyclohexyl)carbonyl](1-methylethyl)amino]-2-thiophenecarboxylic acid;
3-[[(trans-4-Methylcyclohexyl)carbonyl](1-methylethyl)amino]-5-(6-pyrazolo[1,5-a]pyrimidin-2-yl-3-pyridinyl)-2-thiophenecarboxylic acid;
3-{(Cyclopropylmethyl)[(trans-4-methylcyclohexyl)carbonyl]amino}-5-(4-pyrazolo[1,5-a]pyrimidin-2-ylphenyl)-2-thiophenecarboxylic acid;
4-[[(trans-4-Methylcyclohexyl)carbonyl](1-methylethyl)amino]-5′-pyrazolo[1,5-a]pyrimidin-2-yl-2,2′-bithiophene-5-carboxylic acid;
3-[{[(1S,2R,4S)-2-Hydroxy-4-methylcyclohexyl]carbonyl}(1-methylethyl)amino]-5-(4-pyrazolo[1,5-a]pyrimidin-2-ylphenyl)-2-thiophenecarboxylic acid;
5-[4-(1H-Indol-5-yl)phenyl]-3-[[(trans-4-methylcyclohexyl)carbonyl] (1-methylethyl)amino]-2-thiophenecarboxylic acid;
5-[4-(1H-Indol-6-yl)phenyl]-3-[[(trans-4-methylcyclohexyl)carbonyl](1-methylethyl)amino]-2-thiophenecarboxylic acid;
5′-Furo[3,2-b]pyridin-2-yl-4-[[(trans-4-methylcyclohexyl)carbonyl](1-methylethyl)amino]-2,2′-bithiophene-5-carboxylic acid;
3-[[(trans-4-Methylcyclohexyl)carbonyl](1-methylethyl)amino]-5-(6-[1,3]oxazolo[4,5-b]pyridin-2-yl-3-pyridinyl)-2-thiophenecarboxylic acid;
   and salts, solvates and esters, and individual enantiomers thereof where appropriate.

In one aspect, salts of compounds useful in the present invention may be chosen from the group consisting of:
Sodium 5-(4-furo[3,2-c]pyridin-2-ylphenyl)-3-[[(trans-4-methylcyclohexyl)carbonyl](1-methylethyl)amino]-2-thiophenecarboxylate;
Sodium 3-[[(trans-4-methylcyclohexyl)carbonyl](1-methylethyl)amino]-5-(4-pyrazolo[1,5-a]pyrimidin-2-ylphenyl)-2-thiophenecarboxylate;
Ammonium 3-[[(trans-4-methylcyclohexyl)carbonyl](1-methylethyl)amino]-5-(4-pyrazolo[1,5-a]pyrimidin-2-ylphenyl)-2-thiophenecarboxylate;
Ammonium 3-{ethyl[(trans-4-methylcyclohexyl)carbonyl]amino}-5-(4-pyrazolo[1,5-a]pyrimidin-2-ylphenyl)-2-thiophenecarboxylate;
Potassium 5-(4-furo[3,2-b]pyridin-2-ylphenyl)-3-[[(trans-4-methylcyclohexyl)carbonyl](1-methylethyl)amino]-2-thiophenecarboxylate; and
Trisamine 5-(4-furo[3,2-b]pyridin-2-ylphenyl)-3-[[(trans-4-methylcyclohexyl)carbonyl](1-methylethyl)amino]-2-thiophenecarboxylate.

Also included in the present invention are pharmaceutically acceptable salt complexes. The present invention also covers the pharmaceutically acceptable salts of the compounds of Formula (I). Suitable pharmaceutically acceptable salts of the compounds of Formula (I) include acid salts, for example sodium, potassium, calcium, magnesium and tetraalkylammonium and the like, or mono- or di-basic salts with the appropriate acid for example organic carboxylic acids such as acetic, lactic, tartaric, malic, isethionic, lactobionic and succinic acids; organic sulfonic acids such as methanesulfonic, ethanesulfonic, benzenesulfonic and p-toluenesulfonic acids and inorganic acids such as hydrochloric, sulfuric, phosphoric and sulfamic acids and the like.

The present invention also relates to solvates of the compounds of Formula (I), for example hydrates.

The present invention also relates to pharmaceutically acceptable esters of the compounds of Formula (I), for example carboxylic acid esters —COOR, in which R is selected from straight or branched chain alkyl, for example n-propyl, n-butyl, alkoxyalkyl (e.g. methoxymethyl), aralkyl (e.g. benzyl), aryloxyalkyl (e.g. phenoxymethyl), aryl (e.g. phenyl optionally substituted by halogen, —C₁₋₄alkyl or —C₁₋₄alkoxy or amino); or for example —CH₂OC(O)R′ or —CH₂OCO₂R′ in which R′ is alkyl (e.g. R′ is t-butyl). Unless otherwise specified, any alkyl moiety present in such esters preferably contains 1 to 18 carbon atoms, particularly 1 to 4 carbon atoms. Any aryl moiety present in such esters preferably comprises a phenyl group.

As used herein, the term “pharmaceutically acceptable” used in relation to an ingredient (active ingredient such as an active ingredient, a salt thereof or an excipient) which may be included in a pharmaceutical formulation for administration to a patient, refers to that ingredient being acceptable in the sense of being compatible with any other ingredients present in the pharmaceutical formulation and not being deleterious to the recipient thereof.

Throughout the specification and the claims which follow, unless the context requires otherwise, the word ‘comprise’, and variations such as ‘comprises’ and ‘comprising’, will be understood to imply the inclusion of a stated integer or step or group of integers but not to the exclusion of any other integer or step or group of integers or steps.

It will further be appreciated that certain compounds of the present invention may exist in different tautomeric forms. All tautomers are contemplated to be within the scope of the present invention.

### Processes

Compounds of Formula (I) in which A is hydroxy may be prepared from a compound of Formula (II)

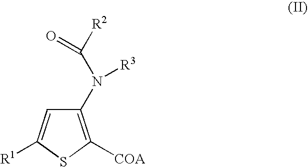

in which A is a protected hydroxy group, for example an alkoxy, benzyloxy or silyloxy group and R¹, R², and R³ are as defined above for Formula (I). For example when A is methoxy or ethoxy, and R¹, R² and R³ are as defined above for Formula (I), by treatment with an appropriate base, for example aqueous sodium hydroxide or lithium hydroxide, optionally in a suitable solvent such as methanol, tetrahydrofuran or combinations thereof. Suitably, the temperature is in the range 25 to 100° C., more preferably 50 to 100° C. Alternatively, when A is methoxy or ethoxy and R¹, R² and R³ are as defined above for Formula (I), by treatment with lithium iodide in a suitable solvent such as pyridine, lutidine or collidine, suitably in the temperature range 100-170° C.

For example when A is tert-butoxy, and R¹, R² and R³ are as defined above for Formula (I), by treatment with an appropriate acid, for example trifluoroacetic acid. Suitably, the reaction is carried out in a solvent, for example dichloromethane. Suitably, the temperature is in the range 0 to 50° C., more preferably 15 to 30° C.

For example when A is silyloxy, and R¹, R² and R³ are as defined above for Formula (I), by treatment with a suitable fluoride source for example tetrabutylammonium fluoride. The reaction is carried out in a suitable solvent, for example tetrahydrofuran. Suitably, the temperature is in the range 0 to 50° C., more preferably 15 to 30° C.

Compounds of Formula (I) in which A is hydroxy, or (II) in which A is an alkoxy, benzyloxy or silyloxy group and R¹ and R³ are as defined above for Formula (I), may be prepared by reaction of a compound of Formula (III)

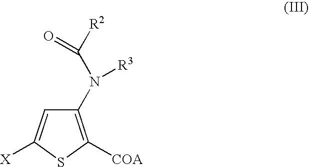

in which A is hydroxy or an alkoxy, benzyloxy or silyloxy group, and R² and R³ are as defined above for Formula (I) and X is a halo atom such as bromo or iodo; with a suitable boronic acid R¹—B(OH)₂ or boronate ester R¹—B(OR′)(OR″), in which R′ and R″ are independently alkyl or R′ and R″ together with the carbon atoms to which they are attached form a ring optionally substituted by alkyl, such as a pinacol ester, in the presence of a palladium catalyst such as tetrakis(triphenylphosphine) palladium or bis-[(diphenylphosphino)-ferrocene]palladium(II) chloride, in the presence of a suitable base such as sodium carbonate, in a suitable solvent such as DMF, methanol or toluene, or combinations thereof, at a temperature in the range 50-10° C., preferably under an inert atmosphere.

Compounds of Formula (III) in which A is an alkoxy, benzyloxy or silyloxy group may be prepared from compounds of Formula (IV)

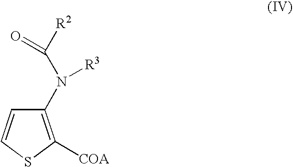

in which A is an alkoxy, benzyloxy or silyloxy, and R² and R³ are as defined above for Formula (I), by treatment with a suitable base such as lithium diisopropylamide and a halogen source such as bromine, iodine, N-bromosuccinide or N-iodosuccinimide in a suitable solvent such as tetrahydrofuran, and at a temperature in the range −78 to −20° C.

Compounds of Formula (III) in which A is hydroxy may be prepared from compounds of Formula (III) in which A is an alkoxy, benzyloxy or silyloxy group, for example by treatment with an appropriate base, acid or fluoride source as described in relation to the preparation of compounds of Formula (I) from compounds of Formula (II).

Compounds of Formula (IV) may be prepared by reaction of a compound of Formula (V)

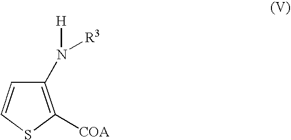

in which A is an alkoxy, benzyloxy or silyloxy group, and R³ is as defined above for Formula (I); with a suitable acylating agent, for example R²—C(O)—Y, wherein Y is a halo atom, for example chloro or bromo, and R² is as defined above for Formula (I). The reaction may be carried out in a suitable solvent, for example dichloromethane or dichloroethane, optionally in the presence of a suitable base, for example pyridine or triethylamine. A phosphine such as triphenylphosphine may optionally be used in place of the base.

Compounds of Formula (V) may be prepared by reaction of a compound of Formula (VI)

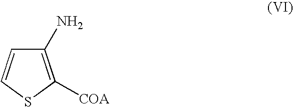

in which A an alkoxy, benzyloxy or silyloxy group, by treatment with a suitable vinyl ether, or a suitable aldehyde or a suitable ketone, in the presence of a suitable acid, such as acetic acid, and a suitable reducing agent such as sodium triacetoxyborohydride, in a suitable solvent such as dichloromethane. Alternatively, compounds of Formula (V) may be prepared from compounds of Formula (VI) in which A is an alkoxy, benzyloxy or silyloxy group, by treatment with a suitable alkylating agent R³-X′ where X′ is a halo atom such as chloro, bromo or iodo, or X′ is a sulphonate ester such as methanesulfonate, in a suitable solvent, such as dimethylformamide, in the presence of a suitable base, such as triethylamine.

Compounds of Formula (V) may also be prepared by reacting a compound of Formula (VII)

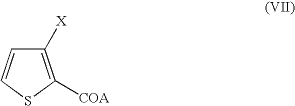

in which A is an alkoxy, benzyloxy or silyloxy group and X is a halo atom such as bromo, with an amine R³NH₂ in the presence of a palladium catalyst such as tris(dibenzylidenacetone)dipalladium, in the presence of a reagent, such as BINAP, and a base, such as cesium carbonate, in a suitable solvent, such as toluene, and at a temperature in a suitable range, such as 80-120° C.

Compounds of Formula (IV) may also be prepared by reaction of a compound of Formula (VIII)

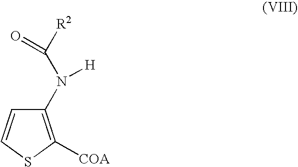

in which A is an alkoxy, benzyloxy or silyloxy group, and R² is as defined above for Formula (I); with a suitable alkylating agent R³—X′ in which X′ is a halo atom such as chloro, bromo or iodo, or X′ is a sulphonate ester such as methanesulfonate, in a suitable solvent such as dimethylformamide, in the presence of a suitable base, such as triethylamine or sodium hydride.

Compounds of Formula (VIII) may be prepared by reaction of a compound of Formula (VI) in which A is an alkoxy, benzyloxy or silyloxy group, with a suitable acylating agent, for example R²—C(O)—Y, wherein Y is a halo atom, such as chloro or bromo, and R² is as defined above for Formula (I). The reaction may be carried out in a suitable solvent, for example dichloromethane, in the presence of a suitable base, for example pyridine or triethylamine. A phosphine such as triphenylphosphine may optionally be used in place of the base.

Compounds of Formula (II) may also be prepared by reaction of a compound of Formula (IX)

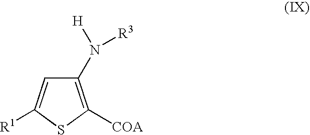

in which A is an alkoxy, benzyloxy or silyloxy group, and R¹ and R³ are as defined above for Formula (I), with a suitable acylating agent, for example R²—C(O)—Y, wherein Y is a halo atom, such as chloro or bromo, and R² is as defined above for Formula (I). The reaction may be carried out in a suitable solvent, for example dichloromethane, in the presence of a suitable base, for example pyridine or triethylamine. A phosphine such as triphenylphosphine may optionally be used in place of the base.

Compounds of Formula (IX) may be prepared by reaction of a compound of Formula (X)

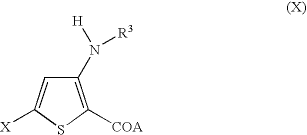

in which X is a halo atom such as bromo or iodo, with a suitable boronic acid R¹—B(OH)₂ or boronate ester R¹—B(OR′)(OR″), in which R′ and R″ are independently alkyl or R′ and R″ together with the carbon atoms to which they are attached form a ring optionally substituted by alkyl, such as a pinacol ester, in the presence of a palladium catalyst such as tetrakis(triphenylphosphine) palladium or bis-[(diphenylphosphino)-ferrocene]palladium(II) chloride, in the presence of a suitable base such as sodium carbonate, in a suitable solvent such as DMF, methanol or toluene, or combinations thereof, at a temperature in the range 50-100° C., preferably under an inert atmosphere.

Compounds of Formula (X) may be prepared by reaction of a compound of Formula (XI)

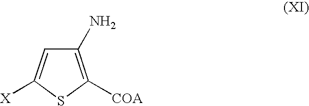

in which A is an alkoxy, benzyloxy or silyloxy group, and X is a halo atom, such as bromo or iodo, by treatment with a suitable vinyl ether, or a suitable aldehyde or a suitable ketone, in the presence of a suitable acid, such as acetic acid, and a suitable reducing agent, such as sodium triacetoxyborohydride, in a suitable solvent, such as dichloromethane. Alternatively, compounds of Formula (X) may be prepared from compounds of Formula (XI) in which A is an alkoxy, benzyloxy or silyloxy, and X is a halo atom, such as bromo or iodo, by treatment with a suitable alkylating agent R³-X′ where X′ is a halo atom such as chloro, bromo or iodo, or X′ is a sulphonate ester such as methanesulfonate, in suitable solvent, such as dimethylformamide, in the presence of a suitable base, such as triethylamine.

Compounds of Formula (XI) may be prepared by hydrolysis of a compound of Formula (XII)

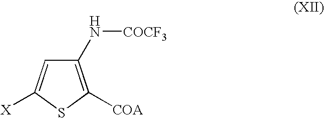

in which A is an alkoxy, benzyloxy or silyloxy group and X is a halo atom, such as bromo or iodo, with a suitable base, such as aqueous potassium carbonate, optionally in the presence of an alcohol, such as methanol.

Compounds of Formula (XII) may be prepared by reaction of a compound of Formula (XIII)

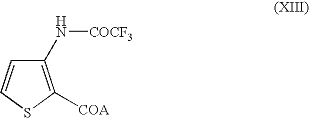

in which A is an alkoxy, benzyloxy or silyloxy group, with a suitable base, such as lithium diisopropylamide and a halogen source, such as bromine, iodine, N-bromosuccinide, or N-iodosuccinimide, in a suitable solvent, such as tetrahydrofuran, at a temperature in the range −78 to −20° C.

Compounds of Formula (XIII) may be prepared by treating compounds of Formula (VI) with trifluoroacetic anhydride in a suitable solvent, such as ether.

Compounds of Formula (III) in which A is an alkoxy, benzyloxy or silyloxy group may also be prepared from compounds of Formula (XIV)

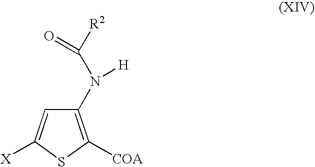

in which A is an alkoxy, benzyloxy or silyloxy group, X is a halo atom, such as bromo or iodo, and R² is as defined above for Formula (I), by treatment with a suitable alkylating agent R³-X′ where X′ is a halo atom such as chloro, bromo or iodo, or X′ is a sulphonate ester such as methanesulfonate, in a suitable solvent, such as dimethylformamide, in the presence of a suitable base, such as triethylamine or sodium hydride.

Compounds of Formula (XIV) may be prepared from compounds of Formula (XI) by reaction with a suitable acylating agent, for example R²—C(O)—Y, wherein Y is a halo atom, such as chloro or bromo, and R² is as defined above for Formula (I). The reaction may be carried out in a suitable solvent, for example dichloromethane, in the presence of a suitable base, for example pyridine or triethylamine. A phosphine such as triphenylphosphine may optionally be used in place of the base.

Compounds of Formula (VI) and (VII) are commercially available or well known in the art.

Compounds of Formula (I) in which A is hydroxy, or (II) in which A is an alkoxy, benzyloxy or silyloxy group, may be prepared by reaction of a compound of Formula (II)′

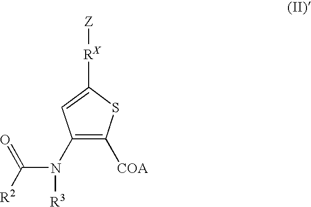

in which Z represents a halo atom, such as chloro, bromo or iodo, and R^{X}, R², R³ are as defined above for Formula (I), and A is hydroxy or an alkoxy, benzyloxy or silyloxy group, by reaction with a suitable heteroaryl boronic acid, R^{Y}-boronic acid, in which R^{Y} is as defined above for Formula (I), in the presence of a palladium catalyst such as palladium (II) acetate, a reagent such as 2-dicyclohexylphosphino-2′(N,N-dimethylamino)-biphenyl, and an additional reagent such as caesium fluoride, in a suitable solvent, such as dioxane. Alternatively the R^{Y} boronic acid or boronic ester may be reacted in the presence of a palladium catalyst such as tetrakis(triphenylphosphonium)palladium, a reagent such as sodium carbonate, is a suitable solvent such as dimethoxymethane or ethanol, preferably at a temperature in the range 50-85° C. Alternatively, for coupling the boronic acids or esters methods well known in the art may be employed, see, for example, Chemical Communications (2005) 38, 4759-4763, Angew Chemie Int Ed (2005) 44, 4442-4489, Tetrahedron (2002) 58, 9633-9695, Synthesis (2004) 2419-2440.

Compounds of Formula (I) in which A is hydroxy, or (II) in which A is an alkoxy, benzyloxy or silyloxy group, may also be prepared by reaction of a compound of Formula (II)′

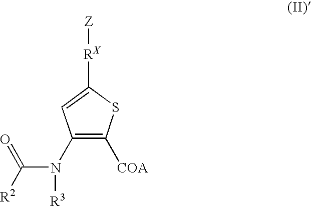

in which Z represents B(OH)₂, and R^{X}, R², R³ are as defined above for Formula (I), and A is hydroxy or an alkoxy, benzyloxy or silyloxy group, by reaction with a suitable heteroaryl halide, R^{Y}-hal, in which R^{Y} is as defined above for Formula (I) and suitably hal is bromo or iodo, in the presence of a palladium catalyst such as palladium (II) acetate, a reagent such as 2-dicyclohexylphosphino-2′(N,N-dimethylamino)-biphenyl, and an additional reagent such as caesium fluoride, in a suitable solvent such as dioxane. Alternatively the R^{Y} boronic acid or boronic ester may be reacted in the presence of a palladium catalyst such as tetrakis(triphenylphosphonium)palladium, a reagent such as sodium carbonate, is a suitable solvent such as dimethoxymethane or ethanol, preferably at a temperature in the range 50-85° C. Alternatively, for coupling the boronic acids or esters methods well known in the art may be employed, see, for example, Chemical Communications (2005) 38, 4759-4763, Angew Chemie Int Ed (2005) 44, 4442-4489, Tetrahedron (2002) 58, 9633-9695, Synthesis (2004) 2419-2440.

Compounds of Formula (II)′ in which Z is halo may be prepared by reaction of a compound of Formula (III) with a boronic acid of Formula Z-R^{X}-boronic acid under the conditions described above for the preparation of compounds of Formula (I) and (II) from (III) and R¹-boronic acid.

Compounds of Formula (II)′ in which Z is B(OH)₂ may be prepared by reaction of a compound of Formula (III) with a compound of Formula Z-R^{X}—B(OH)₂ under the conditions described above for the preparation of compounds of Formula (I) and (II) from (III) and R¹-boronic acid.

Boronic acids Z-R^{X}-boronic acid, R¹-boronic acid and R^{Y}-boronic acid are commercially available or may be prepared by analogy to methods provided in Organometallics (1983) 2, 1316, Chem. Revs. (1995) 95, 2457, Journal of Org Chem (2004) 69, 1999, SynLett (2004) (5), 892, Bioorg Med Chem (2005) 13, 2305, Tetrahedron Letters (2004) 44, 9359 and Tetrahedron Letters (2005) 45, 6657.

Compounds of Formula (I) or (II) in which R¹ represents a 4-(furopyridine)phenyl, may be prepared by treatment of a compound of Formula (II)″

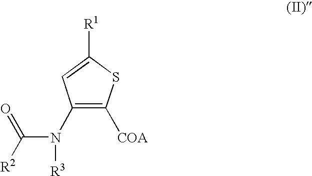

in which R¹ represents a 4-ethynylphenyl derivative, and R², R³, R⁴ and A are as defined above for Formula (II), with a suitable pyridine (the pyridine being substituted with adjacent hydroxy and iodo groups), with a suitable catalyst such as bis(triphenylphosphine)palladium (II) chloride and copper (I) iodide, in the presence of a suitable base, such as triethylamine, optionally in an additional suitable solvent, such as DMF. Suitably the temperature is in the range 50-80° C. For examples of furopyridine synthesis see Bioorganic and Medicinal Chemistry Letters (2002) 12, 1399, Synthesis (1986) 749.

Compounds of Formula (I) or (II) in which R¹ represents a 4-(pyrrolopyridine)phenyl, may be prepared by treatment of a compound of Formula (II)″ in which R¹ represents 4-ethynylphenyl with an appropriate pyridine (the pyridine being substituted by adjacent amino and iodo groups), in the presence of a suitable catalyst such as bis(triphenylphosphine)palladium (II) chloride and copper (I) iodide, in a suitable solvent such as triethylamine. Suitably the temperature is in the range 50-80° C. For examples of pyrrolopyridine synthesis see Heterocycles (1986) 24, 31, Tetrahedron (2003) 59, 1571, Synlett (1992) 515.

Compounds of Formula (I) or (II) in which R¹ represents phenyl substituted by a imidazo[1,2-a]pyridine, may be prepared by analogy to methods described in Tetrahedron Letters (2001) 42, 3077.

Compounds of Formula (I) or (II) in which R¹ represents a 4-(1H-benzimidazol-2-yl)phenyl derivative may be prepared by analogy to methods described in J. Heterocyclic Chem. (1994)31, 957.

Compounds of Formula (I) or (II) in which R¹ represents a 4-(1,3-benzoxazol-2-yl)phenyl derivative may be prepared by analogy to methods described in Tetrahedron Letters (2003) 44, 175.

Compounds of Formula (I) or (II) in which R¹ represents a 4-(1,3-benzothiazol-2-yl)phenyl derivative may be prepared by analogy to methods described in Tetrahedron Letters (2003) 44, 175 or Synth. Commun. (1990) 20, 3379.

Compounds of Formula (I) or (II) in which R¹ represents a 4-(oxazolopyridine)phenyl may be prepared by reacting a compound of Formula (II)″ in which R¹ represents 4-carboxyphenyl with an appropriate pyridine derivative (the pyridine being substituted with adjacent amino and hydroxyl groups), in the presence of an acid such as polyphosphoric acid at temperatures in the range 180-200° C. (see for example J. Med. Chem. (1978) 21, 1158). Alternatively, the acid chloride of the 4-carboxyphenyl may be reacted with an appropriate pyridine (the pyridine being substituted with adjacent amino and hydroxyl groups) in a microwave reactor in a suitable solvent such as dioxan (see for example Tetrahedron Letters (2003) 44, 175). Compounds of Formula (II) in which R¹ represents a 4-(oxazolopyridine)phenyl may also be prepared by reacting the 4-carboxyphenyl derivative with an appropriate pyridine (the pyridine being substituted with adjacent amino and hydroxyl groups) using a suitable coupling agent such as HATU, and then in a second step cyclised using an appropriate reagent such as phosphorous oxychloride.

Compounds of Formula (I) or (II) in which R¹ represents a 4-(thiazolopyridine)phenyl, may be prepared by reacting a compound of Formula (II)″ in which R¹ represents 4-phenyl-COCl with an appropriate pyridine (the pyridine being substituted with adjacent amino and chloro groups), in the presence of a suitable base such as pyridine, and then in a second step cyclised using a reagent such as Lawesson's reagent in a suitable solvent such as DMPU, at a suitable temperature such as 90-110° C.

Compounds of Formula (I) or (II) in which R¹ represents a 4-(thiazolopyridine)phenyl, may also be prepared by reacting a compound of Formula (II)″ in which R¹ represents 4-carboxyphenyl with an appropriate pyridine (the pyridine being substituted with adjacent amino and thiol groups), in the presence of an acid such as polyphosphoric acid at temperatures in the range 180-200° C. (see for example J. Med. Chem. (1978) 21, 1158). Alternatively, the acid chloride of the 4-carboxyphenyl may be reacted with an appropriate pyridine (the pyridine being substituted with adjacent amino and thiol groups), in a microwave reactor in a suitable solvent such as dioxan (see for example Tetrahedron Letters (2003) 44, 175). In another alternative the 4-carboxyphenyl compound may be reacted with an appropriate pyridine (the pyridine being substituted with adjacent amino and thiol groups), using a suitable coupling agent such as HATU, and then in a second step cyclised using an appropriate reagent such as phosphorous oxychloride.

Compounds of Formula (I) or (II) in which R¹ represents a 4-(2,3-dihydro-1,1-dioxo-1,2-benzisothiazol-2(3H)-yl)phenyl derivative, may be prepared by treatment of a compound of Formula (II)′ in which Z represents 4-halo with a 2,3-dihydro-1,2-benzisothiazole 1,1-dioxide derivative in the presence of copper (I) iodide with a suitable base such as potassium carbonate, and in the presence of a reagent such as trans-1,2-diaminocyclohexane or trans-N,N′-dimethyl-1,2-cyclohexanediamine, or a combination thereof in a suitable solvent such as dioxan, DMF or pyridine or a combination thereof, and at a temperature in the range 90-160° C.

Compounds of Formula (I) or (II) in which R¹ represents a 4-(1-oxo-1,3-dihydro-2H-isoindol-2-yl)phenyl derivative, may be prepared by treatment of a compound of Formula (II)″ in which R¹ represents a 4-aminophenyl derivative with a phenyl-1,2-di-aldehyde derivative in acetic acid optionally with a suitable solvent such as dichloromethane. Alternatively, compounds of Formula (I) or (II) in which in which R¹ represents a 4-(1-oxo-1,3-dihydro-2H-isoindol-2-yl)phenyl derivative may also be prepared by treatment of a compound of Formula (II)″ in which R¹ represents 4-aminophenyl with a suitable phenyl derivative (this being substituted with adjacent methyl ester and bromomethyl groups), in the presence of a suitable base such as diisopropylethylamine, in a suitable solvent such as acetonitrile.

Compounds of Formula (I) or (II) in which R¹ represents a 4-((1-oxo-1,3-dihydro-2H-azaisoindol-2-yl)phenyl derivative, may be prepared by treatment of a compound of Formula (II)″ in which R¹ represents 4-aminophenyl with an appropriate pyridine derivative (the pyridine being substituted with adjacent methyl ester and bromomethyl groups), in the presence of a suitable base such as diisopropylethylamine in a suitable solvent such as acetonitrile.

Compounds of Formula (I) or (II) in which R¹ represents a 4-(pyrazolopyrimidine)phenyl, may be prepared by treating a compound of Formula (II)″ in which R¹ represents 4-(phenyl)-1H-pyrazole-5-amine with 1,1,3,3-tetramethoxypropane in a suitable solvent, such as acetic acid, suitably the temperature is in the range 90-110° C.

Suitable methods for the preparation of compounds with the above discussed R^{Y} derivatives may be found in the chemical literature, for example those described in Comprehensive Heterocyclic Chemistry, Edited by A. R. Katritzky and C. W. Rees, Pergamon 1984, and Heterocyclic Chemistry, Edited by J. A. Joules and K. Mills, 4^{th} Ed, Blackwell Science.

Compounds of Formula R^{Y}—R^{X}—B(OR′)₂ or R^{Y}—B(OR′)₂ for use in the preparation of compounds of Formula (II) are available commercially or may be prepared from compounds of Formula R^{Y}-R^{X}-hal or R^{Y}-hal by methods well known in the art.

Compounds of Formula R^{Y}-R^{X}-hal or R^{Y}-hal for use in the preparation of compounds of Formula (II) are available commercially or may be prepared by methods well known in the art. Representative examples of heteroaryl halide preparation are given below (but are not exhaustive).

A 2-(4-bromophenyl)-2H indazole derivative may be prepared by analogy to methods described in Farmaco Ed Sci (1981) 36, 1037 or J. Chem. Soc. Perkin Trans 2 (1975), 1185, for example by treating 2-nitrobenzaldehyde with 4-bromoaniline in a suitable solvent such as methanol, and in a separate step reacting the imine with a phosphite such as triethylphosphite, in a microwave reactor at 210° C. for 20 mins.

A 2-(4-bromophenyl)imidazo[1,2-a]pyridine derivative may be prepared by analogy to methods described in Tetrahedron Letters (2001) 42, 3077.

A 2-(4-bromophenyl)-1H-benzimidazole derivative may be prepared by analogy to methods described in J. Heterocyclic Chem. (1994) 31, 957.

A 2-(4-bromophenyl)-1H-benzoxazole derivative may be prepared by analogy to methods described in Tetrahedron Letters (2003) 44, 175.

A 2-(4-bromophenyl)-1H-benzothiazole derivatives may be prepared by analogy to methods described in Tetrahedron Letters (2003) 44, 175 or Synth. Commun. (1990) 20, 3379.

A 4-(furopyridine)phenyl bromide, may be prepared by treatment of a 4-ethynylphenyl bromide with a suitable pyridine (the pyridine being substituted with adjacent hydroxy and iodo groups), with a suitable catalyst such as bis(triphenylphosphine)palladium (II) chloride and copper (I) iodide, in a suitable solvent such as triethylamine or DMF. Suitably the temperature is in the range 50-80° C. For examples of furopyridine synthesis see Bioorganic and Medicinal Chemistry Letters (2002) 12, 1399, Synthesis (1986) 749.

A 4-(oxazolopyridine)phenyl bromide may be prepared by reacting a 4-carboxyphenyl bromide with an appropriate pyridine derivative (the pyridine being substituted with adjacent amino and hydroxyl groups), in the presence of an acid such as polyphosphoric acid at temperatures in the range 180-200° C. (see for example J. Med. Chem. (1978) 21, 1158). Alternatively, the acid chloride of the 4-carboxyphenyl bromide may be reacted with an appropriate pyridine (the pyridine being substituted with adjacent amino and hydroxyl groups) in a microwave reactor in a suitable solvent such as dioxane (see for example Tetrahedron Letters (2003) 44, 175). A 4-(oxazolopyridine)phenyl bromide may also be prepared by reacting a 4-carboxyphenyl bromide derivative with an appropriate pyridine (the pyridine being substituted with adjacent amino and hydroxyl groups) using a suitable coupling agent such as HATU, and then in a second step cyclised using an appropriate reagent such as phosphorous oxychloride.

A 4-(thiazolopyridine)phenyl bromide may be prepared by reacting an appropriate 4-carboxyphenyl bromide with an appropriate pyridine (the pyridine being substituted with adjacent amino and thiol groups), in the presence of an acid such as polyphosphoric acid at temperatures in the range 180-200° C. (see for example J. Med. Chem. (1978) 21, 1158). Alternatively, the acid chloride of the 4-carboxyphenyl may be reacted with an appropriate pyridine (the pyridine being substituted with adjacent amino and thiol groups), in a microwave reactor in a suitable solvent such as dioxan (see for example Tetrahedron Letters (2003) 44, 175). In another alternative the 4-carboxyphenyl bromide may be reacted with an appropriate pyridine (the pyridine being substituted with adjacent amino and thiol groups), using a suitable coupling agent such as HATU, and then in a second step cyclised using an appropriate reagent such as phosphorous oxychloride.

A 4-(thiazolopyridine)-phenyl bromide may be prepared by reacting an appropriate 4-bromophenyl-COCl with an appropriate pyridine (the pyridine being substituted with adjacent amino and chloro groups), in the presence of a suitable base such as pyridine, and then in a second step is cyclised using a reagent such as Lawesson's reagent in a suitable solvent such as DMPU, at a suitable temperature such as 90-110° C.

A 4-(1-oxo-1,3-dihydro-2H-isoindol-2-yl)phenyl bromide derivative may be prepared by treatment of an appropriate 4-aminophenyl bromide derivative with a phenyl-1,2-di-aldehyde derivative in acetic acid optionally with a suitable solvent such as dichloromethane. A 4-(1-oxo-1,3-dihydro-2H-isoindol-2-yl)phenyl bromide derivative may also be prepared by treatment of an appropriate 4-aminophenyl bromide with a suitable phenyl derivative (this being substituted with adjacent methyl ester and bromomethyl groups), in the presence of a suitable base such as diisopropylethylamine, in a suitable solvent such as acetonitrile.

A 4-(1-oxo-1,3-dihydro-2H-azaisoindol-2-yl)phenyl bromide may be prepared by treatment of an appropriate 4-aminophenyl bromide with an appropriate pyridine derivative (the pyridine being substituted with adjacent methyl ester and bromomethyl groups), in the presence of a suitable base such as diisopropylethylamine in a suitable solvent such as acetonitrile.

A 4-(pyrazolopyrimidine)phenyl bromide may be prepared by treating a 3-(4-bromophenyl)-1H-pyrazole-5-amine with 1,1,3,3-tetramethoxypropane in a suitable solvent such as acetic acid, suitably the temperature is in the range 90-110° C.

A 4-(pyrazolopyrimidine)thienyl bromide may be prepared by treating a bromothienyl-1H-pyrazole-5-amine with 1,1,3,3-tetramethoxypropane in a suitable solvent such as acetic acid, suitably the temperature is in the range 90-110° C.

Esters of compounds of Formula (I), in which A is —OR where R is selected from straight or branched chain alkyl, aralkyl, aryloxyalkyl, or aryl, may also be prepared by esterification of a compound of Formula (I) in which A is hydroxy by standard literature procedures for esterification.

The various general methods described above may be useful for the introduction of the desired groups at any stage in the stepwise formation of the required compound, and it will be appreciated that these general methods can be combined in different ways in such multi-stage processes. The sequence of the reactions in multi-stage processes should of course be chosen so that the reaction conditions used do not affect groups in the molecule which are desired in the final product.

It will be appreciated that compounds of Formula (I), (II), (II)′, (II)″, (III), (IV), (V), (VIII), (IX), (X) and (XIV) which exist as diastereoisomers may optionally be separated by techniques well known in the art, for example by column chromatography or recrystallisation. For example, the formation of an ester using a chiral alcohol, separation of the resulting diastereoisomers, and subsequent hydrolysis of the ester to yield the individual enantiomeric acid of Formula (I), (II), (II)′, (II)″, (III), (IV), (V), (VIII), (IX), (X) and (XIV).

It will be appreciated that racemic compounds of Formula (I), (II), (II)′, (II)″, (III), (IV), (V), (VIII), (IX), (X) and (XIV) may be optionally resolved into their individual enantiomers. Such resolutions may conveniently be accomplished by standard methods known in the art. For example, a racemic compound of Formula (I), (II), (II)′, (II)″, (III), (IV), (V), (VIII), (IX), (X) and (XIV) may be resolved by chiral preparative HPLC. Alternatively, racemic compounds of Formula (I), (II), (II)′, (II)″, (III), (IV), (V), (VIII), (IX), (X) and (XIV) which contain an appropriate acidic or basic group, such as a carboxylic acid group or amine group may be resolved by standard diastereoisomeric salt formation with a chiral base or acid reagent respectively as appropriate. Such techniques are well established in the art. For example, a racemic basic compound may be resolved by treatment with a chiral acid such as (R)-(−)-1,1′-binaphthyl-2,2′-diyl-hydrogen phosphate or (−)-di-O,O′-p-tolyl-L-tartaric acid, in a suitable solvent, for example isopropanol. The free enantiomer may then be obtained by treating the salt with a suitable base, for example triethylamine, in a suitable solvent, for example methyl tert-butyl ether. Alternatively, racemic acid compounds may be resolved using a chiral base, for example (S)-alpha methylbenzylamine, (S)-alpha phenylethylamine, (1S,2S)-(+)-2-amino-1-phenyl-1,3-propane-diol, (−) ephidrine, quinine, brucine. Individual enantiomers of Formula (II), (II)′, (II)″, (III), (IV), (V), (VIII), (IX), (X) and (XIV) may then be progressed to an enantiomeric compound of Formula (I) by the chemistry described above in respect of racemic compounds.

With appropriate manipulation and protection of any chemical functionality, synthesis of compounds of Formula (I) is accomplished by methods analogous to those above and to those described in the Experimental section. Suitable protecting groups can be found, but are not restricted to, those found in T W Greene and P G M Wuts ‘Protective Groups in Organic Synthesis’, 3^{rd} Ed (1999), J Wiley and Sons.

Various of the synthetic procedures described above in general terms (and below in specific terms) may involve heating the reactants. It will be appreciated that heating may be carried out by various conventional methods but also with use of a microwave oven.

### EXAMPLES

### Abbreviations

AcOH acetic acid
DBU 1,8-diazabicyclo[5.4.0]undec-7-ene
DCE 1,2-dichloroethane
DCM dichloromethane
DEF N,N-diethylformamide
DMAP 4-dimethylaminopyridine
DME 1,2-dimethoxyethane
DMF N,N-dimethylformamide
EtOAc ethyl acetate
Et₂O diethyl ether
h hours
HCl hydrochloric acid
HPLC high pressure liquid chromatography
ISCO Companion automated flash chromatography equipment with fraction analysis by UV absorption available from Presearch.
IPA isopropyl alcohol
LDA lithium diisopropylamide
MDAP HPLC reverse phase HPLC on a C₁₈ column using a two-solvent gradient elution with (A) water containing formic acid (0.1%) and (B) acetonitrile-water (95:5 v/v) containing formic acid (0.05%) as the eluents, and analysis of the fractions by electrospray mass spectroscopy.
MeCN acetonitrile
MeOH methanol
MIBK methyl isobutyl ketone
mins minutes
NH₂ SPE aminopropyl ion exchange cartridge
OASIS HLB cartridge sample extraction cartridge available from Waters
SCX-2 propylsulphonic acid ion exchange cartridge SPE solid phase extraction column TFA trifluoroacetic acid
THF tetrahydrofuran

All mass spectroscopy was performed using electrospray as the method of ionisation.

### Intermediate 1

### Methyl 3-[(trifluoroacetyl)amino]-2-thiophenecarboxylate

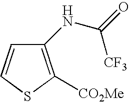

To a solution of methyl 3-amino-2-thiophenecarboxylate (5 g) in ether (100 mL) at 0° C. under nitrogen was added slowly trifluoroacetic anhydride (4.5 mL). The reaction was stirred at room temperature for 2 h. The reaction was partitioned between diethyl ether and 2N hydrochloric acid. The organics were washed with brine, dried (Na₂SO₄) and evaporated to give the title compound.

MS calcd for (C₈H₆F₃NO₃S—H)⁻: 252

MS found (electrospray): (M−H)⁻=252

### Intermediate 2

### Methyl 5-iodo-3-[(trifluoroacetyl)amino]-2-thiophenecarboxylate

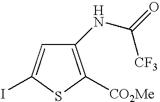

To a solution of lithium diisopropylamide (26 mL, 2M solution in heptane/tetrahydrofuran) in THF (80 mL) at −78° C. under nitrogen was added drop wise a solution of Intermediate 1 (4 g) in THF (60 mL). The reaction was stirred for 10 min and iodine (12 g) in THF (60 mL) was added slowly to the reaction. Stirring was continued at −78° C. under nitrogen for a further 1.5 h. The reaction was neutralised with ammonium chloride and washed with 5% sodium thiosulphate solution. The organics were washed with brine, dried (Na₂SO₄) and concentrated to give an oil. The crude product was then purified by Biotage silica chromatography eluting with EtOAc in cyclohexane (2.5%) to give the title compound.

MS calcd for (C₈H₅F₃INO₃S—H)⁻: 378

MS found (electrospray): (M−H)⁻=378

### Intermediate 3

### Methyl 3-amino-5-iodo-2-thiophenecarboxylate

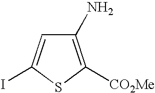

Potassium carbonate (8.1 g) was added in one portion to Intermediate 2 (3.7 g) in MeOH (130 mL) and water (18.5 mL) at room temperature. The reaction was stirred for 1 h, then partitioned between water and EtOAc. The organics were washed with brine, dried (Na₂SO₄) and concentrated to give the title compound.

MS calcd for (C₆H₆₁NO₂S+H)⁺: 284

MS found (electrospray): (M+H)⁺=284

### Intermediate 4

### Methyl 5-iodo-3-[(1-methylethyl)amino]-2-thiophenecarboxylate

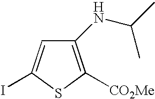

To Intermediate 3 (4.4 g) in dry DCM (80 mL) was added 2-methoxypropene (6 mL) and acetic acid slowly (3.6 mL), followed by sodium triacetoxyborohydride (6.6 g) added portion wise while controlling the temperature. The mixture was stirred at room temperature under nitrogen overnight. The reaction was neutralised by slowly adding sodium bicarbonate and extracted with DCM. The organics were dried (Na₂SO₄) and concentrated. The crude product was then purified by Biotage silica chromatography eluting with EtOAc in cyclohexane (2.5%) to give the title compound.

MS calcd for (C₉H₁₂INO₂S+H)⁺: 326

MS found (electrospray): (M+H)⁺=326

### Alternative Preparation of Intermediate 4 (Method B)

### Methyl 5-iodo-3-[(1-methylethyl)amino]-2-thiophenecarboxylate

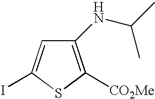

A solution of LDA (2.0M solution in THF/heptane/ethyl benzene, 15.2 mL) was cooled to an internal temperature of −78° C. under nitrogen. Intermediate 54 (3 g) was dissolved in dry THF (30 mL) and this was added slowly to the cooled LDA solution, maintaining an internal temperature between −73° C. and −65° C. After stirring at −74° C. for 1 h a solution of iodine (5.1 g) in dry THF (30 mL) was added dropwise over 30 mins maintaining an internal temperature less than −65° C. After 25 mins the reaction was quenched with saturated NH₄Cl solution (15 mL) and was allowed to warm to room temperature, before being washed with 5% sodium thiosulphate solution. The organics were separated and the aqueous layer was extracted with EtOAc (×2). The combined organics were dried with sodium sulphate, filtered and concentrated in vacuo. The crude material was purified by ISCO Companion silica chromatography, eluting with a gradient of 0-50% EtOAc in cyclohexane to give the title compound.

MS calcd for (C₉H₁₂INO₂S+H)⁺: 326

MS found (electrospray): (M+H)⁺=326

### Intermediate 5

### Methyl 5-iodo-3-[[(trans-4-methylcyclohexyl)carbonyl](1-methylethyl)amino]-2-thiophenecarboxylate

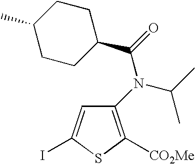

To methyl 5-iodo-3-[(1-methylethyl)amino]-2-thiophenecarboxylate (2.85 g, a preparation of which is described above as Intermediate 4) in dry DCM (8 mL) was added trans-4-methylcyclohexanecarbonyl chloride¹ (1.8 mL) slowly and then triphenylphosphine (3.9 g) in dry DCM (7 mL) dropwise. The reaction was heated at 40° C. under nitrogen for 24 h. More acid chloride (0.5 mL) was added and the reaction heated at 40° C. for 24 h. A further quantity of acid chloride (1.3 mL) and triphenylphosphine (3.5 g) were added to the reaction and left to stir at room temperature for 2 days. The reaction was then partitioned between DCM and saturated sodium hydrogen carbonate solution, dried (Na₂SO₄) and concentrated. This was purified by Biotage silica chromatography eluting with DCM/cyclohexane (1:4) then EtOAc/cyclohexane (1:9). This was further purified by ISCO companion silica chromatography, eluting with a gradient of ethyl acetate in cyclohexane (0-20%) to give the title compound.

MS calcd for (C₁₇H₂₄INO₃S+H)⁺: 450

MS found (electrospray): (M+H)⁺=450

Ref 1: WO 2004/052885.

### Alternative preparation of Intermediate 5 (Method B)

### Methyl 5-iodo-3-[[(trans-4-methylcyclohexyl)carbonyl](1-methylethyl)amino]-2-thiophenecarboxylate

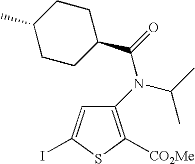

A solution of Intermediate 21 [methyl 3-[[(trans-4-methylcyclohexyl)carbonyl](1-methylethyl)amino]-2-thiophenecarboxylate] (10.5 g) in dry THF (100 mL) was added dropwise at −77° C. under nitrogen to a 2M solution of LDA in THF/heptane/ethyl benzene (48.4 mL) maintaining an internal temperature <−70° C. The dropping funnel was washed through with dry THF (10 mL) and stirring continued at −77° C. for 2½ hours. A solution of iodine (16.5 g) in dry THF (100 mL) was added dropwise to the stirred reaction mixture maintaining an internal temperature <−70° C., then the dropping funnel was washed through with dry THF (10 m). After stirring under nitrogen at −77° C. for 1½ hours, the reaction mixture was quenched by addition of saturated ammonium chloride solution and warmed to 0° C. The mixture was diluted with 5% sodium thiosulfate solution, then the organic phase was separated and the aqueous phase was extracted with EtOAc. The combined organic phases were dried (Na₂SO₄), filtered and evaporated. The crude product was purified by flash chromatography over silica gel (Biotage) eluting with cyclohexane/EtOAc (10:1) to give the title compound.

MS calcd for (C₁₇H₂₄NIO₃S+H)⁺: 450

MS found (electrospray): (M+H)⁺=450

### Intermediate 6

### 2-(4-Bromophenyl)furo[3,2-b]pyridine

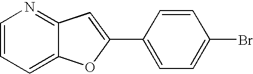

To 4-bromophenylactetylene (4.3 g) was added 2-iodo-3-hydroxypyridine (5.25 g), bis(triphenylphosphine)palladium dichloride (1.5 g), copper (I) iodide (0.58 g) and triethylamine (100 mL). The reaction was stirred at 90° C., under nitrogen, for 4 h. The reaction was cooled, diluted with EtOAc (400 mL) and washed twice with saturated ammonium chloride solution, brine and then concentrated. The crude product was dissolved in DCM, filtered and purified by ISCO companion silica chromatography eluting with a gradient of ethyl acetate in cyclohexane (30-100%) to give the title compound.

MS calcd for (C₁₃H₈BrNO+H)⁺: 274/276

MS found (electrospray): (M+H)⁺=274/276

### Alternative Preparation of Intermediate 6 (Method B)

### 2-(4-Bromophenyl)furo[3,2-b]pyridine

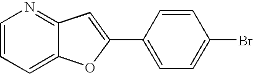

A suspension of bis(triphenylphosphine)palladium dichloride (92 mg) and copper (I) iodide (430 mg) in DMF (7.5 mL) was treated with triethylamine (4.6 mL) and heated to 70° C. 4-Bromophenylacetylene (3.98 g) and 2-iodo-3-hydroxypyridine (6.12 g) were dissolved in DMF (13 mL) and added dropwise to the mixture over 80 min. The mixture was heated at 70° C. for 19 h and cooled to 37° C. Water (45 mL) was added over 10 min and the resulting suspension was filtered, washing with 1:1 DMF:water (2×60 mL) and then water (40 mL). The wet solid was suspended in 3:1 toluene:MeOH (70 mL), heated to reflux for ˜30 mins and cooled. Charcoal (1.13 g) was added and the mixture was refluxed for 1 h. The mixture was cooled, filtered through silica (11.3 g) and washed with 3:1 toluene:MeOH (2×20 mL). The filtrate was concentrated to 80% of its volume and iso-propanol (60 mL) was added. The mixture was further concentrated to 80% of its volume, and the suspension was then allowed to cool to ambient temperature overnight. The mixture was cooled further to 5° C., filtered and washed with cold iso-propanol (2×9 mL). The solid was dried in vacuo to give the title compound.

¹H NMR (d₆-DMSO): δ8.51 (1H, bd), 8.05 (1H, d), 7.93 (2H, d), 7.8-7.7 (3H, m), 7.35 (1H, dd).

### Intermediate 7

### 2-[4-(4,4,5,5-Tetramethyl-1,3,2-dioxaborolan-2-yl)phenyl]furo[3,2-b]pyridine

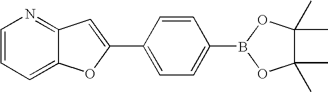

A mixture of Intermediate 6 (250 mg), bis(pinacolato)diboron (345 mg), potassium acetate (265 mg) and 1,1′-bis(diphenylphosphino)ferrocene dichloro palladium (II) (48 mg) in dry dioxane (5 mL) was heated to 100° C. under nitrogen for 7 h. The solvent was evaporated and partitioned between water (10 mL) and DCM (20 mL). The aqueous phase was extracted further with DCM (10 mL) and the combined organics evaporated. The residue was purified by SPE chromatography eluting with cyclohexane/EtOAc (2:1 then 1:1). Further purification by SPE chromatography, eluting with cyclohexane then cyclohexane/EtOAc (3:1) to (1:1) to (1:2) then EtOAc, EtOAc/MeCN (1:1) and finally MeCN gave the title compound.

MS calcd for (C₁₉H₂₀BNO₃+H)⁺: 322

MS found (electrospray): (M+H)⁺=322

### Intermediate 8

### Methyl-5-(4-furo[3,2-b]pyridin-2-ylphenyl)-3-[[(trans-4-methylcyclohexyl)carbonyl](1-methylethyl)amino]-2-thiophenecarboxylate

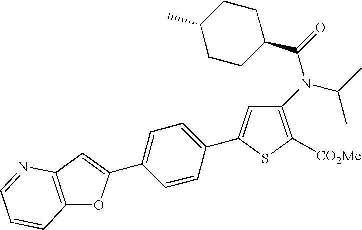

Intermediate 7 (20 mg), methyl 5-iodo-3-[[(trans-4-methylcyclohexyl)carbonyl](1-methylethyl)amino]-2-thiophenecarboxylate (20 mg, a synthesis of which is described above as Intermediate 5) sodium carbonate (19 mg) and tetrakis(triphenylphosphine) palladium (7 mg) in DMF (0.4 mL) were heated in a Reactivial at 100° C. under a nitrogen atmosphere for 4 h. The mixture was evaporated and the crude partitioned between water (10 mL) and DCM (10 mL). The aqueous phase was extracted with DCM (10 mL). The combined organic phases were evaporated and purified by SPE chromatography; eluting with cyclohexane/EtOAc (2:1) to (1:2) to EtOAc to give the title compound.

MS calcd for (C₃₀H₃₂N₂O₄S+H)⁺: 517

MS found (electrospray): (M+H)⁺=517

### Alternative preparation of Intermediate 8 (Method B)

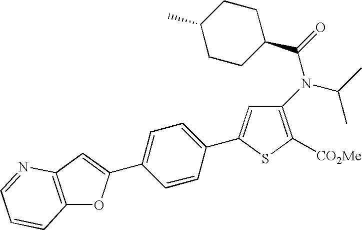

A mixture of {4-[[(trans-4-methylcyclohexyl)carbonyl](1-methylethyl)amino]-5-[(methyloxy)carbonyl]-2-thienyl}boronic acid (6.2 g, 22.6 mmol, a synthesis of which is described as Intermediate 64), 2-(4-bromophenyl)furo[3,2-b]pyridine (8.0 g, 22.8 mmol, a synthesis of which is described above as Intermediate 6), cesium fluoride (33.2 g, 218 mmol), DME (50 mL) and water (50 mL) was stirred vigorously under nitrogen. Pd(PPh₃)₄ (2.48 g, 2.15 mmol) was added and the mixture heated at 85-90° C. for 2 h. After cooling, EtOAc (100 mL) was added and the organic phase washed with portions of water (50 mL), followed by saturated brine, dried (MgSO₄) and evaporated. The residue was dissolved in DCM and purified using ISCO silica chromatography eluting with a gradient of EtOAc in cyclohexane (0-100%) to give the title compound.

MS calcd for (C₃₀H₃₂N₂O₄S+H)⁺: 517

MS found (electrospray): (M+H)⁺=517

### Intermediate 9

### 2-(4-Bromophenyl)pyrazolo[1,5-a]pyrimidine

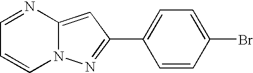

A solution of 3-(4-bromophenyl)-1H-pyrazol-5-amine (5.00 g) in AcOH (80 mL) was treated with 1,1,3,3-tetramethoxypropane (4.13 g) and the mixture heated at 110° C. for 1.5 h. On cooling to room temperature the precipitated solid was isolated by filtration, washed with water (3×10 mL) and dried in vacuo at 40° C. This was recrystallised from acetic acid and dried in vacuo at 40° C. to give the title compound.

MS calcd for (C₁₂H₈BrN₃+H)⁺: 274/276

MS found (electrospray): (M+H)⁺=274/276

### Intermediate 10

### 2-[4-(4,4,5,5-Tetramethyl-1,3,2-dioxaborolan-2-yl)phenyl]pyrazolo[1,5-a]pyrimidine

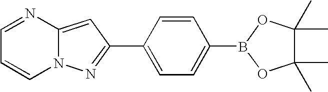

A mixture of Intermediate 9 (100 mg), bis(pinacolato)diboron (134 mg), potassium acetate (108 mg) and 1,1′-bis(diphenylphosphino)ferrocene dichloropalladium (II) (20 mg) in dry dioxane (2 mL) was heated to 100° C. under nitrogen for 15 h. The solvent was evaporated and the residue partitioned between water (10 mL) and DCM (20 mL). The aqueous phase was extracted further with DCM (10 mL) and the combined organics evaporated. This was purified by SPE chromatography, eluting with cyclohexane/EtOAc (3:1) to give the title compound.

MS calcd for (C₁₈H₂₀BN₃O₂+H)⁺: 322

MS found (electrospray): (M+H)⁺=322

### Intermediate 11

### Methyl 3-[[(trans-4-methylcyclohexyl)carbonyl](1-methylethyl)amino]-5-(4-pyrazolo[1,5-a]pyrimidin-2-ylphenyl)-2-thiophenecarboxylate

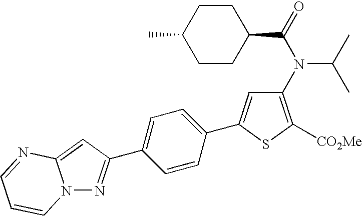

Intermediate 10 (91 mg), methyl 5-iodo-3-[[(trans-4-methylcyclohexyl)carbonyl](1-methylethyl)amino]-2-thiophenecarboxylate (92 mg, a synthesis of which is described above as Intermediate 5), sodium carbonate (1.2 mL) and tetrakis(triphenylphosphine) palladium (44 mg) in DMF (2 mL) were heated at 100° C. under a nitrogen atmosphere for 5 h. The mixture was partitioned between water (10 mL) and DCM (15 mL). The aqueous phase was extracted further with DCM (10 mL). The combined organic phases were evaporated and purified by SPE chromatography eluting with cyclohexane/EtOAc (2:1). Further purification by SPE eluting with DCM to DCM/MeOH (0.5% to 1% MeOH) gave the title compound.

MS calcd for (C₂₉H₃₂N₄O₃S+H)⁺: 517

MS found (electrospray): (M+H)⁺=517

### Alternative preparation of Intermediate 11

### Methyl 3-[[(trans-4-methylcyclohexyl)carbonyl](1-methylethyl)amino]-5-(4-pyrazolo[1,5-a]pyrimidin-2-ylphenyl)-2-thiophenecarboxylate

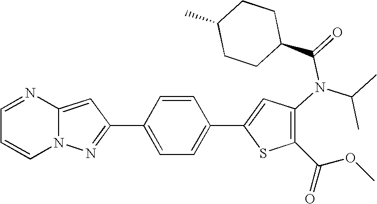

To a mixture of 2-(4-bromophenyl)pyrazolo[1,5-a]pyrimidine (10 g, a synthesis of which is described above as Intermediate 9), {4-[[(trans-4-methylcyclohexyl)carbonyl](1-methylethyl)amino]-5-[(methyloxy)carbonyl]-2-thienyl}boronic acid (14.74 g, a synthesis of which is described above as Intermediate 64) and tetrakis(triphenylphosphine)palladium (0) (4.992 g) was added DME (80 mL) and water (80 mL), and the reaction was stirred at ˜87° C. under nitrogen for 2.25 h. The reaction was then allowed to cool to room temperature and was partitioned between EtOAc and water. The aqueous layer was separated and extracted with EtOAc (×2). The combined organics were washed with water and brine and were dried over sodium sulphate. The mixture was filtered and evaporated in vacuo. The crude material was purified by Biotage silica cartridge, eluting with 2:1 cyclohexane/EtOAc (4000 mL) followed by 1:1 cyclohexane/EtOAc (5000 mL) to give the title compound.

MS calcd for (C₂₉H₃₂N₄O₃S+H)⁺: 517

MS found (electrospray): (M+H)⁺=517

### Intermediate 12

### 2-[4-(4,4,5,5-Tetramethyl-1,3,2-dioxaborolan-2-yl)phenyl]imidazo[1,2-a]pyridine

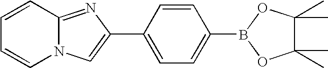

A mixture of 2-(4-bromophenyl)imidazo[1,2-a]pyridine² (300 mg), bis(pinacolato)diboron (508 mg), potassium acetate (393 mg) and 1,1′-bis(diphenylphosphino)ferrocene dichloro palladium (II) (58 mg) in dry dioxane (6 mL) was heated to 100° C. under nitrogen for 24 h. The solvent was evaporated and the residue was partitioned between water (10 mL) and DCM (20 mL). The aqueous phase was extracted further with DCM (10 mL) and the combined organics evaporated. The residue was purified by SPE chromatography eluting with cyclohexane/EtOAc (3:1). Further purification by SPE (silica) eluting with cyclohexane then cyclohexane/EtOAc [(3:1) followed by (1:1)] gave the title compound.

MS calcd for (C₁₉H₂₁BN₂O₂+H)⁺: 321

MS found (electrospray): (M+H)⁺=321

Ref 2: Burkholder, Conrad; Dolbier, William R.; Medebielle, Maurice; Ait-Mohand, Samia, Tetrahedron Lett., 42, 17, 2001, 3077-3080.

### Intermediate 13

### Methyl 5-(4-imidazo[1,2-a]pyridin-2-ylphenyl)-3-[[(trans-4-methylcyclohexyl)carbonyl](1-methylethyl)amino]-2-thiophenecarboxylate

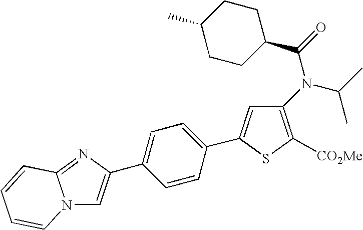

Intermediate 12 (100 mg), methyl 5-iodo-3-[[(trans-4-methylcyclohexyl)carbonyl](1-methylethyl)amino]-2-thiophenecarboxylate (140 mg, a synthesis of which is described above as Intermediate 5), sodium carbonate (133 mg) and tetrakis(triphenylphosphine) palladium (49 mg) in DMF (2 mL) were heated at 100° C. under a nitrogen atmosphere for 17 h. The mixture was partitioned between water (10 mL) and DCM (20 mL). The aqueous phase was extracted further with DCM (10 mL). The combined organic phases were evaporated and purified by SPE chromatography, eluting with cyclohexane/EtOAc (2:1). The crude product was purified by ISCO companion silica chromatography eluting with a gradient of ethyl acetate (0-50%) in cyclohexane to give the title compound.

MS calcd for (C₃₀H₃₃N₃O₃S+H)⁺: 516

MS found (electrospray): (M+H)⁺=516

### Intermediate 14

### 2-(4′-Chloro-4-biphenylyl)-4,4,5,5-tetramethyl-1,3,2-dioxaborolane

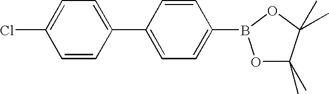

A mixture of 4-chloro-4′-iodobiphenyl (1 g), bis(pinacolato)diboron (971 mg), potassium acetate (935 mg) and 1,1-bis(diphenylphosphino)ferrocene dichloropalladium (II) (254 mg) in dioxane (20 mL) was heated at 100° C. under nitrogen atmosphere for 3 h. The dioxane was evaporated under vacuum and the residue partitioned between DCM and water. The DCM layer was separated using a hydrophobic frit and concentrated. The residue was purified by ISCO Companion chromatography silica chromatography, eluting with a gradient of EtOAc in cyclohexane (0% to 60%) to give the title compound.

MS calcd for (C₁₈H₂₀BClO₂+NH₄)⁺: 332/334

MS found (electrospray): (M+NH₄)⁺=332/334

### Intermediate 15

### Methyl 5-(4′-chloro-4-biphenylyl)-3-[[(trans-4-methylcyclohexyl)carbonyl](1-methylethyl)amino]-2-thiophenecarboxylate

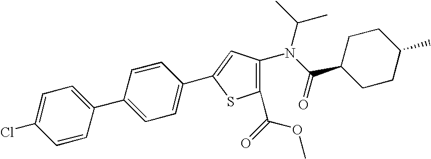

A mixture of Intermediate 14 (126 mg), methyl 5-iodo-3-[[(trans-4-methylcyclohexyl) carbonyl](1-methylethyl)amino]-2-thiophenecarboxylate (150 mg, a synthesis of which is described above as Intermediate 5), 2N sodium carbonate solution (0.7 mL) and tetrakis(triphenylphosphine)palladium (0) (39 mg) in DMF (3 mL) was heated at 100° C. under nitrogen atmosphere, in a Reactivial for 90 minutes. The DMF was evaporated under vacuum and the residue partitioned between DCM and water. The DCM layer was separated using a hydrophobic frit and concentrated. The residue was purified by ISCO Companion silica chromatography, eluting with a gradient of EtOAc in cyclohexane (0% to 40%) to give the title compound.

MS calcd for (C₂₉H₃₂ClNO₃S+H)⁺: 510/512

MS found (electrospray): (M+H)⁺=510/512

### Intermediate 16

### Methyl 5-(4-cyanophenyl)-3-[[(trans-4-methylcyclohexyl)carbonyl](1-methylethyl)amino]-2-thiophenecarboxylate

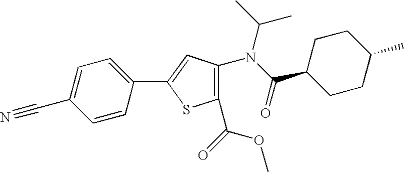

A mixture of (4-cyanophenyl)boronic acid (59 mg), methyl 5-iodo-3-[[(trans-4-methylcyclohexyl)carbonyl](1-methylethyl)amino]-2-thiophenecarboxylate (150 mg, a synthesis of which is described above as Intermediate 5), 2N sodium carbonate solution (0.7 mL) and tetrakis(triphenylphosphine)palladium (0) (39 mg) in DMF (3 mL) was heated at 100° C. under nitrogen atmosphere, in a Reactivial for 90 minutes. The DMF was evaporated under vacuum and the residue partitioned between DCM and water. The DCM layer was separated using a hydrophobic frit and concentrated. The residue was purified by ISCO Companion silica chromatography, eluting with a gradient of EtOAc in cyclohexane (0% to 30%) to give the title compound.

MS calcd for (C₂₄H₂₈N₂O₃S+H)⁺: 425

MS found (electrospray): (M+H)⁺=425

### Intermediate 17

### Methyl 5-(4-acetylphenyl)-3-[[(trans-4-methylcyclohexyl)carbonyl](1-methylethyl)amino]-2-thiophenecarboxylate

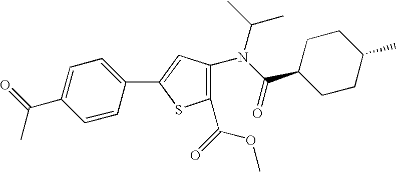

A mixture of (4-acetylphenyl)boronic acid (66 mg), methyl 5-iodo-3-[[(trans-4-methylcyclohexyl)carbonyl](1-methylethyl)amino]-2-thiophenecarboxylate (150 mg, a synthesis of which is described above as Intermediate 5), 2N sodium carbonate solution (0.7 mL) and tetrakis(triphenylphosphine)palladium (0) (39 mg) in DMF (3 mL) was heated at 100° C. under nitrogen atmosphere, in a Reactivial for 90 minutes. The DMF was evaporated under vacuum and the residue partitioned between DCM and water. The DCM layer was separated using a hydrophobic frit and concentrated. The residue was purified by ISCO Companion silica chromatography, eluting with a gradient of EtOAc in cyclohexane (0% to 30%) to give the title compound.

MS calcd for (C₂₅H₃₁NO₄S+H)⁺: 442

MS found (electrospray): (M+H)⁺=442

### Intermediate 18

### Methyl 3-[(1-methylethyl)amino]-5-phenyl-2-thiophenecarboxylate

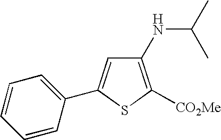

To a solution of 3-amino-5-phenyl-thiophene-2-carboxylic acid methyl ester (4.92 g, 21.1 mmol) in DCM (110 mL) was added 2-methoxypropene (8.23 mL, 84.4 mmol), acetic acid (4.83 mL, 84.4 mmol) and sodium triacetoxyborohydride (8.94 g, 42.2 mmol) and the mixture stirred overnight. Ethyl acetate and water were added, the aqueous phase adjusted to pH7 using sodium bicarbonate and then extracted with ethyl acetate. The combined organic extracts were washed with brine and dried over sodium sulphate. The crude product was purified by silica chromatography using a Flashmaster Personal apparatus (Argonaut) eluting with DCM/cyclohexane (1:4) to give the title compound.

MS calcd for (C₁₅H₁₇NO₂S+H)⁺: 276

MS found (electrospray): (M+H)⁺=276

### Intermediate 19

### Methyl 3-[[(trans-4-methylcyclohexyl)carbonyl](1-methylethyl)amino]-5-phenyl-2-thiophenecarboxylate

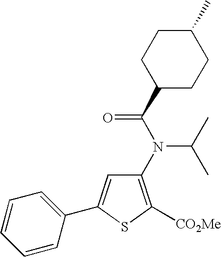

To Intermediate 18 (500 mg, 1.82 mmol) in DCM (4 mL) was added trans-4-methylcyclohexanecarbonyl chloride¹ (350 mg, 2.18 mmol)) followed by triphenylphosphine (500 mg, 1.91 mmol) and the solution stirred at 45° C. for 18 hours. After cooling, it was diluted with ethyl acetate (40 mL) and saturated sodium bicarbonate solution (40 mL). The aqueous phase was washed with ethyl acetate (2×40 mL), the combined organic fractions were dried (Na₂SO₄) and concentrated. The residue was purified by chromatography over silica using a Flashmaster Personal apparatus (Argonaut) eluting with ethyl acetate/cyclohexane (5:95) to give the title compound.

MS calcd for (C₂₃H₂₉NO₃S+H)⁺: 400

MS found (electrospray): (M+H)⁺=400

### Intermediate 20

### Methyl 3-[(1-methylethyl)amino]-2-thiophenecarboxylate

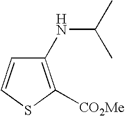

2-Methoxypropene (9.18 mL) was added to a solution of methyl-3-amino-2-thiophene carboxylate (5 g) in dry DCM (100 mL) at room temperature under nitrogen. Glacial acetic acid (5.6 mL) was added slowly. Sodium triacetoxyborohydride (10.12 g) was then added in portions over 30 min. The resulting opaque solution was then left to stir at room temperature for 24 h. The mixture was poured into 8% sodium bicarbonate solution (300 mL), the layers were separated and the DCM layer washed further with bicarbonate solution (2×100 mL), dried (hydrophobic frit) and evaporated to give the title compound.

MS calcd for (C₉H₁₃NO₂S+H)⁺: 200

MS found (electrospray): (M+H)⁺=200

### Alternative preparation of Intermediate 20 (Method B)

### Methyl 3-[(1-methylethyl)amino]-2-thiophenecarboxylate

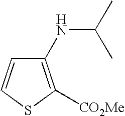

Glacial acetic acid (130 mL) was added dropwise to sodium borohydride (29.5 g) in DCM (600 mL) at 0-5° C. over 50 min. The suspension was stirred at 0-5° C. for 2 h and then warmed at 20-25° C. overnight. A mixture of methyl 3-amino-2-thiophene carboxylate (100 g), 2-methoxy-1-propene (96 mL) and acetic acid (32 mL) in DCM (500 mL) was added dropwise at 20-25° C. over 2 h. Following completion of reaction the mixture was quenched by addition of saturated aqueous sodium bicarbonate (500 mL). The organic phase was separated and the aqueous was extracted with DCM (300 mL). The combined organics were washed with saturated sodium bicarbonate solution (250 mL, then 150 mL) to pH8, water (250 mL) and evaporated in vacuo to give the title compound.

GC (column HP35, 30 mm×0.25 mm×0.25 um, detection temp 250° C., 20 mins run, retention time 5.92 mins)

### Intermediate 21

### Methyl 3-[[(trans-4-methylcyclohexyl)carbonyl](1-methylethyl)amino]-2-thiophenecarboxylate

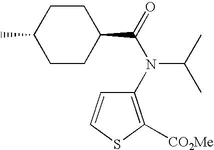

Triphenylphosphine (13.9 g) was added in portions to a solution of methyl 3-[(1-methylethyl)amino]-2-thiophenecarboxylate (6.18 g, a synthesis of which is described above as Intermediate 20) in DCM (dry, 30 mL) at room temperature under nitrogen. trans-4-Methylcyclohexanecarbonyl chloride¹ (8.26 mL) was added in 1 mL portions. The solution was heated to 45° C. under nitrogen for 2 days. A further 2 mL of trans-4-methylcyclohexanecarbonyl chloride¹ was added and heating continued for 24 h. The mixture was cooled, poured into saturated sodium bicarbonate solution and stirred at room temperature to neutralise for 1.5 h. The layers were separated and the aqueous layer was extracted further with DCM (2×100 mL). The combined organic layers were washed with saturated bicarbonate solution (2×100 mL), dried (hydrophobic frit) and evaporated. Cyclohexane (100 mL) was added and the mixture stirred for 1 h, filtered, washed with cyclohexane and air dried to give the title compound.

MS calcd for (C₁₇H₂₅NO₃S+H)⁺: 324

MS found (electrospray): (M+H)⁺=324

Further product was obtained by purification of the filtrate. After evaporation, the residue was applied in the minimum volume of DCM to a 50 g Si SPE cartridge. Elution was with cyclohexane then cyclohexane/ethyl acetate (gradient 9:1 to 8:2). Further purification by 10 g NH2 SPE cartridge, eluting with DCM (5× column volumes), then MeOH (5× column volumes). The DCM fractions were combined and evaporated to give the title compound.

### Alternative preparation of Intermediate 21 (Method B)

### Methyl 3-[[(trans-4-methylcyclohexyl)carbonyl](1-methylethyl)amino]-2-thiophenecarboxylate

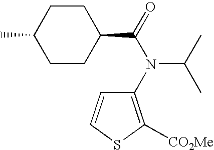

A solution of trans-4-methyl-cyclohexanecarbonyl chloride (182.9 g, a synthesis of which is described below as Intermediate 160) in dichloroethane (650 mL) was added in 4-5 portions at 20-25° C. to a solution of methyl 3-[(1-methylethyl)amino]-2-thiophene carboxylate (127.0 g, a synthesis of which is described above as Intermediate 20) in 1,2-dichloroethane (600 mL) and the mixture was refluxed for 20 h. The mixture was cooled and added portion-wise to saturated sodium bicarbonate solution (750 mL). The organic phase was separated, diluted with 1,2-dichloroethane (100 mL) and water (400 mL). The separated organic phase was washed with saturated sodium bicarbonate solution (500 mL) and water (500 mL). The organic phase was treated with tonsil (5 g) and sodium sulphate (10 g) for 40 min, filtered and evaporated in vacuo. The crude material was recrystallised by dissolution in DCM (600 mL), dilution with heptane (1000 mL) and distillation to remove DCM. The precipitated material was cooled, filtered, washed with heptane (3×100 mL) and dried in vacuo at 30° C. to give the title compound.

¹H NMR (CDCl₃) δ 7.55 (1H, d), 6.85 (1H, d), 4.95 (1H, m), 3.80 (3H, s), 2.0-1.90 (1H, m), 1.80-1.20 (7H, m), 1.1 (3H, d), 0.90 (3H, d), 0.80 (3H, d), 0.7-0.5 (2H, m).

### Intermediate 22

### Methyl 5-(4-chlorophenyl)-3-[(1-methylethyl)amino]-2-thiophenecarboxylate

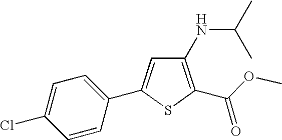

2-Methoxypropene (2.87 mL) was added to a solution of methyl-3-amino-5-(4-chlorophenyl)thiophene-2-carboxylate (2 g) in dry DCM (50 mL) at room temperature under nitrogen. Glacial acetic acid (1.7 mL) was added slowly followed by sodium triacetoxyborohydride (3.17 g) portion wise. The resulting solution was then left to stir at room temperature for 48 h. The mixture was poured into 8% sodium bicarbonate solution, the layers were separated and the aqueous extracted further with DCM (3×140 mL), dried (Na₂SO₄) and evaporated. This was purified by 50 g Si SPE, eluting with cyclohexane then cyclohexane/DCM (3:1 to 1:1 to 1:3) and finally DCM to give the title compound.

MS calcd for (C₁₅H₁₆ClNO₂S+H)⁺: 310/312

MS found (electrospray): (M+H)⁺=310/312

### Intermediate 23

### Methyl 5-(4-chlorophenyl)-3-[[(trans-4-methylcyclohexyl)carbonyl](1-methylethyl)amino]-2-thiophenecarboxylate

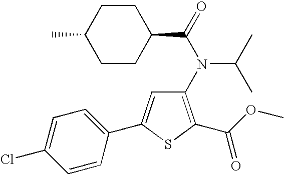

A solution of trans-4-methylcyclohexanecarbonyl chloride¹ (310 mg) in DCM (dry, 3 mL) was added slowly to a solution of Intermediate 22 (500 mg) in DCM (dry, 2 mL) followed by triphenylphosphine (445 mg). The solution was heated to 45° C. under nitrogen for 36 h. On cooling, the mixture was diluted with DCM (20 mL), washed with 8% sodium bicarbonate solution (2×20 mL), water (10 mL), then 2N HCl (2×20 mL), dried (hydrophobic frit) and evaporated. This was purified by 50 g Si SPE (applying in the minimum volume of DCM); eluting with cyclohexane then cyclohexane/DCM (4:1 to 3:2 to 2:3 to 4:1) and finally DCM. Further elution with EtOAc/DCM (1:4 to 2:3 to 3:2) then EtOAc gave the title compound.

MS calcd for (C₂₃H₂₈ClNO₃S+H)⁺: 434/436

MS found (electrospray): (M+H)⁺=434/436

Ref 1: WO 2004/052885

### Intermediate 24

### 6-[4-(4,4,5,5-Tetramethyl-1,3,2-dioxaborolan-2-yl)phenyl]imidazo[2,1-b][1,3]thiazole

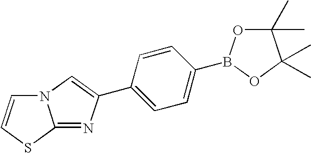

6-(4-Iodophenyl)imidazo[2,1-b][1,3]thiazole (2 g) was dissolved in dry 1,4-dioxane (40 mL). To this stirred solution was added potassium acetate (1.8 g), bis(pinacolato)diboron (2.34 g) and 1,1′-bis(diphenylphosphino)ferrocene dichloro palladium (II) (350 mg). The reaction mixture was then heated to 100° C., and stirred under nitrogen for 18 h. Further, potassium acetate (1.8 g), bis(pinacolato) diboron (2.3 g) and 1,1′-bis(diphenylphosphino)ferrocene dichloropalladium (II) (350 mg) was added. The reaction mixture was then left to stir at 100° C. for a further 24 h. The solvent was then removed by evaporation and the residue was partitioned between water and DCM. The layers were separated using a hydrophobic frit, and the organic phase was concentrated by evaporation to give a solid. This was purified by ISCO companion silica chromatography eluting with a gradient of EtOAc/cyclohexane (10% to 50%), to give the title compound.

MS calcd for (C₁₇H₁₉BN₂O₂S+H)⁺: 327

MS found (electrospray): (M+H)⁺=327

### Intermediate 25

### Methyl 5-(4-imidazo[2,1-b][1,3]thiazol-6-ylphenyl)-3-[[(4-methylcyclohexyl)carbonyl](1-methylethyl)amino]-2-thiophenecarboxylate

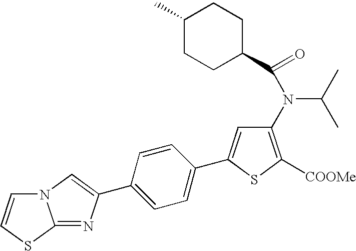

Methyl 5-iodo-3-[[(trans-4-methylcyclohexyl)carbonyl](1-methylethyl)amino]-2-thiophene carboxylate (500 mg, a synthesis of which is described above as Intermediate 5), was added to Intermediate 24 (508 mg), sodium carbonate (471 mg) (dissolved in a minimum volume of water before addition) and Pd(PPh₃)₄ (173 mg) in dry DMF (45 mL). The reaction mixture was then heated to 100° C. under nitrogen with stirring for 3 h. The reaction mixture was allowed to cool, and the DMF was removed by evaporation. The residue was then partitioned between water and DCM, and the layers separated using a hydrophobic frit. The organic phase was then concentrated by evaporation to give an oil. This was then purified by ISCO companion silica chromatography eluting with a gradient of EtOAc/cyclohexane (20% to 80%), to give the title compound.

MS calcd for (C₂₈H₃₁N₃O₃S₂+H)⁺: 522

MS found (electrospray): (M+H)⁺=522

### Intermediate 26

### 2-(4-Bromophenyl)-5-methylpyrazolo[1,5-a]pyrimidin-7-amine

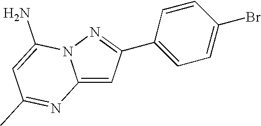

5-Amino-3-(4-bromophenyl)pyrazole (5 g) was dissolved in acetic acid (80 mL) and 3-aminocrotonitrile (1.7 g) was added. The reaction mixture was stirred at 110° C. for 3 h and was then allowed to cool to room temperature. The solvent was evaporated in vacuo and the residue taken up in saturated sodium bicarbonate solution. The solid was then filtered off, washed with water and isopropanol and dried in a vacuum oven at 40° C. to give the title compound.

MS calcd for (C₁₃H₁₁N₄Br+H)⁺: 303/305

MS found (electrospray): (M+H)⁺=303/305

### Intermediate 27

### 5-Methyl-2-[4-(4,4,5,5-tetramethyl-1,3,2-dioxaborolan-2-yl)phenyl]pyrazolo[1,5-a]pyrimidin-7-amine

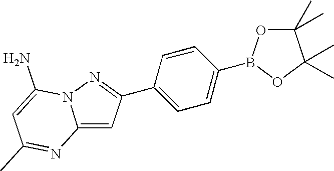

Intermediate 26 (1.5 g) was dissolved in dry 1,4-dioxane (30 mL). To this stirred solution was added potassium acetate (1.36 g), bis(pinacolato)diboron (1.63 g) and 1,1′-bis (diphenylphosphino)ferrocene dichloro palladium (II) (243 mg). The reaction mixture was then heated to 100° C., and stirred under nitrogen for 24 h. After cooling, potassium acetate (1.63 g), bis(pinacolato) diboron (1.36 g) and PdCl₂(dppf)₂ (242 mg) were added and the reaction mixture was then stirred at 100° C. for a further 24 h under nitrogen. The reaction mixture was then cooled and treated with potassium acetate (1.36 g), bis(pinacolato)diboron (1.63 g) and 1,1′-bis(diphenylphosphino)ferrocene dichloro palladium (II) (243 mg). The reaction mixture was heated to 100° C., and stirred under nitrogen for 16 h. The solvent was then removed by evaporation and the residue was partitioned between water and DCM. The layers were separated using a hydrophobic frit, and the organic phase was concentrated by evaporation to give a solid. This was purified using by ISCO companion silica chromatography eluting with a gradient of EtOAc/cyclohexane (10% to 100%). The product was further purified by ISCO companion silica chromatography eluting with a gradient of EtOAc/cyclohexane (20% to 100%), to give the title compound.

MS calcd for (C₁₉H₂₃BN₄O₂+H)⁺: 327

MS found (electrospray): (M+H)⁺=327

### Intermediate 28

### Methyl 5-[4-(7-amino-5-methylpyrazolo[1,5-a]pyrimidin-2-yl)phenyl]-3-[[(4-methylcyclohexyl)carbonyl](1-methylethyl)amino]-2-thiophenecarboxylate

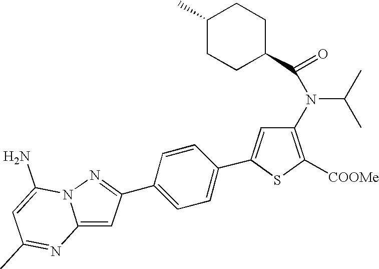

Methyl 5-iodo-3-[[(trans-4-methylcyclohexyl)carbonyl](1-methylethyl)amino]-2-thiophene carboxylate (316 mg, a synthesis of which is described above as Intermediate 5), was added to Intermediate 27 (370 mg), sodium carbonate solution (3.87 ml, 1 M), and Pd(PPh₃)₄ (114 mg) in dry DMF (10 mL), and the reaction mixture was heated to 100° C. under nitrogen with stirring for 5 h. The reaction mixture was allowed to cool, and the DMF was removed by evaporation. The residue was partitioned between water and EtOAc. The organic layer was evaporated to a foam and was purified by ISCO companion silica chromatography eluting with a gradient of EtOAc/cyclohexane (5% to 100%), to give the title compound.

MS calcd for (C₃₀H₃₅N₅O₃S+H)⁺: 546

MS found (electrospray): (M+H)⁺=546

### Intermediate 29

### 2-(4-Bromophenyl)-5-methylpyrazolo[1,5-a]pyrimidine

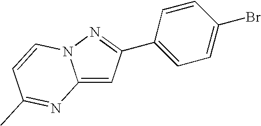

5-Amino-3-(4-bromophenyl)pyrazole (5 g) was dissolved in dry ethanol (100 mL) and 4,4-dimethoxy-2-butanone (2.7 mL) was added. The reaction was refluxed for 18 h under nitrogen, and was then allowed to cool to room temperature. A precipitate was filtered off and dried in a vacuum oven at 40° C. for 18 h to give the title compound.

MS calcd for (C₁₃H₁₀N₃Br+H)⁺: 288/290

MS found (electrospray): (M+H)⁺=288/290

### Intermediate 30

### 5-Methyl-2-[4-(4,4,5,5-tetramethyl-1,3,2-dioxaborolan-2-yl)phenyl]pyrazolo[1,5-a]pyrimidine

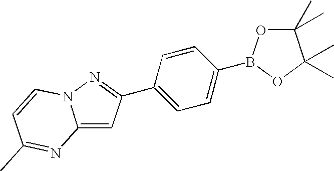

Intermediate 29 (1.8 g) was dissolved in dry 1,4-dioxane (30 mL). To this stirred solution was added potassium acetate (1.72 g), bis(pinacolato)diboron (2.06 g) and 1,1′-bis (diphenylphosphino)ferrocene dichloro palladium (II) (306 mg). The reaction mixture was then heated to 100° C., and stirred under nitrogen for 48 h. The solvent was removed by evaporation and the residue was partitioned between water and DCM. The layers were separated, the organic layer filtered, and the organic phase was concentrated by evaporation to give the title compound.

MS calcd for (C₁₉H₂₂BN₃O₂+H)⁺: 336

MS found (electrospray): (M+H)⁺=336

### Alternative Preparation of Intermediate 30 (Method B)

### 5-Methyl-2-[4-(4,4,5,5-tetramethyl-1,3,2-dioxaborolan-2-yl)phenyl]pyrazolo[1,5-a]pyrimidine

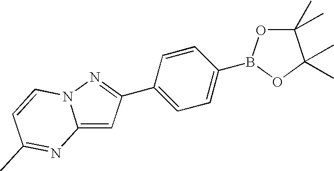

2-(4-Bromophenyl)-5-methylpyrazolo[1,5-a]pyrimidine (100 mg, a synthesis of which is described above as Intermediate 29), potassium acetate (102 mg), bis(pinacolato)diboron (89 mg) and 1,1′-bis(diphenylphosphino)ferrocene dichloro palladium (II) (28 mg) were dissolved in 1,4-dioxane (1 mL) and were heated in a microwave at 120° C. for 35 mins. The reaction was evaporated in vacuo and was partitioned between DCM and water. The aqueous layer was extracted with DCM, and the combined organics were evaporated in vacuo to give the title compound.

MS calcd for (C₁₉H₂₂BN₃O₂+H)⁺: 336

MS found (electrospray): (M+H)⁺=336

### Intermediate 31

### Methyl 3-[[(4-methylcyclohexyl)carbonyl](1-methylethyl)amino]-5-[4-(5-methylpyrazolo[1,5-a]pyrimidin-2-yl)phenyl]-2-thiophenecarboxylate

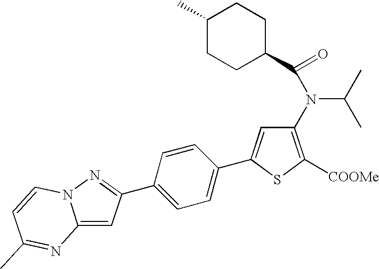

To methyl 5-iodo-3-[[(trans-4-methylcyclohexyl)carbonyl](1-methylethyl)amino]-2-thiophene carboxylate (500 mg, a synthesis of which is described above as Intermediate 5) in dry DMF (10 mL) was added 5-methyl-2-[4-(4,4,5,5-tetramethyl-1,3,2-dioxaborolan-2-yl)phenyl]pyrazolo[1,5-a]pyrimidine (558 mg, a synthesis of which is described above as Intermediate 30), sodium carbonate (6.1 mL, 1 M), and Pd(PPh₃)₄ (180 mg) and the reaction mixture was heated to 100° C. under nitrogen with stirring for 2 h. The reaction mixture was allowed to cool, and the DMF was removed by evaporation. The residue was then partitioned between water and EtOAc, the organic layer was separated, dried over sodium sulphate, filtered and evaporated. The residue was purified by ISCO companion silica chromatography eluting with a gradient of EtOAc/cyclohexane (5% to 100%), to give the title compound.

MS calcd for (C₃₀H₃₄N₄O₃S+H)⁺: 531

MS found (electrospray): (M+H)⁺=531

### Alternative Preparation of Intermediate 31 (Method B)

### Methyl 3-[[(4-methylcyclohexyl)carbonyl](1-methylethyl)amino]-5-[4-(5-methylpyrazolo[1,5-a]pyrimidin-2-yl)phenyl]-2-thiophenecarboxylate

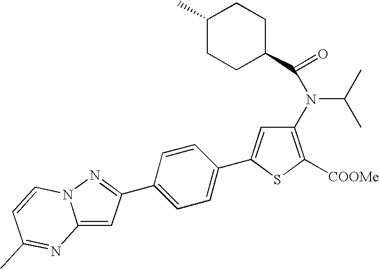

A mixture of {4-[[(trans-4-methylcyclohexyl)carbonyl](1-methylethyl)amino]-5-[(methyloxy) carbonyl]-2-thienyl}boronic acid (15.9 g, 43.3 mmol, a synthesis of which is described as Intermediate 64), 2-(4-bromophenyl)-5-methylpyrazolo[1,5-a]pyrimidine (11.3 g, 39.3 mmol, a synthesis of which is described as Intermediate 29), cesium fluoride (29.85 g, 197 mmol), DME (90 mL), water (90 mL) and Pd(PPh₃)₄ (5.45 g, 4.72 mmol) was heated at 87° C. with stirring under nitrogen for 4 h. After cooling, the mixture was partitioned between EtOAc and water. The organic layer was washed with water and brine. The aqueous phase was further washed with EtOAc. The combined organic fractions were dried (Na₂SO₄) and evaporated in vacuo. The residue was dissolved in warm ethanol (approx 50° C.) and allowed to cool. The resulting crystals were washed with cold ether to afford the crude product. The mother liquors were evaporated, combined with the crude product, dissolved in the minimum volume of DCM and purified using ISCO silica chromatography eluting with a gradient of EtOAc in cyclohexane (5-100%) to give the title compound.

MS calcd for (C₃₀H₃₄N₄O₃S+H)⁺: 531

MS found (electrospray): (M+H)⁺=531

### Intermediate 32

### 2-(4-Bromophenyl)pyrazolo[1,5-a]pyrimidin-7-amine

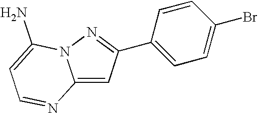

5-Amino-3-(4-bromophenyl)pyrazole (5 g) was dissolved in acetic acid (80 mL) and trans-3-(dimethylamino)-acrylonitrile (2.3 mL) was added. The reaction heated to 110° C. for 3 h, and was then allowed to cool to room temperature before being evaporated in vacuo. The residue was treated with saturated sodium bicarbonate solution and the resulting precipitate was filtered off, washed with water and dried in a vacuum oven for 18 h to give the title compound.

MS calcd for (C₁₂H₉N₄Br+H)⁺: 289/291

MS found (electrospray): (M+H)⁺=289/291

### Intermediate 33

### 2-[4-(4,4,5,5-Tetramethyl-1,3,2-dioxaborolan-2-yl)phenyl]pyrazolo[1,5-a]pyrimidin-7-amine

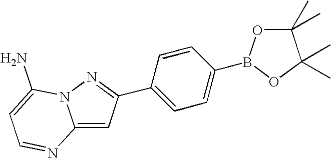

Intermediate 32 (1.5 g) was dissolved in dry 1,4-dioxane (30 mL). To this stirred solution was added potassium acetate (1.42 g), bis(pinacolato)diboron (1.71 g) and 1,1′-bis (diphenylphosphino)ferrocene dichloro palladium (II) (254 mg). The reaction mixture was heated to 100° C., and stirred under nitrogen for 18 h. The reaction was then cooled and recharged with potassium acetate (1.42 g), bis(pinacolato)diboron (1.713 g) and 1,1′-bis (diphenylphosphino)ferrocene dichloro palladium (II) (254 mg). The reaction mixture was then reheated to 100° C., and stirred under nitrogen for another 18 h. The reaction was allowed to cool and the solvent removed by evaporation and the residue was partitioned between water and DCM. The organic layer was separated, washed with water, and concentrated by evaporation. The residue was then purified by ISCO companion silica chromatography eluting with a gradient of EtOAc/cyclohexane (0% to 100%), to give the title compound.

MS calcd for (C₁₈H₂₁BN₄O₂+H)⁺: 337

MS found (electrospray): (M+H)⁺=337

### Intermediate 34

### Methyl 5-[4-(7-aminopyrazolo[1,5-a]pyrimidin-2-yl)phenyl]-3-[[(4-methylcyclohexyl)carbonyl](1-methylethyl)amino]-2-thiophenecarboxylate

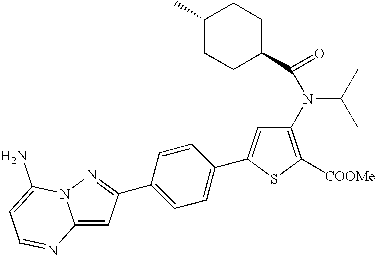

To methyl 5-iodo-3-[[(trans-4-methylcyclohexyl)carbonyl](1-methylethyl)amino]-2-thiophene carboxylate (500 mg, a synthesis of which is described above as Intermediate 5) in dry DMF (6 ml) was added Intermediate 33 (562 mg) in dry DMF (4 mL), sodium carbonate (6.1 mL, 1 M), and Pd(PPh₃)₄ (180 mg), and the reaction mixture was heated to 100° C. under nitrogen with stirring for 4.75 h. Then the reaction mixture was allowed to cool, and the DMF was removed by evaporation. The residue was then partitioned between water and DCM. The organic layer was washed with brine, separated, dried over sodium sulphate, filtered and evaporated. The residue was partitioned between EtOAc and water. The organic layer separated, dried over sodium sulphate, filtered and evaporated. This was then purified by ISCO companion silica chromatography eluting with a gradient of EtOAc/cyclohexane (5% to 100%), to give the title compound.

MS calcd for (C₂₉H₃₃N₅O₃S+H)⁺: 532

MS found (electrospray): (M+H)⁺=532

### Intermediate 35

### 5-Iodo-3-[[(4-methylcyclohexyl)carbonyl](1-methylethyl)amino]-2-thiophenecarboxylic acid

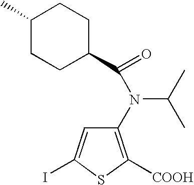

To methyl 5-iodo-3-[[(trans-4-methylcyclohexyl)carbonyl](1-methylethyl)amino]-2-thiophene carboxylate (800 mg, a synthesis of which is described above as Intermediate 5) in methanol (5 mL) and THF (5 mL) was added sodium hydroxide solution (2 N, 5 mL), and the reaction mixture stirred for 2 h. The reaction mixture solvents were removed by evaporation and the residue was partitioned between water and EtOAc. The aqueous phase was then acidified with 2 N hydrochloric acid and extracted with EtOAc. The organic layer was separated, washed with brine, dried over sodium sulphate, filtered and evaporated. This was then purified by ISCO companion silica chromatography eluting with a gradient of methanol/DCM (0% to 70%), to give the title compound.

MS calcd for (C₁₆H₂₂NIO₃S+H)⁺: 436

MS found (electrospray): (M+H)⁺=436

### Intermediate 36

### 2-(4-Bromophenyl)[1,3]oxazolo[4,5-b]pyridine

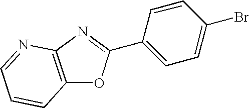

2-Amino-3-hydroxypyridine (1 g) was dissolved in PPA (2.5 g) containing 4-bromobenzoic acid (3.65 g). The reaction was heated to 185° C. for 0.3 h, and was then allowed to cool to room temperature before being treated with ice water to cause precipitation of a solid which was collected by filtration and discarded. The filtrate was washed with saturated sodium bicarbonate solution and extracted with DCM, the organics separated, dried over magnesium sulphate, filtered and evaporated to a solid. This was purified by ISCO companion silica chromatography eluting with a gradient of EtOAc/cyclohexane (0% to 100%), to give the title compound.

MS calcd for (C₁₂H₇N₂BrO+H)⁺: 276/278

MS found (electrospray): (M+H)⁺=276/278

### Intermediate 37

### 2-[4-(4,4,5,5-Tetramethyl-1,3,2-dioxaborolan-2-yl)phenyl][1,3]oxazolo[4,5-b]pyridine

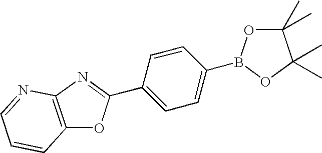

Intermediate 36 (0.88 g) was dissolved in dry 1,4-dioxane (18 mL). To this stirred solution was added potassium acetate (0.95 g), bis(pinacolato)diboron (1.23 g) and 1,1′-bis (diphenylphosphino)ferrocene dichloro palladium (II) (170 mg). The reaction mixture was heated to 100° C., and stirred under nitrogen for 24 h. The reaction was allowed to cool, the solvent removed by evaporation and the residue was partitioned between water and DCM. The organic layer was separated, washed with water, and concentrated by evaporation. The residue was then purified by ISCO companion silica chromatography eluting with a gradient of EtOAc/cyclohexane (0% to 100%), to give the title compound.

MS calcd for (C₁₈H₁₉BN₂O₃+H)⁺: 323

MS found (electrospray): (M+H)⁺=323

### Intermediate 38

### 5-Bromo-3-iodo-2(1H)-pyridinone

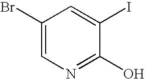

5-Bromo-2(1H)-pyridinone (5 g) and N-iodosuccinimide (6.47 g) were mixed in dry acetonitrile (100 mL) at 90° C. under nitrogen. After 2 h, the mixture was cooled to room temperature and the precipitate filtered off, washed with methanol and dried under vacuum to give the title compound.

MS calcd for (C₅H₃BrINO+H)⁺: 299/301

MS found (electrospray): (M+H)⁺=299/301

### Intermediate 39

### 5-Bromo-3-[(trimethylsilyl)ethynyl]-2(1H)-pyridinone

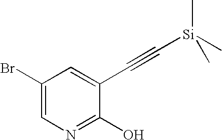

Intermediate 38 (5 g), copper (I) iodide (0.23 g), Pd(PPh₃)₂Cl₂ (0.84 g), trimethylsilyl acetylene (2.36 mL) and triethylamine (11.7 mL) were mixed in dry THF (44 mL). The stirred solution was degassed and placed under a nitrogen atmosphere, and heated at 60° C. for 2.5 h. Solvent was then removed under vacuum. The residue was partitioned between DCM and water. The organic phase was dried by a hydrophobic frit, and the solvent removed under vacuum to give a solid. This was purified by ISCO companion silica chromatography eluting with a gradient of EtOAc/cyclohexane (0% to 60%), and crystallized from hot ethanol and filtered to give the title compound.

MS calcd for (C₁₀H₁₂BrNOSi+H)⁺: 270/272

MS found (electrospray): (M+H)⁺=270/272

### Intermediate 40

### 5-Bromo-3-ethynyl-2(1H)-pyridinone

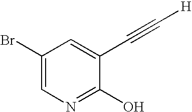

A mixture of Intermediate 39 (900 mg) and potassium carbonate (920 mg) was stirred in ethanol (50 mL) under nitrogen for 18 h. The reaction mixture which contained a solid was evaporated under vacuum. The residue was triturated with water and the solid filtered off. The aqueous filtrate was reduced in volume and purified by ISCO companion C18 chromatography eluted with water (0.1% formic acid) then a gradient of water:acetonitrile (0-100% containing 0.05% formic acid) to give the title compound.

MS calcd for (C₇H₄BrNO+H)⁺: 198/200

MS found (electrospray): (M+H)⁺=198/200

### Intermediate 41

### 5-Bromofuro[2,3-b]pyridine

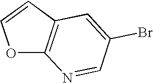

Intermediate 40 (282 mg) was dissolved in DMSO (5 mL) and was heated by microwave radiation to 120° C. for 0.5 h. The solution was partitioned between DCM and water. The aqueous layer was extracted with DCM, the organics combined and washed with water and evaporated under vacuum. The residual oil was purified by reverse phase SPE chromatography, eluting with water, then methanol. The product fractions were evaporated to an oil which crystallised on standing and dried under high vacuum to give the title compound.

¹H NMR (CDCl₃): δ8.38 (1H, d), 8.075 (1h, d), 7.735 (1H, d), 6.765 (1H, d).

### Intermediate 42

### Methyl 5-(4-bromophenyl)-3-[[(4-methylcyclohexyl)carbonyl](1-methylethyl)amino]-2-thiophenecarboxylate

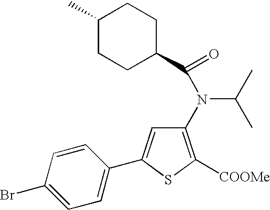

Methyl 5-iodo-3-[[(trans-4-methylcyclohexyl)carbonyl](1-methylethyl)amino]-2-thiophene carboxylate (1 g, a synthesis of which is described above as Intermediate 5) was added to 4-bromophenylboronic acid (420 mg), sodium carbonate solution (2 N, 4.4 mL), and Pd(PPh₃)₄ (250 mg) in dry DMF (20 mL), and the reaction mixture was then heated to 100° C. under nitrogen with stirring for 4 h. The reaction mixture was allowed to cool, and the DMF was removed by evaporation. The residue was then partitioned between water and EtOAc. The organic phase was separated, dried over sodium sulphate, filtered and evaporated under vacuum. This was purified by ISCO companion silica chromatography eluting with a gradient of EtOAc/cyclohexane (0% to 50%), and further purification carried out by crystallisation from ether to give the title compound.

MS calcd for (C₂₃H₂₈NBrO₃S+H)⁺: 478/480

MS found (electrospray): (M+H)⁺=478/480

### Intermediate 43

### Methyl 3-[[(4-methylcyclohexyl)carbonyl](1-methylethyl)amino]-5-[4-(4,4,5,5-tetramethyl-1,3,2-dioxaborolan-2-yl)phenyl]-2-thiophenecarboxylate

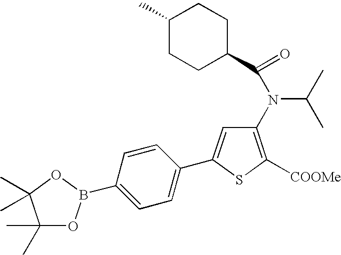

Intermediate 42 (210 mg) was dissolved in dry 1,4-dioxane (4 mL). To this stirred solution was added potassium acetate (151 mg), bis(pinacolato)diboron (179 mg) and 1,1′-bis (diphenylphosphino)ferrocene dichloro palladium (II) (25 mg). The reaction mixture was then heated to 100° C., and stirred under nitrogen for 2.5 h. The reaction was allowed to cool and the solvent removed by evaporation and the residue was partitioned between water and DCM. The organic layer was separated, and concentrated by evaporation to give a solid. This was purified by SPE silica chromatography eluting with cyclohexane, then cyclohexane/DCM (2:1), then diethylether, then ethyl acetate to give the title compound.

MS calcd for (C₂₉H₄₀BNO₅S+H)⁺: 526

MS found (electrospray): (M+H)⁺=526

### Intermediate 44

### Methyl 5-(4-furo[2,3-b]pyridin-5-ylphenyl)-3-[[(4-methylcyclohexyl)carbonyl](1-methyl ethyl)amino]-2-thiophenecarboxylate

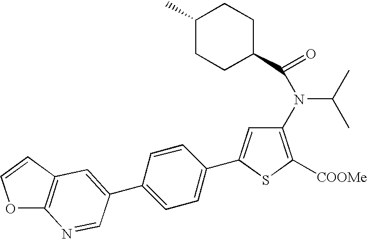

To Intermediate 43 (240 mg) in dioxane/water (3:1, 4 mL) was added Intermediate 41 (143 mg), sodium carbonate (195 mg), and Pd(PPh₃)₄ (49 mg), and the reaction mixture was then heated to 100° C. with microwave radiation for 0.3 h. The reaction mixture was allowed to cool and was then partitioned between DCM/water. The organic layer was separated using a hydrophobic frit and evaporated to a solid. This was purified by ISCO companion silica chromatography eluting with a gradient of EtOAc/cyclohexane (0% to 60%), to give the title compound.

MS calcd for (C₃₀H₃₂N₂O₄S+H)⁺: 517

MS found (electrospray): (M+H)⁺=517

### Intermediate 45

### 2-(4-Bromophenyl)-5-nitro-1,3-benzoxazole

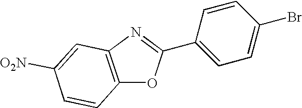

2-Amino-4-nitrophenol (278 mg) was dissolved in dioxan (2.5 mL) containing 4-bromobenzoyl chloride (439 mg). The reaction heated to 210° C. with microwave radiation for 0.25 h, and was then allowed to cool to room temperature before being added to sodium hydroxide (50 mL, 1 M). A precipitate resulted which was filtered off and dried under reduced pressure to give the title compound.

MS calcd for (C₁₃H₇N₂BrO₃+H)⁺: 319/321

MS found (electrospray): (M+H)⁺=319/321

### Intermediate 46

### 2-(4-Bromophenyl)-1,3-benzoxazol-5-amine

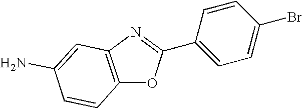

Intermediate 45 (465 mg) and tin (II) chloride (640 mg) was dissolved in THF (25 mL) and stirred at room temperature. Concentrated HCl (10 drops) was added and the reaction mixture stirred for 5 h. More tin (II) chloride (640 mg) and concentrated HCl was added and the reaction stirred for a further 18 h. Further tin (II) chloride (640 mg) and concentrated HCl (5 drops) were added and the reaction stirred for a further 5 h. The reaction mixture was diluted with water (100 mL) and basified with 2M sodium hydroxide. This solution was then extracted with EtOAc. The organic layer was separated, dried over magnesium sulphate, filtered and evaporated to a solid. This was purified by ISCO companion silica chromatography eluting with a gradient of EtOAc/cyclohexane (0% to 100%) to give the title compound.

MS calcd for (C₁₉H₂₁BN₂O₃+H)⁺: 289/291

MS found (electrospray): (M+H)⁺=289/291

### Intermediate 47

### 2-[4-(4,4,5,5-Tetramethyl-1,3,2-dioxaborolan-2-yl)phenyl]-1,3-benzoxazol-5-amine

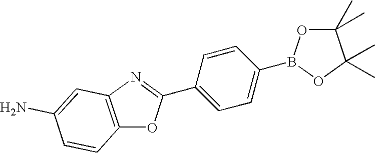

Intermediate 46 (600 mg) was dissolved in dry 1,4-dioxane (12 mL). To this stirred solution was added potassium acetate (615 mg), bis(pinacolato)diboron (790 mg) and 1,1′-bis (diphenylphosphino)ferrocene dichloro palladium (II) (112 mg). The reaction mixture was then heated to 100° C., and stirred under nitrogen for 4 h. The reaction was allowed to cool and the solvent removed by evaporation and the residue was partitioned between water and DCM. The organic layer was passed through a hydrophobic frit, and concentrated by evaporation to give a solid. This was purified by ISCO companion silica chromatography eluting with a gradient of EtOAc/cyclohexane (0% to 100%), to give the title compound.

MS calcd for (C₁₉H₂₁BN₂O₃+H)⁺: 337

MS found (electrospray): (M+H)⁺=337

### Intermediate 48

### Methyl 5-[4-(5-amino-1,3-benzoxazol-2-yl)phenyl]-3-[[(4-methylcyclohexyl)carbonyl](1-methylethyl)amino]-2-thiophenecarboxylate

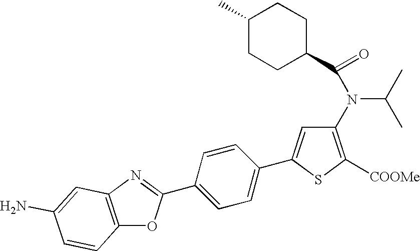

To methyl 5-iodo-3-[[(trans-4-methylcyclohexyl)carbonyl](1-methylethyl)amino]-2-thiophene carboxylate (500 mg, a synthesis of which is described above as Intermediate 5) in dry DMF (8 mL) was added Intermediate 47 (600 mg), sodium carbonate solution (4.45 ml, 2N), and Pd(PPh₃)₄ (180 mg), and the reaction mixture was then heated to 100° C. under nitrogen with stirring for 2 h. The reaction mixture was allowed to cool, and the DMF was removed by evaporation. The residue was then partitioned between water and EtOAc. The organic layer was separated, washed with brine, dried over magnesium sulphate, filtered and evaporated. This was purified by ISCO companion silica chromatography eluting with a gradient of EtOAc/cyclohexane (5% to 100%), to give the title compound.

MS calcd for (C₃₀H₃₃N₃O₄S+H)⁺: 532

MS found (electrospray): (M+H)⁺=532

### Intermediate 49

### 4-Bromo-N-(2-oxo-1,2-dihydro-3-pyridinyl)benzamide

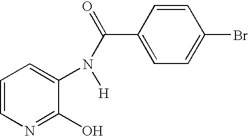

A solution of amino-2(1H)-pyridinone (2.55 g) in pyridine (40 mL) at 21° C. was treated with 4-bromobenzoyl chloride (6.12 g). The reaction was then stirred for 24 h and the reaction mixture was poured onto ice water. A precipitate resulted which was filtered off, washed with water and dried under reduced pressure to give the title compound.

MS calcd for (C₁₂H₉N₂BrO₃+H)⁺: 293/295

MS found (electrospray): (M+H)⁺=293/295

### Intermediate 50

### 2-(4-Bromophenyl)[1,3]oxazolo[5,4-b]pyridine

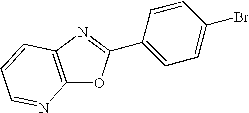

Intermediate 49 (6.7 g) was dissolved in phosphorous oxychloride (50 mL). The reaction heated to reflux for 2 h, and was then allowed to cool to room temperature before being evaporated under vacuum. The residue was azeotroped with toluene to give a solid which was treated with ice water, filtered, washed with water and dried in vacuum to give a solid. This was purified by ISCO companion silica chromatography eluting with a gradient of EtOAc/cyclohexane (0% to 60%), to give the title compound.

MS calcd for (C₁₂H₇N₂BrO+H)⁺: 275/277

MS found (electrospray): (M+H)⁺=275/277

### Intermediate 51

### 2-[4-(4,4,5,5-Tetramethyl-1,3,2-dioxaborolan-2-yl)phenyl][1,3]oxazolo[5,4-b]pyridine

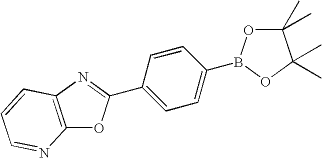

Intermediate 50 (2 g) was dissolved in dry 1,4-dioxane (40 mL). To this stirred solution was added potassium acetate (2.14 g), bis(pinacolato)diboron (2.77 g) and PdCl₂(dppf)₂ (392 mg). The reaction mixture was heated to reflux, and stirred under nitrogen for 2 h. The reaction was allowed to cool and the solvent removed by evaporation and the residue was partitioned between water and DCM. The organic layer was separated and the aqueous layer re-extracted with DCM. The organic extracts were combined, washed with brine, dried over sodium sulphate and concentrated by evaporation to a gum. The residue was purified by ISCO companion silica chromatography eluting with a gradient of EtOAc/cyclohexane (0% to 100%), to give the title compound.

MS calcd for (C₁₈H₁₉BN₂O₃+H)⁺: 323

MS found (electrospray): (M+H)⁺=323

### Intermediate 52

### 2-[4-(4,4,5,5-Tetramethyl-1,3,2-dioxaborolan-2-yl)phenyl][1,3]furo[3,2-c]pyridine

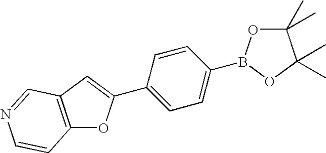

2-(4-Bromophenyl)[1,3]furo[3,2-c]pyridine (783 mg)* was dissolved in dry 1,4-dioxane (30 mL). To this stirred solution was added potassium acetate (839 mg), bis(pinacolato)diboron (1.09 g) and 1,1′-bis(diphenylphosphino)ferrocene dichloro palladium (II) (153 mg). The reaction mixture was heated at 100° C., and stirred under nitrogen for 4 h. The reaction was allowed to cool and the solvent removed by evaporation and the residue was partitioned between water and DCM. The organic layer was separated and the aqueous layer re-extracted with DCM. The organic layers were combined, passed through a hydrophobic frit and concentrated by evaporation to give a residue. This was purified by SPE silica chromatography eluting with a gradient of EtOAc/cyclohexane (10% to 50%), to give the title compound.

MS calcd for (C₁₈H₁₉BN₂O₃+H)⁺: 323

MS found (electrospray): (M+H)⁺=323

Ref: *Collection of Czechoslovak Chemical Communications (1996) 61 (11) p 1627-1636.

### Intermediate 53

### Methyl 5-(4-furo[3,2-c]pyridin-2-ylphenyl)-3-[[(4-methylcyclohexyl)carbonyl](1-methylethyl)amino]-2-thiophenecarboxylate

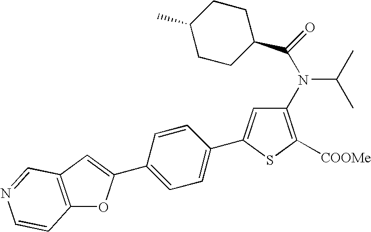

To methyl 5-iodo-3-[[(trans-4-methylcyclohexyl)carbonyl](1-methylethyl)amino]-2-thiophene carboxylate (500 mg, a synthesis of which is described above as Intermediate 5) in dry DMF (10 mL) was added Intermediate 52 (500 mg), sodium carbonate solution (2 M, 6.1 mL), and Pd(PPh₃)₄ (179 mg), and the reaction mixture was heated to 100° C. under nitrogen with stirring for 2 h. The reaction mixture was allowed to cool, and the DMF was removed by evaporation. The residue was then partitioned between 8% sodium carbonate solution and DCM. The aqueous was then re-extracted with DCM. These organic layers were combined, washed with 8% sodium bicarbonate solution, separated, passed through a hydrophobic frit, and evaporated to an oil. This was purified by SPE silica chromatography eluting with a gradient of EtOAc/cyclohexane (0% to 90%), to give the title compound.

MS calcd for (C₃₀H₃₂N₂O₄S+H)⁺: 517

MS found (electrospray): (M+H)⁺=517

### Intermediate 54

### Methyl 3-[(1-methylethyl)(trifluoroacetyl)amino]-2-thiophenecarboxylate

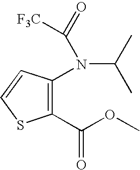

Methyl 3-[(1-methylethyl)amino]-2-thiophenecarboxylate (9.1 g, a synthesis of which is described as Intermediate 20) was dissolved in Et₂O (180 mL) and was cooled to 0° C. Trifluoroacetic anhydride (9.5 mL) was added and the reaction mixture was stirred for 1 h before being warmed to room temperature. The reaction mixture was concentrated in vacuo and was azeotroped with toluene (×3) to give the title compound.

MS calcd for (C₁₁H₁₂F₃NO₃S+H)⁺: 296

MS found (electrospray): (M+H)⁺=296

### Intermediate 55

### Methyl 5-(4-imidazo[1,2-a]pyridin-2-ylphenyl)-3-[(1-methylethyl)amino]-2-thiophenecarboxylate

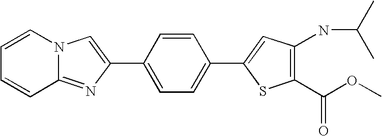

Intermediate 4 (2.0 g, prepared by method B), was dissolved in DMF (180 mL). 2-[4-(4,4,5,5-Tetramethyl-1,3,2-dioxaborolan-2-yl)phenyl]imidazo[1,2-a]pyridine (2.8 g, a synthesis of which is described above as Intermediate 12), sodium carbonate solution (2.6 g in 15 mL H₂O) and tetrakis(triphenylphosphine)palladium (0) (955 mg) were added and the reaction mixture was stirred at 100° C. for 3 h. The reaction mixture was concentrated in vacuo and was partitioned between water and DCM. The aqueous phase was washed with DCM (×3) and the combined organics were passed through a hydrophobic frit and concentrated in vacuo. The crude material was purified by ISCO Companion silica chromatography eluting with a gradient 5-100% EtOAc in cyclohexane to give the title compound.

MS calcd for (C₂₂H₂₁N₃O₂S+H)⁺: 392

MS found (electrospray): (M+H)⁺=392

### Intermediate 56

### 2-(4-Bromophenyl)-6-nitro-1,3-benzoxazole

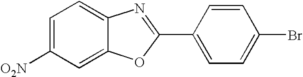

A mixture of 2-amino-5-nitrophenol (278 mg) and 4-bromobenzoyl chloride (439 mg) in dry 1,4-dioxane (2.5 mL) was stirred and heated at 210° C. in a microwave for 15 mins. This was repeated 4 more times using fresh starting materials. The mixtures from all 5 reactions were combined and slowly added to a vigorously stirred 1 M sodium hydroxide solution (250 mL). The precipitate was collected by filtration, washed with water and dried in a vacuum oven to give the title compound.

¹H NMR (CDCl₃) δ 8.51 (1H, d), 8.36 (1H, dd), 8.17 (2H, dt), 7.87 (1H, d), 7.74 (2H, dt).

### Intermediate 57

### trans-4-(Trifluoromethyl)cyclohexanecarbonyl chloride

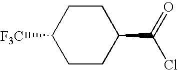

Oxalyl chloride (4.59 mL) was added dropwise to a solution of trans-4-(trifluoromethyl)cyclohexanecarboxylic acid¹ (6.85 g) in dry DCM (100 mL) at room temperature under nitrogen. After 10 mins an effervescence was observed and the reaction was stirred at room temperature overnight. The solvent was evaporated in vacuo to give the title compound.

¹H NMR (d₆-DMSO) δ 2.38-2.19 (2H, m), 1.92 (4H, dd), 1.44-1.22 (4H, m).

Ref 1: DE 39 30 119 (A1)

### Intermediate 58

### 1-(4-Bromophenyl)ethanone oxime (8:1 mixture of E and Z isomers)

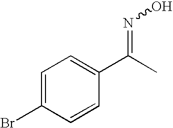

A mixture of 1-(4-bromophenyl)ethanone (5.0 g) and hydroxylamine hydrochloride (1.75 g) in pyridine (10 mL) was stirred at room temperature for 3 h. The mixture was poured into 2M HCl (250 mL) and was extracted with EtOAc (2×250 mL). The combined organics were washed with water (10 mL) and brine (10 mL) and were dried over magnesium sulphate and evaporated in vacuo to give the title compound (as an 8:1 mixture of E/Z isomers).

¹H NMR (d₆-DMSO) δ 11.34 (major, s)+10.79 (minor, s; total both=1H), 7.59 (m, 4H), 3.34 (3H, s).

### Intermediate 59

### Methyl 3-((1-methylethyl){[trans-4-(trifluoromethyl)cyclohexyl]carbonyl}amino)-5-(4-pyrazolo[1,5-a]pyrimidin-2-ylphenyl)-2-thiophenecarboxylate

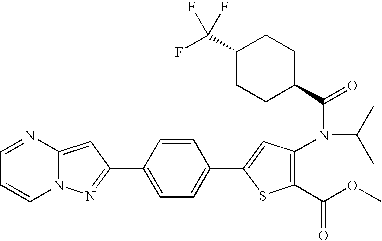

Intermediate 76 (150 mg), 2-[4-(4,4,5,5-tetramethyl-1,3,2-dioxaborolan-2-yl)phenyl]pyrazolo-[1,5-a]pyrimidine (115 mg, a synthesis of which is described above as Intermediate 10), sodium carbonate (95 mg) and tetrakis(triphenylphosphine)palladium (0) (35 mg) in 1,4-dioxane (1.5 mL) and water (0.5 mL) were heated in a microwave at 100° C. for 20 mins. The reaction was evaporated in vacuo and the residue was taken into DCM, washed with sodium bicarbonate solution, and was separated using a hydrophobic frit. The organics were evaporated in vacuo and the crude material was purified by ISCO Companion silica chromatography, eluting with a gradient 0-100% EtOAc in cyclohexane to give the title compound.

MS calcd for (C₂₉H₂₉F₃N₄O₃S+H)⁺: 571

MS found (electrospray): (M+H)⁺=571

### Alternative Preparation of Intermediate 59 (Method B)

### Methyl 3-((1-methylethyl){[trans-4-(trifluoromethyl)cyclohexyl]carbonyl}amino)-5-(4-pyrazolo[1,5-a]pyrimidin-2-ylphenyl)-2-thiophenecarboxylate

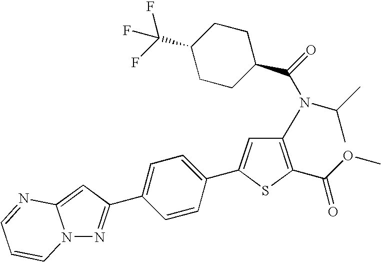

Tetrakis (triphenylphosphine) palladium (0) (24 mg) was added to a mixture of Intermediate 157 (200 mg), 2-(4-bromophenyl)pyrazolo[1,5-a]pyrimidine (synthesis of which is described as Intermediate 9) and cesium fluoride (195 mg) in DME (3 mL) and water (1 mL). This was heated to 100° C. under nitrogen for 2 h. On cooling, the reaction was poured into 8% sodium bicarbonate solution (20 mL) and extracted with DCM (2×10 mL). The combined organic extracts were washed with water (10 mL), dried (AQ extraction cartridge) and evaporated. The residue was dissolved in the minimum volume of DCM and purified by ISCO silica column chromatography eluting with EtOAc in cyclohexane (0-100%) to give the title compound.

MS calcd for (C₂₉H₂₉F₃N₄O₃S+H)⁺: 571

MS found (electrospray): (M+H)⁺=571

### Intermediate 60

### 2-(4-Bromophenyl)-4-nitro-1,3-benzoxazole

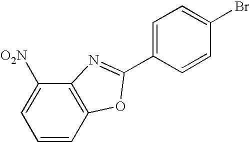

A mixture of 2-amino-3-nitrophenol (278 mg) and 4-bromobenzoyl chloride (439 mg) in dry 1,4-dioxane (2 mL) was stirred and heated at 210° C. in a microwave for 15 mins. The mixture was slowly added to a vigorously stirred 1 M sodium hydroxide solution (50 mL). The precipitate was collected by filtration, washed with water and dried in a vacuum oven to give the title compound.

MS calcd for (C₁₃H₇BrN₂O₃+H)⁺: 319/321

MS found (electrospray): (M+H)⁺=319/321

### Intermediate 61

### 2-(4-Bromophenyl)-1,3-benzoxazol-4-amine

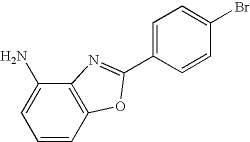

A stirred solution of 2-(4-bromophenyl)-4-nitro-1,3-benzoxazole (5.2 g, a synthesis of which is described above as Intermediate 60), in THF (250 mL) at 21° C. was treated with tin (II) chloride (7.1 g) and concentrated HCl (2 mL). The mixture was stirred for 4 h. Further portions of tin (II) chloride (7.1 g) and concentrated HCl (2 mL) were added and the mixture was stirred overnight. The reaction was evaporated in vacuo and the residue was treated with water (200 mL) and EtOAc (200 mL). The mixture was basified with 2N sodium hydroxide solution and the resulting emulsion was filtered through a celite pad which was washed well with EtOAc. The filtrate was separated and the aqueous extracted with EtOAc (×2). The combined organics were washed with brine, dried over sodium sulphate and evaporated in vacuo to give the title compound.

MS calcd for (C₁₃H₉BrN₂O+H)⁺: 289/291

MS found (electrospray): (M+H)⁺=289/291

### Intermediate 62

### 2-[4-(4,4,5,5-Tetramethyl-1,3,2-dioxaborolan-2-yl)phenyl]-1,3-benzoxazol-4-amine

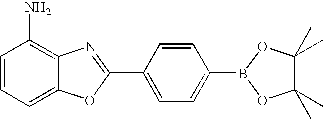

A mixture of Intermediate 61 (3.0 g), bis(pinacolato)diboron (3.99 g), potassium acetate (3.05 g) and PdCl₂(dppf) (558 mg) in dry 1,4-dioxane (60 mL) was stirred and heated at reflux for 2 h under nitrogen. The reaction was allowed to cool and was evaporated in vacuo. The residue was partitioned between DCM and water and the aqueous phase was separated and extracted with DCM (×2). The combined organics were washed with brine, dried with sodium sulphate and evaporated in vacuo. The crude material was purified by ISCO Companion silica chromatography eluting with a gradient 0-100% EtOAc in cyclohexane to give the title compound.

MS calcd for (C₁₉H₂₁BN₂O₃+H)⁺: 337

MS found (electrospray): (M+H)⁺=337

### Intermediate 63

### Methyl 5-[4-(4-amino-1,3-benzoxazol-2-yl)phenyl]-3-[[(trans-4-methylcyclohexyl)carbonyl](1-methylethyl)amino]-2-thiophenecarboxylate

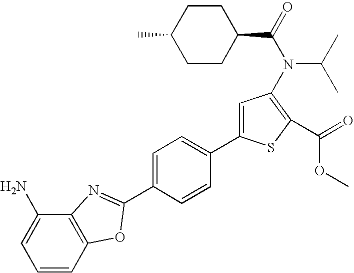

A mixture of Intermediate 62 (0.66 g), 5-iodo-3-[[(trans-4-methylcyclohexyl)carbonyl](1-methylethyl)amino]-2-thiophenecarboxylic acid (0.67 g, a synthesis of which is described above as Intermediate 5), tetrakis(triphenylphosphine)palladium (0) (245 mg), and 2M sodium carbonate solution (3.75 mL) in dry DMF (10 mL) was stirred and heated at 100° C. under nitrogen for 1.5 h. The reaction was allowed to cool and was evaporated in vacuo.

The residue was partitioned between EtOAc and water and the aqueous phase was separated and extracted with EtOAc (×2). The combined organics were washed with water and brine, dried over sodium sulphate and evaporated in vacuo. The crude material was purified by ISCO Companion silica chromatography eluting with a gradient 0-100% EtOAc in cyclohexane to give the title compound.

MS calcd for (C₃₀H₃₃N₃O₄S+H)⁺: 532

MS found (electrospray): (M+H)⁺=532

### Intermediate 64

### {4-[[(trans-4-Methylcyclohexyl)carbonyl](1-methylethyl)amino]-5-[(methyloxy)carbonyl]-2-thienyl}boronic acid

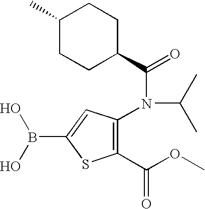

A solution of LDA (2.0M solution in THF/heptane/ethyl benzene, 46.4 mL) was stirred under nitrogen and cooled to −78° C. A solution of methyl 3-[[(trans-4-methylcyclohexyl)carbonyl](1-methylethyl)amino]-2-thiophenecarboxylate (10.0 g, a synthesis of which is described as Intermediate 21) in dry THF (100 mL) was added dropwise, keeping the temperature less than −70° C. The resulting mixture was stirred at −78° C. for 1 h, and was then treated dropwise with a solution of trimethyl borate (10.4 mL) in dry THF (50 mL) keeping the temperature below −70° C. The reaction was stirred for 30 mins and was then quenched by the dropwise addition of 2M HCl solution (150 mL). The reaction was allowed to warm to 21° C. and the phases were separated. The aqueous layer was extracted with EtOAc (×3), and the combined organics were washed with brine, dried over sodium sulphate and evaporated in vacuo. The residue was triturated with IPA/water (1:1) and was filtered and washed with a further portion of IPA/water (1:1). The material was then dried in a vacuum oven at 50° C. to give the title compound.

MS calcd for (C₁₇H₂₆BNO₅S+H)⁺: 368

MS found (electrospray): (M+H)⁺=368

### Intermediate 65

### 3-Amino-1,2-benzenediol

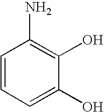

To a stirred solution 2,3-bis(methyloxy)aniline (3.0 g), in dry DCM (30 mL) at −45° C. under nitrogen was added a solution of boron tribromide (1M in DCM, 68.5 mL) dropwise over 10 mins. After 0.5 h at −45° C. the cooling bath was removed and the mixture was allowed to stand at room temperature overnight. The mixture was then cooled to 5° C. and water was added dropwise with stirring. The aqueous phase was adjusted to pH 5/6 using saturated sodium bicarbonate solution and the resulting mixture was extracted with DCM (×2). The aqueous layer was separated using a hydrophobic frit and was passed down a pre-conditioned OASIS HLB cartridge. The cartridge was eluted with methanol to give the title compound.

¹H NMR (d₆-DMSO) δ 6.35 (1H, t), 6.10 (1H, d), 6.06 (1H, d), amino and hydroxyl protons not seen.

### Intermediate 66

### 5-Iodopyrazolo[1,5-a]pyrimidine

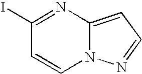

A mixture of 5-chloropyrazolo[1,5-a]pyrimidine (2.05 g), and sodium iodide (10 g) in dry acetonitrile (90 mL) was added acetyl chloride (4.78 mL). The resultant mixture was heated at reflux for 20 h. The resultant brown suspension was cooled to room temperature and was then poured onto a stirring mixture of 0.5M sodium carbonate (400 mL), 1.0M aqueous sodium sulphate (100 mL) and Et₂O (700 mL). The organic layer was separated, dried over magnesium sulphate and evaporated in vacuo. The crude material was purified using Biotage silica chromatography, eluting with DCM to give the title compound.

MS calcd for (C₆H₄IN₃+H)⁺: 246

MS found (electrospray): (M+H)⁺=246

### Intermediate 67

### Methyl 3-[[(trans-4-methylcyclohexyl)carbonyl](1-methylethyl)amino]-5-(4-pyrazolo[1,5-a]pyrimidin-5-ylphenyl)-2-thiophenecarboxylate

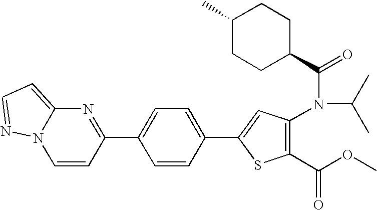

A mixture of tetrakis(triphenylphosphine)palladium (0) (73 mg), methyl 5-iodo-3-[[(trans-4-methylcyclohexyl)carbonyl](1-methylethyl)amino]-2-thiophenecarboxylate (1.58 g, a synthesis of which is described as Intermediate 5), and benzene-1,4-diyldiboronic acid (2.22 g) in 1,4-dioxane (25 mL) and 2N sodium carbonate (7 mL) was heated at 100° C. for 20 h. The reaction was evaporated in vacuo and the residue was partitioned between water and DCM. The aqueous phase was separated and was extracted with DCM (×2). The organic phases were combined and evaporated in vacuo. The crude material was purified by ISCO Companion silica chromatography, eluting with a gradient 0-100% EtOAc in cyclohexane to give (4-{4-[[(trans-4-methylcyclohexyl)carbonyl](1-methylethyl)amino]-5-[(methyloxy)carbonyl]2-thienyl}phenyl)boronic acid (as a 1:1 mixture with the corresponding anhydride dimer). Tetrakis(triphenylphosphine)palladium (0) (24 mg) was added to a stirred mixture of Intermediate 66 (71 mg) and (4-{4-[[(trans-4-methylcyclohexyl)carbonyl](1-methylethyl)amino]-5-[(methyloxy)carbonyl]-2-thienyl}phenyl)boronic acid (130 mg, prepared above) in 1,4-dioxane (3 mL) and 2N sodium carbonate solution (0.5 mL). The mixture was heated at 100° C. in a sealed vessel for 3 h. The reaction was evaporated in vacuo and the residue was partitioned between DCM and water. The organics were separated using a hydrophobic frit and were evaporated in vacuo. The crude product was purified by ISCO Companion silica chromatography eluting with a gradient 10-50% EtOAc in cyclohexane to give the title compound.

MS calcd for (C₂₉H₃₂N₄O₃S+H)⁺: 517

MS found (electrospray): (M+H)⁺=517

### Intermediate 68

### 3-Iodo-1,2-benzenediol

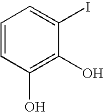

To a stirred solution of 1-iodo-2,3-bis(methyloxy)benzene (1.01 g) in dry DCM (6 mL) under nitrogen at −45° C. was added a solution of boron tribromide (1 M in DCM, 4.24 mL). After 0.5 h at −45° C. the cooling bath was removed and the mixture was allowed to stir at room temperature for 1 h. The mixture was then cooled to −9° C. and water was added dropwise with stirring. The organics were separated using a hydrophobic frit and the aqueous phase was extracted with DCM. The combined organics were evaporated in vacuo to give the title compound.

MS calcd for (C₆H₅IO₂—H)⁻: 235

MS found (electrospray): (M−H)⁻=235

### Intermediate 69

### 3-[(4-Bromophenyl)ethynyl]-1,2-benzenediol

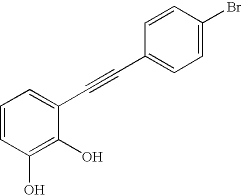

Dichloropalladium bistriphenylphosphine (0.14 g) and copper (I) iodide (0.036 g) were added to a degassed solution of Intermediate 68 (0.89 g), bromophenyl acetylene (1.02 g) and triethylamine (2.11 mL) in THF (12 mL). The mixture was heated at 50° C. under nitrogen for 2.5 h. The reaction was allowed to cool and was evaporated in vacuo. The residue was partitioned between DCM and water. The organics were separated using a hydrophobic frit and were evaporated in vacuo. The crude material was purified by ISCO Companion silica chromatography, eluting with a gradient 0-70% EtOAc in cyclohexane to give the title compound.

¹H NMR (d₆-DMSO) δ 9.57 (1H, s), 9.07 (1H, s), 7.61 (2H, d), 7.46 (2H, d), 6.83 (2H, dt), 6.04 (1H, t).

### Intermediate 70

### 2-(4-Bromophenyl)-1-benzofuran-7-ol

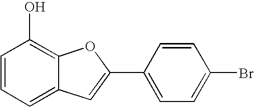

A stirred mixture of Intermediate 69 (380 mg) in acetonitrile (20 mL) and saturated aqueous sodium bicarbonate (2 mL) was heated at 70° C. for 5.5 h. The reaction was allowed to cool and was evaporated in vacuo. The residue was partitioned between DCM and water (some solid remained). The organics were separated using a hydrophobic frit and were evaporated in vacuo. The crude material was purified by ISCO Companion silica chromatography, eluting with a gradient 0-45% EtOAc in cyclohexane to give the title compound.

¹H NMR (CDCl₃) δ 7.73 (2H, dt), 7.58 (2H, dt), 7.19-7.10 (2H, m), 7.04 (1H, s), 6.86 (1H, dd), hydroxyl proton not seen.

### Intermediate 71

### Methyl 5-[4-(7-hydroxy-1-benzofuran-2-yl)phenyl]-3-[[(trans-4-methylcyclohexyl)carbonyl](1-methylethyl)amino]-2-thiophenecarboxylate

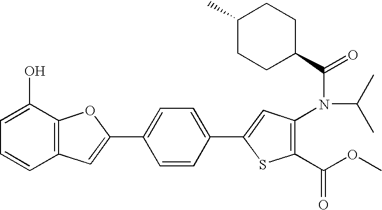

A mixture of Intermediate 70 (45 mg), Intermediate 64 (85 mg), cesium fluoride (240 mg) and tetrakis(triphenylphosphine)palladium (0) (18 mg) in DME/water (1:1, 4 mL) was heated to 100° C. for 3.5 h and was then allowed to cool to room temperature. A further portion of Intermediate 64 (40 mg) was added and the mixture was heated to 100° C. for 2 h and was then allowed to cool to room temperature. The reaction was evaporated in vacuo and the residue was partitioned between DCM and water. The organic phase was separated using a hydrophobic frit and was evaporated in vacuo. The crude material was purified by ISCO Companion silica chromatography, eluting with a gradient 0-60% EtOAc in cyclohexane to give the title compound.

MS calcd for (C₃₁H₃₃NO₅S+H)⁺: 532

MS found (electrospray): (M+H)⁺=532

### Intermediate 72

### 2-(4-Bromophenyl)-1,3-benzoxazol-7-ol

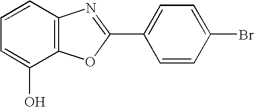

A mixture of Intermediate 65 (1.26 g), 4-bromobenzoyl chloride (1.76 g) and polyphosphoric acid (46 g) was heated at 180° C. with stirring for 5 h. The reaction was allowed to cool to room temperature and was partitioned between DCM and water. The aqueous phase was separated and extracted with DCM. The combined organics were evaporated in vacuo. The crude material was purified by ISCO Companion silica chromatography, eluting with a gradient 0-20% EtOAc in cyclohexane to give the title compound.

MS calcd for (C₁₃H₈BrNO₂+H)⁺: 290/292

MS found (electrospray): (M+H)⁺=290/292

### Intermediate 73

### Methyl 5-[4-(7-hydroxy-1,3-benzoxazol-2-yl)phenyl]-3-[[(trans-4-methylcyclohexyl)carbonyl](1-methylethyl)amino]-2-thiophenecarboxylate

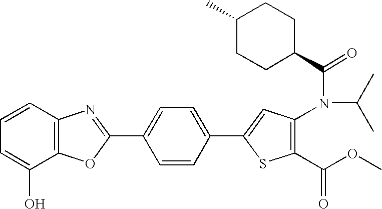

A mixture of Intermediate 72 (41 mg), {4-[[(trans-4-Methylcyclohexyl)carbonyl](1-methylethyl)amino]-5-[(methyloxy)carbonyl]-2-thienyl}boronic acid (85 mg, a synthesis of which is described above as Intermediate 64), cesium fluoride (240 mg) and tetrakis(triphenylphosphine)palladium (0) (18 mg) in DME/water (1:1, 4 mL) was heated at 100° C. with rapid stirring for 2.5 h. The reaction mixture was concentrated in vacuo to approximately half volume and was partitioned between 0.5M HCl and DCM. The organics were separated using a hydrophobic frit and were evaporated in vacuo. The crude material was purified by ISCO Companion silica chromatography, eluting with a gradient 0-40% EtOAc in cyclohexane to give the title compound.

MS calcd for (C₃₀H₃₂N₂O₅S+H)⁺: 533

MS found (electrospray): (M+H)⁺=533

### Intermediate 74

### Methyl 5-(4-imidazo[1,2-a]pyridin-2-ylphenyl)-3-((1-methylethyl){[trans-4-(trifluoromethyl)cyclohexyl]carbonyl}amino)-2-thiophenecarboxylate

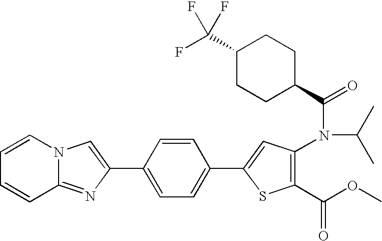

Intermediate 55 (200 mg) was dissolved in DCM (6 mL). Triphenylphosphine (334 mg) and Intermediate 57 (200 mg) were added and the reaction was heated at 45° C. for 2 days. Sodium bicarbonate solution was added to the reaction and the mixture was stirred at room temperature for 1 h. Methanol (0.5 mL) was added (to improve solubility) and the organics were separated using a hydrophobic frit and the solvent was evaporated in vacuo. The crude material was purified by ISCO Companion silica chromatography eluting with a gradient 5-100% EtOAc in cyclohexane to give the title compound.

MS calcd for (C₃₀H₃₀F₃N₃O₃S+H)⁺: 570

MS found (electrospray): (M+H)⁺=570

### Intermediate 75

### Methyl 3-((1-methylethyl){[trans-4-(trifluoromethyl)cyclohexyl]carbonyl}amino)-2-thiophenecarboxylate

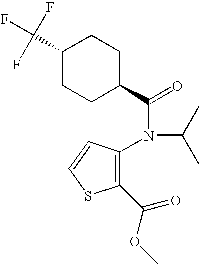

A mixture of methyl 3-[(1-methylethyl)amino]-2-thiophenecarboxylate (1.257 g, a synthesis of which is described above as Intermediate 20), trans-4-(trifluoromethyl)cyclohexanecarbonyl chloride (2.03 g, a synthesis of which is described above as Intermediate 57) and triphenylphosphine (3.31 g) in DCM (6 mL) was stirred at 50° C. under nitrogen overnight. A further portion of trans-4-(trifluoromethyl)cyclohexanecarbonyl chloride (1.352 g, a synthesis of which is described above as Intermediate 57) was added and the mixture was stirred at 50° C. under nitrogen overnight. Crystals precipitated out and were collected by filtration. The filtrate was partitioned between DCM and water. The aqueous layer was separated and extracted with DCM. The organics were then washed with water. The combined organics were evaporated and were purified by ISCO Companion silica chromatography, eluting with a gradient 0-60% EtOAc in cyclohexane. Product containing fractions were evaporated and combined with the crystals obtained earlier to give the title compound.

MS calcd for (C₁₇H₂₂F₃NO₃S+H)⁺: 378

MS found (electrospray): (M+H)⁺=378

### Intermediate 76

### Methyl 5-iodo-3-((1-methylethyl){[trans-4-(trifluoromethyl)cyclohexyl]carbonyl}amino)-2-thiophenecarboxylate

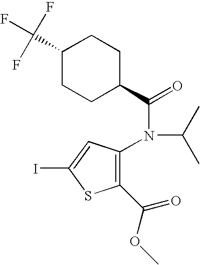

A solution of LDA (2.0M solution in THF/heptane/ethyl benzene, 10 mL) was cooled to −79° C. under an atmosphere of nitrogen. Intermediate 75 (2.5 g) was dissolved in dry THF (20 mL) and was added slowly over 10 mins, keeping the internal temperature of the reaction in the range −80° C. to −70° C. The dropping funnel was rinsed with dry THF (3 mL). The reaction was stirred at −75° C. for 1.75 h. A solution of iodine (3.368 g) in dry THF (15 mL) was then added dropwise over 10 mins, keeping the internal temperature less than −70° C. The dropping funnel was rinsed with dry THF (4 mL). After 45 mins the reaction was quenched with saturated ammonium chloride solution (14 mL). The reaction was allowed to warm to 0° C. and was then washed with 5% sodium thiosulphate solution (60 mL). The organic phase was separated and evaporated. The residue was partitioned between DCM and water. The aqueous layer was extracted with DCM and the combined organics were washed with water. The organics were evaporated in vacuo and were purified by ISCO Companion silica chromatography, eluting with a gradient 0-100% EtOAc in cyclohexane to give the title compound.

MS calcd for (C₁₇H₂₁F₃INO₃S+H)⁺: 504

MS found (electrospray): (M+H)⁺=504

### Intermediate 77

### Methyl 5-(4-imidazo[2,1-b][1,3]thiazol-6-ylphenyl)-3-((1-methylethyl){[trans-4-(trifluoromethyl)cyclohexyl]carbonyl}amino)-2-thiophenecarboxylate

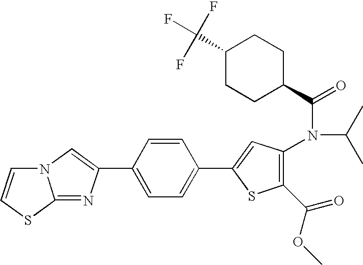

A mixture of Intermediate 76 (100 mg), 6-[4-(4,4,5,5-tetramethyl-1,3,2-dioxaborolan-2-yl)phenyl]imidazo[2,1-b][1,3]thiazole (65 mg, a synthesis of which is described above as Intermediate 24), sodium carbonate (63 mg), and tetrakis(triphenylphosphine)palladium (0) (23 mg) in water (0.5 mL) and 1,4-dioxane (1.5 mL) were heated in a microwave at 100° C. for 25 mins. The reaction was evaporated in vacuo and the residue was partitioned between DCM and water. The organics were separated and evaporated in vacuo. The residue was purified by ISCO Companion silica chromatography, eluting with 0-100% EtOAc in cyclohexane to give the title compound.

MS calcd for (C₂₈H₂₈F₃N₃O₃S₂+H)⁺: 576

MS found (electrospray): (M+H)⁺=576

### Intermediate 78

### Methyl 5-(4-furo[3,2-b]pyridin-2-ylphenyl)-3-((1-methylethyl){[trans-4-(trifluoromethyl)cyclohexyl]carbonyl}amino)-2-thiophenecarboxylate

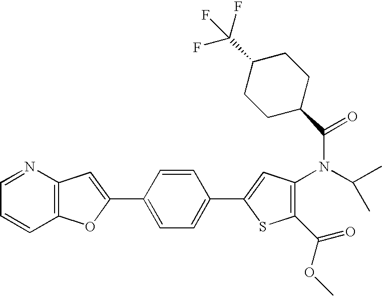

A mixture of Intermediate 76 (150 mg), 2-[4-(4,4,5,5-tetramethyl-1,3,2-dioxaborolan-2-yl)phenyl]furo[3,2-b]pyridine (96 mg, a synthesis of which is described above as Intermediate 52), sodium carbonate (95 mg), and tetrakis(triphenylphosphine)palladium (0) (34 mg) in water (0.5 mL) and 1,4-dioxane (1.5 mL) were heated in a microwave at 100° C. for 25 mins. The reaction was evaporated in vacuo and the residue was partitioned between DCM and water. The organics were separated and evaporated in vacuo. The residue was purified by ISCO Companion silica chromatography, eluting with 0-100% EtOAc in cyclohexane to give the title compound.

MS calcd for (C₃₀H₂₉F₃N₂O₄S+H)⁺: 571

MS found (electrospray): (M+H)⁺=571

### Intermediate 79

### 2-(4-Bromophenyl)-1,3-benzoxazol-6-amine

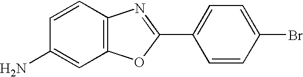

A solution of Intermediate 56 (2.503 g) in THF (125 mL) was stirred at room temperature and was treated with tin (II) chloride (3.42 g) and concentrated HCl (1 mL). The mixture was stirred at room temperature for 5 h. A further portion of tin (i) chloride (3.42 g) and concentrated HCl (1 mL) were added and the reaction was stirred at room temperature for 18 h. A further portion of tin (II) chloride (800 mg) was added and the reaction was stirred at room temperature for 3 days. The reaction was evaporated in vacuo and the residue was treated with 2M sodium hydroxide solution and EtOAc. The resulting emulsion was filtered and washed with more EtOAc. The filtrate was separated and the aqueous layer was extracted with EtOAc (×2). The combined organics were dried over magnesium sulphate and evaporated in vacuo to give the title compound.

MS calcd for (C₁₃H₉BrN₂O+H)⁺: 289/291

MS found (electrospray): (M+H)⁺=289/291

### Intermediate 80

### 2-[4-(4,4,5,5-Tetramethyl-1,3,2-dioxaborolan-2-yl)phenyl]-1,3-benzoxazol-6-amine

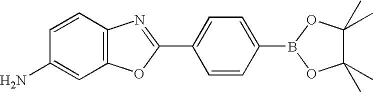

Intermediate 79 (0.5 g), bis(pinacolato)diboron (0.66 g), potassium acetate (0.51 g) and PdCl₂(dppf) (93 mg) in dry 1,4-dioxane (10 mL) were stirred and heated at 100° C. for 30 mins. The reaction was evaporated in vacuo and the residue was partitioned between DCM and water. The aqueous phase was separated and extracted with DCM (×3). The combined organics were washed with brine, dried over magnesium sulphate and were evaporated in vacuo. The crude material was purified using ISCO Companion silica chromatography, eluting with a gradient 0-100% EtOAc in cyclohexane to give the title compound.

MS calcd for (C₁₉H₂₁BN₂O₃+H)⁺: 337

MS found (electrospray): (M+H)⁺=337

### Intermediate 81

### Methyl 5-[4-(6-amino-1,3-benzoxazol-2-yl)phenyl]-3-[[(trans-4-methylcyclohexyl)carbonyl](1-methylethyl)amino]-2-thiophenecarboxylate

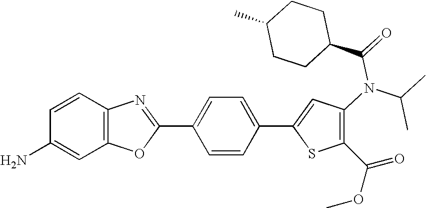

Intermediate 80 (372 mg), methyl 5-iodo-3-[[(trans-4-methylcyclohexyl)carbonyl](1-methylethyl)amino]-2-thiophenecarboxylate (361 mg, a synthesis of which is described above as Intermediate 5B), 2N sodium carbonate solution (1.66 mL) and tetrakis(triphenylphosphine)palladium (0) (134 mg) were dissolved in dry DMF (6.5 mL) and were heated at 100° C. under nitrogen for 1 h. The reaction was evaporated in vacuo and the residue was partitioned between EtOAc and water. The aqueous was extracted with EtOAc (×3). The combined organics were washed with brine, dried over magnesium sulphate and were evaporated in vacuo. The crude material was purified by ISCO Companion silica chromatography, eluting with a gradient 0-100% EtOAc in cyclohexane to give the title compound.

MS calcd for (C₃₀H₃₃N₃O₄S+H)⁺: 532

MS found (electrospray): (M+H)⁺=532

### Intermediate 82

### Methyl 3-(4-bromophenyl)-2-propynoate

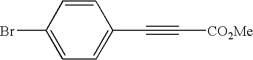

A solution of 3-(4-bromophenyl)-2-propynoic acid (100 mg) in 1.25M HCl in methanol solution (10 mL) was allowed to stand at room temperature for 4 days and was evaporated in vacuo to give the title compound.

MS calcd for (C₁₀H₇BrO₂+H)⁺: 239/241

MS found (electrospray): (M+H)⁺=239/241

### Intermediate 83

### 2-(4-Bromophenyl)pyrazolo[1,5-b]pyridazine

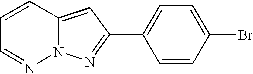

1-Aminopyridazinium iodide (100 mg) and Intermediate 82 (110 mg) in acetonitrile (2.5 mL) under nitrogen were treated dropwise with DBU (0.13 mL) over 5 mins. The mixture was stirred for 2 h and was evaporated in vacuo. The residue was partitioned between 1N HCl and EtOAc. The organic phase was separated, dried over sodium sulphate and evaporated in vacuo to give a gum. The gum was dissolved in ethanol (3 mL) and was treated with 2N sodium hydroxide solution (3 mL) and the mixture was heated at reflux for 19 h. The reaction mixture was evaporated in vacuo and the residue was partitioned between EtOAc and 2N HCl. The aqueous phase was separated and was extracted with EtOAc (×3). The combined organics were dried over sodium sulphate and were evaporated in vacuo to give a solid. The solid was combined with copper powder (100 mg) and quinoline (4 mL) and was heated at 180° C. under nitrogen for 3 h. The reaction was allowed to cool and was partitioned between 2N HCl and EtOAc. The organics were separated, washed with 2N HCl (×3), dried over sodium sulphate and evaporated in vacuo. The crude material was purified by ISCO Companion silica chromatography, eluting with a gradient 5-65% EtOAc in cyclohexane to give the title compound.

MS calcd for (C₁₂H₈BrN₃+H)⁺: 274/276

MS found (electrospray): (M+H)⁺=274/276

### Intermediate 84

### Methyl 3-[[(trans-4-methylcyclohexyl)carbonyl](1-methylethyl)amino]-5-(4-pyrazolo[1,5-b]pyridazin-2-ylphenyl)-2-thiophenecarboxylate

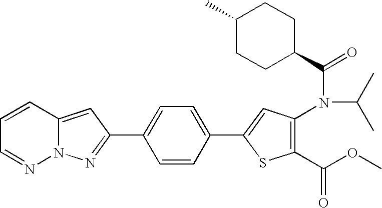

A mixture of Intermediate 83 (42 mg), {4-[[(trans-4-methylcyclohexyl)carbonyl](1-methylethyl)amino]-5-[(methyloxy)carbonyl]-2-thienyl}boronic acid (85 mg, a synthesis of which is described above as Intermediate 64), cesium fluoride (240 mg) and tetrakis(triphenylphosphine)palladium (0) (18 mg) in DME/water (1:1, 4 mL) was heated at 100° C. with rapid stirring for 3 h. The reaction mixture was concentrated in vacuo to approximately half volume and was partitioned between water and DCM. The organics were separated using a hydrophobic frit and were evaporated in vacuo. The crude material was purified by ISCO Companion silica chromatography, eluting with a gradient 10-50% EtOAc in cyclohexane to give the title compound.

MS calcd for (C₂₉H₃₂N₄O₃S+H)⁺: 517

MS found (electrospray): (M+H)⁺=517

### Intermediate 85

### Methyl 3-((1-methylethyl){[trans-4-(trifluoromethyl)cyclohexyl]carbonyl}amino)-5-(4-[1,3]oxazolo[4,5-b]pyridin-2-ylphenyl)-2-thiophenecarboxylate

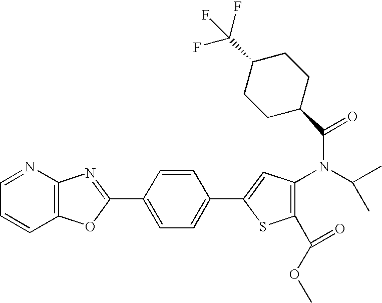

A mixture of Intermediate 76 (150 mg), 2-[4-(4,4,5,5-tetramethyl-1,3,2-dioxaborolan-2-yl)phenyl][1,3]oxazolo[4,5-b]pyridine (96 mg, a synthesis of which is described above as Intermediate 37), sodium carbonate (95 mg), tetrakis(triphenylphosphine)palladium (0) (34 mg) in water (0.5 mL) and 1,4-dioxane (1.5 mL) was heated in a microwave at 100° C. for 25 mins. A further portion of 2-[4-(4,4,5,5-tetramethyl-1,3,2-dioxaborolan-2-yl)phenyl][1,3]oxazolo[4,5-b]pyridine (29 mg, a synthesis of which is described above as Intermediate 37) was added and the mixture was heated in a microwave at 100° C. for 20 mins. The mixture was evaporated in vacuo and the residue was partitioned between DCM and water. The aqueous layer was extracted with DCM and the combined organics were evaporated in vacuo and purified by ISCO Companion silica chromatography, eluting with a gradient 0-100% EtOAc in cyclohexane to give the title compound.

MS calcd for (C₂₉H₂₈F₃N₃O₄S+H)⁺: 572

MS found (electrospray): (M+H)⁺=572

### Intermediate 86

### Methyl 3-((1-methylethyl){[trans-4-(trifluoromethyl)cyclohexyl]carbonyl}amino)-5-[4-(5-methylpyrazolo[1,5-a]pyrimidin-2-yl)phenyl]-2-thiophenecarboxylate

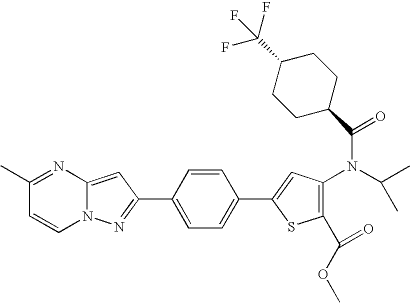

Intermediate 30 (116 mg, prepared by method B), Intermediate 76 (175 mg), sodium carbonate (110 mg) and tetrakis(triphenylphosphine)palladium (0) (40 mg) were dissolved in water (0.5 mL) and 1,4-dioxane (1.5 mL) and were heated in a microwave at 100° C. for 30 mins. The mixture was evaporated in vacuo and was partitioned between DCM and water. The organics were washed with water and the aqueous layer was extracted with DCM. The combined organics were evaporated in vacuo and were purified by ISCO Companion silica chromatography, eluting with a gradient 0-100% EtOAc in cyclohexane to give the title compound.

MS calcd for (C₃₀H₃₁F₃N₄O₃S+H)⁺: 585

MS found (electrospray): (M+H)⁺=585

### Intermediate 87

### 2-(4-Bromophenyl)-6-nitroimidazo[1,2-a]pyridine

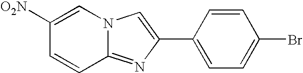

A mixture of 5-nitro-2-pyridinamine (270 mg) and 2-bromo-1-(4-bromophenyl)ethanone (540 mg) in acetone (2.5 mL) was stirred and heated in a microwave at 100° C. for 15 mins, 130° C. for 20 mins, then 120° C. for 10 mins. A precipitate formed which was filtered off and washed with acetone. The solid was dried in a vacuum oven to give the title compound.

MS calcd for (C₁₃H₈BrN₃O₂+H)⁺: 318/320

MS found (electrospray): (M+H)⁺=318/320

### Intermediate 88

### 2-(4-Bromophenyl)imidazo[1,2-a]pyridin-6-amine

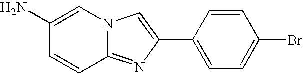

A solution of 2-(4-bromophenyl)-6-nitroimidazo[1,2-a]pyridine (2.61 g, a synthesis of which is described above as Intermediate 87) in THF (125 mL) was stirred at room temperature and was treated with tin (II) chloride (3.6 g) and concentrated HCl (1 mL). The mixture was stirred at room temperature for 2 h. The solvent was evaporated in vacuo and the residue was treated with 2N NaOH solution and chloroform. The resulting emulsion was filtered and the solid was washed with chloroform (×3). The filtrate was separated and the aqueous washed with chloroform (×1). The aqueous was extracted into EtOAc (×3). The combined organics were dried over magnesium sulphate and were evaporated in vacuo. This material was dissolved in THF (40 mL), stirred at room temperature and was treated with tin (II) chloride (1.18 g) and conc. HCl (0.5 mL). The mixture was stirred at room temperature for 18 h. The solvent was evaporated in vacuo and the residue was treated with 2N NaOH solution and EtOAc. The organic layer separated and was filtered through a pad of celite. The filtrate was evaporated in vacuo to give the title compound.

MS calcd for (C₁₃H₁₀BrN₃+H)⁺: 288/290

MS found (electrospray): (M+H)⁺=288/290

### Intermediate 89

### 2-[4-(4,4,5,5-Tetramethyl-1,3,2-dioxaborolan-2-yl)phenyl]imidazo[1,2-a]pyridin-6-amine

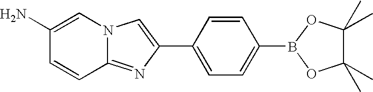

2-(4-Bromophenyl)imidazo[1,2-a]pyridin-6-amine (0.486 g, a synthesis of which is described above as Intermediate 88), bis(pinacolato)diboron (0.65 g), potassium acetate (0.49 g) and PdCl₂(dppf) (91 mg) in dry 1,4-dioxane (10 mL) were stirred and heated at 100° C. for 3 h. The solvent was evaporated in vacuo and the residue partitioned between DCM and water. This gave an emulsion so chloroform was added and the layers were separated. The aqueous was extracted with chloroform (×3). The combined organics were dried over magnesium sulphate and were evaporated in vacuo. The crude material was purified by silica SPE cartridge, eluting with 2% MeOH in DCM, 20% MeOH in DCM, 50% MeOH in DCM and 100% MeOH to give the title compound.

MS calcd for (C₁₉H₂₂BN₃O₂+H)⁺: 336

MS found (electrospray): (M+H)⁺=336

### Intermediate 90

### Methyl 5-[4-(6-aminoimidazo[1,2-a]pyridin-2-yl)phenyl]-3-[[(trans-4-methylcyclohexyl)carbonyl](1-methylethyl)amino]-2-thiophenecarboxylate

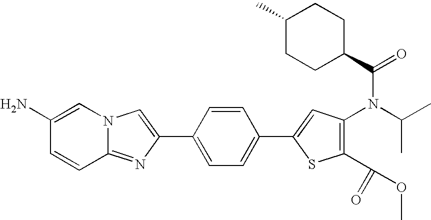

Intermediate 89 (328 mg), methyl 5-iodo-3-[[(trans-4-methylcyclohexyl)carbonyl](1-methylethyl)amino]-2-thiophenecarboxylate (333 mg, a synthesis of which is described above as Intermediate 5), 2N sodium carbonate solution (1.5 mL) and tetrakis(triphenylphosphine)palladium (0) (119 mg) were dissolved in dry DMF (6.5 mL) and were heated at 100° C. under nitrogen for 1.5 h. The reaction was evaporated in vacuo and the residue was partitioned between EtOAc and water. The aqueous was extracted with EtOAc (×3) and chloroform (×2). The organics were combined, dried over magnesium sulphate and evaporated in vacuo. The crude material was purified by ISCO Companion silica chromatography eluting with a gradient 0-100% EtOAc in cyclohexane, followed by 0-100% MeOH in DCM. This gave impure material which was re-purified by silica SPE cartridge, eluting with stepped solvents; 2% MeOH in DCM to 20% MeOH in DCM to give the title compound.

MS calcd for (C₃₀H₃₄N₄O₃S+H)⁺: 531

MS found (electrospray): (M+H)⁺=531

### Intermediate 91

### 1,1-Dimethylethyl 5-bromo-1H-benzimidazole-1-carboxylate

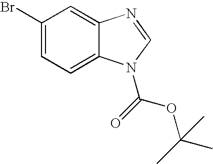

To a suspension of 5-bromo-1H-benzimidazole (830 mg), in DCM (100 mL) was added triethylamine (0.70 mL), DMAP (62 mg) and di-tert-butyl dicarbonate (1.11 g). The mixture was allowed to stir at room temperature for 20 h. The reaction mixture was washed with water, separated using a hydrophobic frit and was evaporated in vacuo. The crude material was purified by ISCO Companion silica chromatography, eluting with a gradient 15-50% EtOAc in cyclohexane to give the title compound.

¹H NMR (CDCl₃) δ 8.37 (1H, s), 7.86 (1H, d), 7.80 (1H, d), 7.42 (1H, dd), 1.66 (9H, s).

### Intermediate 92

### 1,1-Dimethylethyl 6-(4-{4-[[(trans-4-methylcyclohexyl)carbonyl](1-methylethyl)amino]-5-[(methyloxy)carbonyl]-2-thienyl}phenyl)-1H-benzimidazole-1-carboxylate

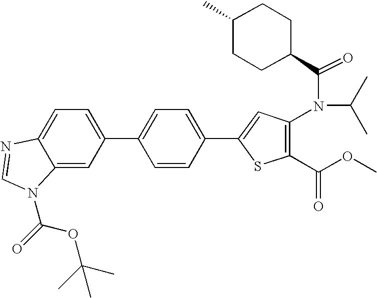

A mixture of tetrakis(triphenylphosphine)palladium (0) (73 mg), methyl 5-iodo-3-[[(trans-4-methylcyclohexyl)carbonyl](1-methylethyl)amino]-2-thiophenecarboxylate (1.58 g, a synthesis of which is described as Intermediate 5, method B), and benzene-1,4-diyldiboronic acid (2.22 g) in 1,4-dioxane (25 mL) and 2N sodium carbonate (7 mL) was heated at 100° C. for 20 h. The reaction was evaporated in vacuo and the residue was partitioned between water and DCM. The aqueous phase was separated and was extracted with DCM (×2). The organic phases were combined and evaporated in vacuo. The crude material was purified by ISCO Companion silica chromatography, eluting with a gradient 0-100% EtOAc in cyclohexane to give (4-{4-[[(trans-4-methylcyclohexyl)carbonyl](1-methylethyl)amino]-5-[(methyloxy)carbonyl]-2-thienyl}phenyl)boronic acid (as a 1:1 mixture with the corresponding anhydride dimer).

Tetrakis(triphenylphosphine)palladium (0) (26 mg) was added to a stirred mixture of Intermediate 91 (110 mg) and (4-{4-[[(trans-4-methylcyclohexyl)carbonyl](1-methylethyl)amino]-5-[(methyloxy)carbonyl]-2-thienyl}phenyl)boronic acid (160 mg, prepared above) in 1,4-dioxane (10 mL) and 2N sodium carbonate solution (2 mL). The mixture was heated at 80° C. for 4 h. The reaction was cooled, evaporated in vacuo and the residue was partitioned between DCM and water. The organics were separated using a hydrophobic frit and were evaporated in vacuo. The crude material was purified by ISCO Companion silica chromatography, eluting with a gradient 18-42% EtOAc in cyclohexane to give the title compound.

MS calcd for (C₃₅H₄₁N₃O₅S+H)⁺: 616

MS found (electrospray): (M+H)⁺=616

### Intermediate 93

### 1-(4-Bromophenyl)ethanone O-(4-nitrophenyl)oxime (˜6:1 mixture of E and Z isomers)

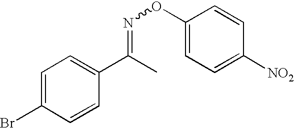

A solution of Intermediate 58 (2.14 g) in THF (10 mL) was stirred at 0° C. under nitrogen. 1.0M potassium tert-butoxide in THF (10 mL) was added and the resulting mixture was stirred under nitrogen with ice-bath cooling for 1.5 h. A solution of 1-fluoro-4-nitrobenzene (1.41 g) in THF (5 mL) was added and the mixture was stirred at 50° C. for 4 h. The mixture was cooled to room temperature and diluted to 250 mL with water. The resulting mixture was extracted with EtOAc (2×250 mL). The organic extracts were combined, washed with brine and dried over magnesium sulphate. The solvent was removed in vacuo to give title compound.

¹H NMR (CDCl₃) (major isomer) δ 8.29 (d, 2H), 7.83 (d, 2H), 7.72 (d, 2H), 7.53 (d, 2H), 3.34 (s, 3H).

### Intermediate 94

### 2-(4-Bromophenyl)-5-nitro-1-benzofuran

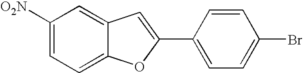

A mixture of Intermediate 93 (2.8 g), formic acid (100 mL) and concentrated HCl (15 mL) was stirred and heated at 80° C. for overnight. The mixture was cooled to room temperature and the solid collected by filtration, washed with water and dried under vacuum at 40° C. overnight to give the title compound.

¹H NMR (d₆-DMSO) δ 8.64 (d, 1H), 8.24 (dd, 1H), 7.93 (d, 2H), 7.89 (d, 1H), 7.76 (d, 2H), 7.72 (s, 1H)

### Intermediate 95

### 2-(4-Bromophenyl)-1-benzofuran-5-amine

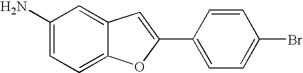

To a solution of Intermediate 94 (1.0 g) in THF (50 mL) was added tin (II) chloride (4.13 g) and concentrated HCl (20 drops). The mixture was stirred at room temperature overnight. A further quantity of tin (II) chloride (4.13 g) was added and stirring at room temperature was continued for 24 h. The mixture was diluted to 500 mL with water, basified with 2M sodium hydroxide solution, and extracted with EtOAc (2×400 mL). The biphasic mixture was filtered through a pad of Celite to remove insoluble tin residues. The organic layer was collected, washed with saturated brine (150 mL), dried over magnesium sulphate and evaporated in vacuo to give the title compound.

MS calcd for (C₁₄H₁₀BrNO+H)⁺: 288/290

MS found (electrospray): (M+H)⁺=288/290

### Intermediate 96

### 2-[4-(4,4,5,5-Tetramethyl-1,3,2-dioxaborolan-2-yl)phenyl]-1-benzofuran-5-amine

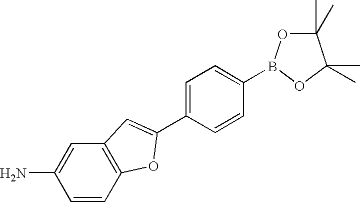

A solution of Intermediate 95 (470 mg) in 1,4-dioxane (15 mL) was treated with bis(pinacolato)diboron (620 mg), potassium acetate (482 mg) and PdCl₂(dppf) (80 mg). The mixture was heated at 100° C. under nitrogen for 4 h. The solvent was removed in vacuo and the residue was partitioned between water (50 mL) and DCM (50 mL). The organics were separated by hydrophobic frit and were evaporated in vacuo. The crude material was purified by ISCO Companion silica chromatography, eluting with a gradient 0-100% EtOAc in cyclohexane to give the title compound.

MS calcd for (C₂₀H₂₂BNO₃+H)⁺: 336

MS found (electrospray): (M+H)⁺=336

### Intermediate 97

### Methyl 5-[4-(5-amino-1-benzofuran-2-yl)phenyl]-3-[[(trans-4-methylcyclohexyl)carbonyl](1-methylethyl)amino]-2-thiophenecarboxylate

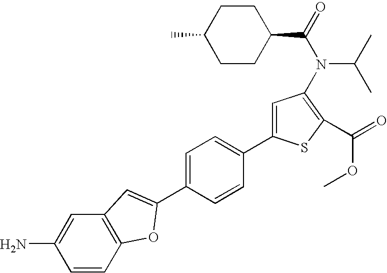

A mixture of Intermediate 96 (150 mg), Intermediate 5 (125 mg, prepared by method B), tetrakis(triphenylphosphine)palladium (0) (45 mg) and 2M sodium carbonate solution (1 mL) in DMF (2 mL) was stirred under nitrogen at 100° C. for 2 h. The mixture was allowed to cool and was evaporated in vacuo. The residue was partitioned between water (100 mL) and EtOAc (100 mL). The organics were separated, dried over magnesium sulphate and evaporated in vacuo. The crude material was purified by ISCO Companion silica chromatography, eluting with a gradient 0-100% EtOAc in cyclohexane to give the title compound.

MS calcd for (C₃₁H₃₄N₂O₄S+H)⁺: 531

MS found (electrospray): (M+H)⁺=531

### Intermediate 98

### Ethyl (1E)-N-[(3-nitrophenyl)oxy]ethanimidoate

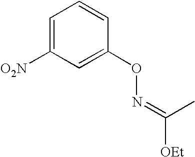

A solution of ethyl acetohydroximate (12.8 g) in THF (75 mL) was stirred under nitrogen at 0° C. 1M Potassium tert-butoxide in THF (136.5 mL) was added over 30 mins and the resulting mixture was stirred below −10° C. for 1.5 h. A solution of 1-fluoro-3-nitrobenzene (19.25 g) in THF (25 mL) was added in a single portion. The cooling bath was removed and the mixture was heated at 80° C. for 4 h, then at room temperature overnight. The mixture was cooled in an ice bath and was stirred during the addition of water (500 mL). The resulting mixture was extracted into EtOAc (2×500 mL). The combined organic extracts were washed with brine (250 mL), dried over magnesium sulphate and evaporated in vacuo. The crude material was purified by ISCO Companion silica chromatography, eluting with a gradient 0-5% EtOAc in cyclohexane to give the title compound.

¹H NMR (CDCl₃) δ 8.05-8.02 (1H, m), 7.85-7.80 (1H, m), 7.45-7.41 (2H, m), 4.23 (2H, q), 2.15 (3H, s), 1.39 (3H, t).

### Intermediate 99

### -O-(3-Nitrophenyl)hydroxylamine

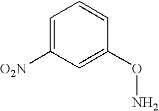

A solution of Intermediate 98 (18.0 g) in 1,4-dioxane (75 mL) was stirred at 0° C. and 70% HClO₄ (55 mL) was added dropwise with stirring. After the addition was complete, the cooling bath was removed and stirring was continued for a further 2 h. The mixture was poured into ice/water (1200 mL) and was set aside until all the ice has melted. The mixture was basified to >pH 11 with 2M NaOH and was extracted with EtOAc (2×750 mL). The combined organic extracts were washed with brine (500 mL), dried over magnesium sulphate and were evaporated in vacuo. The crude material was purified by ISCO Companion silica chromatography, eluting with a gradient 0-100% EtOAc in cyclohexane to give the title compound.

¹H NMR (CDCl₃) δ 8.12-8.09 (1H, m), 7.84-7.80 (1H, m), 7.43-7.40 (2H, m), 6.03 (2H, br).

### Intermediate 100

### (1E)-1-(4-Bromophenyl)ethanone O-(3-nitrophenyl)oxime

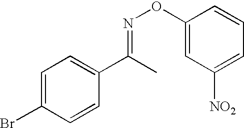

A mixture of Intermediate 99 (4.62 g) and 4-bromoacetophenone (5.97 g) in ethanol (100 mL) was stirred at room temperature and treated with concentrated HCl (3 drops). After ca. 5 mins a solid precipitated from the mixture. Stirring was continued for a further 30 mins. The solid was collected by filtration, and was washed with cold ethanol and cyclohexane to give the title compound.

¹H NMR (d₆-DMSO) δ 8.07 (1H, m), 7.94 (1H, m), 7.81 (2H, d), 7.75-7.65 (4H, m), 2.49 (3H, s).

### Intermediate 101

### 2-(4-Bromophenyl)-6-nitro-1-benzofuran

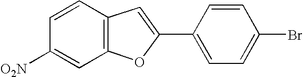

A mixture of Intermediate 100 (0.25 g), ethanol (10 mL) and concentrated HCl (3 mL) was stirred and heated at 80° C. for 2 days and then at room temperature for 4 days. The solid was collected by filtration, washed with cold ethanol and cyclohexane and dried in vacuo. The crude material was dissolved in THF and was loaded onto an ISCO Companion silica chromatography cartridge, which was sucked dry before eluting with a gradient 0-30% EtOAc in cyclohexane to give the title compound.

¹H NMR (d₆-DMSO) δ 8.57 (1H, d), 8.19 (1H, dd), 7.95 (2H, d), 7.91 (1H, d) 7.78 (2H, d), 7.75 (1H, s).

### Intermediate 102

### 2-(4-Bromophenyl)-1-benzofuran-6-amine

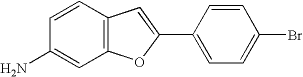

To a solution of 2-(4-bromophenyl)-6-nitro-1-benzofuran (420 mg, a synthesis of which is described above as Intermediate 101) in THF (25 mL) was added tin (II) chloride (1.73 g) and concentrated HCl (5 drops). The mixture was stirred at room temperature for 3 h. A further quantity of tin (II) chloride (1.73 g) was added and stirring at room temperature was continued for 18 h. A third batch of tin (II) chloride (1.73 g) was added together with concentrated HCl (5 drops) and the resulting mixture was stirred at room temperature for a further 24 h. The mixture was diluted to 150 mL with water, basified with 2M sodium hydroxide solution, and extracted with EtOAc (2×150 mL). The biphasic mixture was filtered through a pad of Celite to remove insoluble tin residues. The organic layer was collected, washed with saturated brine (50 mL), dried (MgSO₄) and evaporated in vacuo to give the title compound.

MS calcd for (C₁₄H₁₀BrNO+H)⁺: 288/290

MS found (electrospray): (M+H)⁺=288/290

### Intermediate 103

### 2-[4-(4,4,5,5-Tetramethyl-1,3,2-dioxaborolan-2-yl)phenyl]-1-benzofuran-6-amine

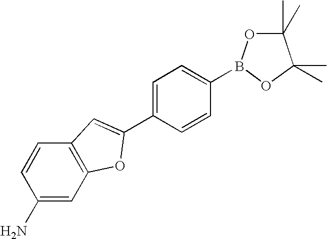

A solution of Intermediate 102 (210 mg) in 1,4-dioxane (10 mL) was treated with bis(pinacolato)diboron (277 mg), potassium acetate (216 mg) and PdCl₂(dppf) (40 mg). The mixture was heated at 100° C. under nitrogen for 4 h. The solvent was removed in vacuo and the residue was partitioned between water (100 mL) and DCM (100 mL). The organics were separated, dried by hydrophobic frit and evaporated in vacuo. The crude material was purified by ISCO Companion silica chromatography, eluting with a gradient 0-100% EtOAc in cyclohexane to give the title compound.

MS calcd for (C₂₀H₂₂BNO₃+H)⁺: 336

MS found (electrospray): (M+H)⁺=336

### Intermediate 104

### Methyl 5-[4-(6-amino-1-benzofuran-2-yl)phenyl]-3-[[(trans-4-methylcyclohexyl)carbonyl](1-methylethyl)amino]-2-thiophenecarboxylate

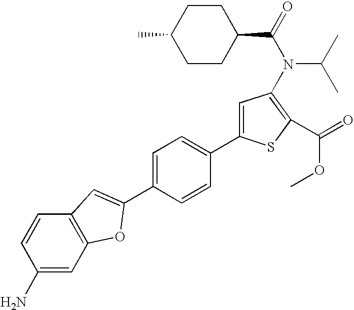

A mixture of Intermediate 103 (150 mg), Intermediate 5 (125 mg, prepared by method B), tetrakis(triphenylphosphine)palladium (0) (45 mg) and 2M sodium carbonate solution (1 mL) in DMF (2 mL) was stirred under nitrogen at 100° C. for 2 h. The mixture was allowed to cool and was evaporated in vacuo. The residue was partitioned between water (100 mL) and EtOAc (100 mL). The organics were separated, dried over magnesium sulphate and were evaporated in vacuo. The crude material was purified by ISCO Companion silica chromatography, eluting with a gradient 0-100% EtOAc in cyclohexane to give the title compound.

MS calcd for (C₃₁H₃₄N₂O₄S+H)⁺: 531

MS found (electrospray): (M+H)⁺=531

### Intermediate 105

### Methyl 3-{[(trans-4-methylcyclohexyl)carbonyl]amino}-2-thiophenecarboxylate

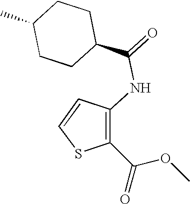

To a stirred solution of methyl 3-amino-2-thiophenecarboxylate (13.2 g) in dry DCM (211 mL) was added triethylamine (12.75 g), followed by dropwise addition of trans-4-methylcyclohexanecarbonyl chloride¹ (20.24 g). The reaction was stirred under nitrogen at room temperature for 16 h. The mixture was washed with saturated sodium bicarbonate solution and the organic phase was extracted with DCM (×3). The organics were separated using a hydrophobic frit and were evaporated in vacuo. The crude material was purified by silica Biotage cartridge, eluting with 5% EtOAc in cyclohexane to give the title compound.

MS calcd for (C₁₄H₁₉NO₃S+H)⁺: 282

MS found (electrospray): (M+H)⁺=282

Ref 1: WO 2004/052885

### Intermediate 106

### Methyl 3-{(ethyl[(trans-4-methylcyclohexyl)carbonyl]amino}-2-thiophenecarboxylate

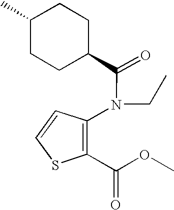

To a stirred suspension of methyl 3-{[(trans-4-methylcyclohexyl)carbonyl]amino}-2-thiophenecarboxylate (5.03 g, a synthesis of which is described above as Intermediate 105) in dry DMF (60 mL) under nitrogen was added sodium hydride (0.79 g, 60% dispersion in mineral oil) portion-wise over 5 mins. The mixture was stirred for 2 h at room temperature before iodoethane (1.59 mL) was added. The reaction was allowed to stir at room temperature for 23 h and was evaporated in vacuo. The residue was partitioned between DCM and water and the organics were separated using a hydrophobic frit. The organics were evaporated in vacuo and the crude material was purified by ISCO Companion silica chromatography, eluting with a gradient 0-30% EtOAc in cyclohexane to give the title compound.

MS calcd for (C₁₆H₂₃NO₃S+H)⁺: 310

MS found (electrospray): (M+H)⁺=310

### Intermediate 107

### 2-Bromo-1-(5-bromo-2-pyridinyl)ethanone

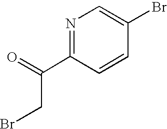

To a solution of 1-(5-bromo-2-pyridinyl)ethanone (300 mg) in dry DCM (5 mL) at 0° C. under nitrogen was added 2,6-lutidine (0.21 mL) and trimethylsilytriflate (0.27 mL). The reaction was stirred at 0° C. under nitrogen for 30 mins. N-Bromosuccinimide (270 mg) was added and the mixture was stirred at room temperature under nitrogen for 30 mins. Water (15 mL) and 2M HCl (5 drops) were added and the organics were extracted into DCM (2×13 mL). The organics were separated, dried using a hydrophobic frit and evaporated in vacuo. The crude material was purified by ISCO Companion silica chromatography, eluting with a gradient 0-100% EtOAc in cyclohexane to give the title compound.

MS calcd for (C₇H₅Br₂NO+H)⁺: 279/280/282

MS found (electrospray): (M+H)⁺=279/280/282

### Intermediate 108

### 6-(5-Bromo-2-pyridinyl)imidazo[2,1-b][1,3]thiazole

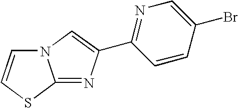

A solution of Intermediate 107 (370 mg) and 2-aminothiazole (120 mg) in acetone (30 mL) was heated at reflux for 2 h. A precipitate formed which was filtered off, dissolved in ethanol (20 mL) and 2M HCl (20 mL) and was heated at 70° C. for 3 days. The ethanol was removed in vacuo and the aqueous solution was basified with sodium carbonate solution and was filtered. The solid was washed with water and dried to give the title compound.

¹H NMR (d₆-DMSO) δ 8.66 (1H, s), 8.31 (1H, s), 8.07 (1H, d), 7.95 (1H, br), 7.89 (1H, d), 7.37 (1H, br).

### Intermediate 109

### Methyl 5-(6-imidazo[2,1-b][1,3]thiazol-6-yl-3-pyridinyl)-3-[[(trans-4-methylcyclohexyl)carbonyl](1-methylethyl)amino]-2-thiophenecarboxylate

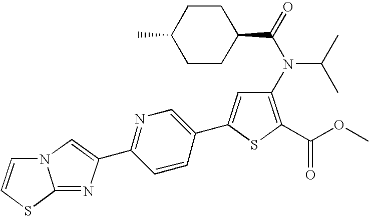

A mixture of Intermediate 64 (268 mg), Intermediate 108 (102 mg), sodium carbonate (116 mg) and tetrakis(triphenylphosphine)palladium (0) (50 mg) in 1,4-dioxane (1.5 mL) and water (0.2 mL) was heated in a microwave at 110° C. for 20 mins. The reaction mixture was evaporated in vacuo and was partitioned between DCM and sodium bicarbonate solution. The organics were separated and the aqueous was extracted with DCM (×2). The combined organics were dried by hydrophobic frit and evaporated in vacuo. The crude material was purified by ISCO Companion silica chromatography, eluting with a gradient 0-100% MeOH in DCM to give the title compound.

MS calcd for (C₂₇H₃₀N₄O₃S₂+H)⁺: 523

MS found (electrospray): (M+H)⁺=523

### Intermediate 110

### (2E/Z)-3-Amino-3-(4-bromo-2-chlorophenyl)-2-propenenitrile

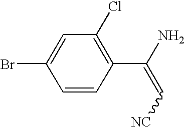

4-Bromo-2-chlorobenzonitrile (2.16 g) was dissolved in toluene (40 mL) and treated with acetonitrile (1.02 mL). Potassium tert-butoxide (3.37 g) was added and the mixture stirred under nitrogen for 1 h. Toluene (40 mL) was added and the mixture was stirred at room temperature for 3 days. Diethyl ether (150 mL) and saturated sodium bicarbonate solution (100 mL) were added and the mixture stirred vigorously for 10 min. The organic layer was separated, dried over sodium sulphate and concentrated. The residue was triturated with dry diethyl ether and the solid filtered to give the title compound.

¹H NMR (CDCl₃) δ 7.63 (1H, d), 7.47 (1H, dd), 7.26 (1H, d), 4.94 (2H, br), 4.07 (1H, s)

### Intermediate 111

### 3-(4-Bromo-2-chlorophenyl)-3-oxopropanenitrile

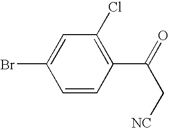

Intermediate 110 (1.5 g) was dissolved in chloroform (150 mL) and treated with 3N HCL solution (100 mL). The mixture was stirred vigorously at room temperature for 2 days. The chloroform layer was separated using a hydrophobic frit and concentrated. The crude material was purified by ISCO Companion silica chromatography, eluting with a gradient 3-100% EtOAc in cyclohexane to give the title compound.

MS calcd for (C₉H₅BrClNO—H)⁺: 256/258/260

MS found (electrospray): (M−H)⁺=256/258/260

### Intermediate 112

### 3-(4-Bromo-2-chlorophenyl)-1H-pyrazol-5-amine

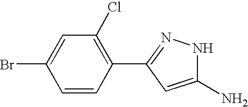

Intermediate 111 (1.3 g) was dissolved in ethanol (50 mL) and acetic acid (2 mL) was added. The mixture was treated with hydrazine monohydrate (0.73 mL) and the mixture heated at reflux for 6 h. The solvent was removed in vacuo and the residue partitioned between DCM and saturated sodium bicarbonate solution. The DCM layer was separated using a hydrophobic frit and concentrated to give the title compound.

MS calcd for (C₉H₇BrClN₃—H)⁺: 272/274/276

MS found (electrospray): (M−H)⁺=272/274/276

### Intermediate 113

### 2-(4-Bromo-2-chlorophenyl)pyrazolo[1,5-a]pyrimidin-7-amine

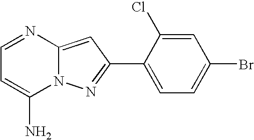

Intermediate 112 (1.38 g) was dissolved in acetic acid (20 mL) and was treated with 3-dimethylaminoacrylonitrile (0.433 mL). The mixture was heated at 110° C. for 1.5 h. The acetic acid was removed in vacuo and the residue was triturated with DCM. The solid was filtered off to give the title compound.

MS calcd for (C₁₂H₈BrClN₄+H)⁺: 322/324/326

MS found (electrospray): (M+H)⁺=322/324/326

### Intermediate 114

### 2-[2-Chloro-4-(4,4,5,5-tetramethyl-1,3,2-dioxaborolan-2-yl)phenyl]pyrazolo[1,5-a]pyrimidin-7-amine

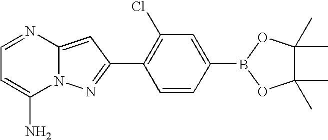

A solution of Intermediate 113 (500 mg) in 1,4-dioxane (15 mL) was treated with bis(pinacolato)diboron (786 mg), potassium acetate (608 mg) and PdCl₂(dppf) (113 mg). The mixture was heated at 100° C. under nitrogen for 24 h. Further portions of bis(pinacolato)diboron (786 mg), potassium acetate (608 mg) and PdCl₂(dppf) (113 mg) were added and the mixture was heated at 100° C. for 4 days. The reaction was cooled and concentrated in vacuo. The residue was partitioned between DCM and water. The organics were separated and the aqueous re-extracted with DCM (×2). The combined organics were dried using a hydrophobic frit and were concentrated in vacuo. The crude material was purified by silica SPE cartridge, eluting with a gradient 10-100% EtOAc in cyclohexane to give the title compound as a 55:45 ratio with [4-(7-aminopyrazolo[1,5-a]pyrimidin-2-yl)-3-chlorophenyl]boronic acid.

Boronate ester:

MS calcd for (C₁₈H₂₀BClN₄O₂+H)⁺: 371

MS found (electrospray): (M+H)⁺=371

Boronic acid:

MS calcd for (C₁₂H₁₀BClN₄O₂+H)⁺: 289

MS found (electrospray): (M+H)⁺=289

### Intermediate 115

### Methyl 5-[4-(7-aminopyrazolo[1,5-a]pyrimidin-2-yl)-3-chlorophenyl]-3-[[(trans-4-methylcyclohexyl)carbonyl](1-methylethyl)amino]-2-thiophenecarboxylate

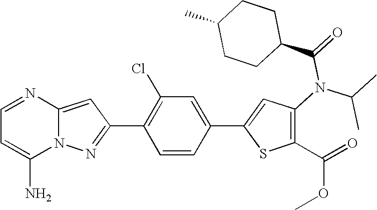

Methyl 5-iodo-3-[[(trans-4-methylcyclohexyl)carbonyl] (1-methylethyl)amino]-2-thiophene carboxylate (400 mg, a synthesis of which is described above as Intermediate 5, method B) was dissolved in DMF (10 mL) and was treated with Intermediate 114 (248 mg), sodium carbonate (284 mg) in water (3 mL), followed by tetrakis(triphenylphosphine)palladium (0) (104 mg). The mixture was heated at 100° C. for 2.5 h. The mixture was allowed to cool to room temperature and the solvent was evaporated in vacuo. The residue was partitioned between DCM and water, the organics were separated using a hydrophobic frit and concentrated. The crude material was purified by ISCO Companion silica chromatography, eluting with a gradient 2-100% EtOAc in cyclohexane to give the title compound.

MS calcd for (C₂₉H₃₂ClN₅O₃S+H)⁺: 566

MS found (electrospray): (M+H)⁺=566

### Intermediate 116

### (2Z)-3-Amino-3-(5-bromo-2-pyridinyl)-2-propenenitrile

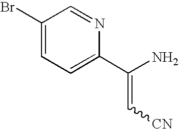

To a solution of 5-bromo-2-pyridinecarbonitrile (1.83 g) in toluene (40 mL) and MeCN (1.04 mL) was added potassium tert-butoxide (3.37 g). The mixture was stirred under nitrogen at room temperature for 3 days. The mixture was partitioned between Et₂O and sodium bicarbonate solution. The organics were separated and dried over magnesium sulphate, then filtered and evaporated in vacuo. The residue was purified by ISCO Companion silica chromatography, eluting with a gradient 0-40% EtOAc in cyclohexane to give the title compound.

MS calcd for (C₈H₆N₃Br+H)⁺: 224/226

MS found (electrospray): (M+H)⁺=224/226

### Intermediate 117

### 3-(5-Bromo-2-pyridinyl)-1H-pyrazol-5-amine hydrochloride

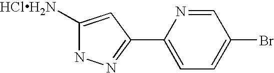

Intermediate 116 (600 mg) and hydrazine monohydrate (252 mg) were heated in isopropyl alcohol (12 mL) at 90° C. for 4 h. 2N HCl (2 mL) was added to the mixture at 90° C. and the reaction was then left to cool to room temperature overnight. A precipitate formed which was filtered off and washed with isopropyl alcohol to give the title compound.

MS calcd for (C₈H₇N₄Br.HCl+H)⁺: 239/241

MS found (electrospray): (M+H)⁺=239/241

### Intermediate 118

### 2-(5-Bromo-2-pyridinyl)pyrazolo[1,5-a]pyrimidine

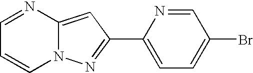

3-(5-Bromo-2-pyridinyl)-1H-pyrazol-5-amine hydrochloride (625 mg, a synthesis of which is described above as intermediate 117), 1,1,3,3-tetrakis(methyloxy)propane (0.448 mL) and acetic acid (9 mL) were heated at 110° C. for 2.5 h. The reaction was allowed to cool to room temperature overnight and a brown precipitate formed. The mixture was diluted with water (10 mL) and was filtered. The filter cake was washed with water, dried by air flow and then in a vacuum oven at 40° C. overnight to give the title compound.

MS calcd for (C₁₁H₇N₄Br+H)⁺: 275/277

MS found (electrospray): (M+H)⁺=275/277

### Intermediate 119

### Methyl 3-[[(trans-4-methylcyclohexyl)carbonyl](1-methylethyl)amino]-5-(6-pyrazolo[1,5-a]pyrimidin-2-yl-3-pyridinyl)-2-thiophenecarboxylate

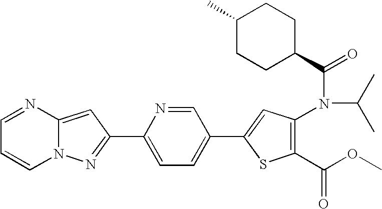

Tetrakis(triphenylphosphine)palladium (0) (40 mg) was added to a mixture of {4-[[(trans-4-methylcyclohexyl)carbonyl](1-methylethyl)amino]-5-[(methyloxy)carbonyl]-2-thienyl}boronic acid (140 mg, a synthesis of which is described above as Intermediate 64), 2-(5-bromo-2-pyridinyl)pyrazolo[1,5-a]pyrimidine (98.8 mg, a synthesis of which is described above as Intermediate 118) and potassium phosphate (22 mg) in 1,4-dioxane (3 mL) and water (1 mL). The mixture was heated at 100° C. under nitrogen for 3 h. On cooling the reaction was evaporated in vacuo, and the residue was partitioned between DCM (10 mL) and 8% sodium bicarbonate solution (2×10 mL). The organics were separated by hydrophobic frit and were evaporated in vacuo. The crude material was purified by silica SPE cartridge, eluting with a gradient 10-100% EtOAc in cyclohexane. The product was then dissolved in MeOH and was applied to an SCX-2 cartridge, which was eluted with MeOH (3 volumes) and 10% 0.88 ammonia solution in MeOH (3 volumes) to give the title compound.

MS calcd for (C₂₈H₃₁N₅O₃S+H)⁺: 518

MS found (electrospray): (M+H)⁺=518

### Intermediate 120

### 5-Bromo-2-ethynylpyridine

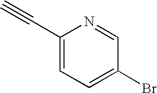

2,5-Dibromopyridine (2 g), (trimethylsilyl)acetylene (1.19 mL) and copper (I) iodide (38 mg) were dissolved in triethylamine (26 mL) with ice cooling. The reaction mixture was purged with nitrogen, then bis(triphenylphosphine)palladium (II) chloride (140 mg) was added in one portion. The mixture was stirred and cooled in ice for 1 h then stirred at room temperature for 1 h under and atmosphere of nitrogen. The mixture was diluted with Et₂O and was washed with water (×3). The organics were separated, dried over sodium sulphate, filtered and evaporated in vacuo. This material was purified by silica SPE cartridge, eluting with cyclohexane, then 3:1 cyclohexane in DCM. Product containing fractions were evaporated in vacuo and the residue was dissolved in pentane. Cooling in ice gave a precipitate which was filtered off. The filtrate was evaporated in vacuo and the residue was dissolved in MeOH (18 mL) and was treated with 2M NaOH (3 mL). This mixture was stirred for 30 mins and was then quenched with acetic acid (0.36 mL) and was evaporated in vacuo until a precipitate was formed. The mixture was diluted with Et₂O and was washed with water. The organic phase was separated, dried over sodium sulphate, filtered and evaporated in vacuo. The crude material was purified by silica SPE cartridge, eluting with 3:1 cyclohexane in DCM to give the title compound.

MS calcd for (C₇H₄Br+H)⁺: 182/184

MS found (electrospray): (M+H)⁺=182/184

### Intermediate 121

### 2-(5-Bromo-2-pyridinyl)furo[3,2-b]pyridine

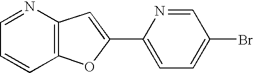

Intermediate 120 (590 mg), 2-iodo-3-pyridinol (716 mg) and copper (I) iodide (79 mg) were mixed in triethylamine (13 mL). The stirred solution was degassed and purged with nitrogen. Bis(triphenylphosphine)palladium (II) chloride (202 mg) was added and the reaction was stirred at 90° C. for 1.75 h. After cooling to room temperature, the volume of solvent was reduced in vacuo and the residue was partitioned between EtOAc and water (some insoluble material remained). The organic phase was separated, dried over sodium sulphate, filtered and evaporated in vacuo. The residue was dissolved in DCM and was purified by silica SPE cartridge, eluting with DCM, 2:1 cyclohexane/EtOAc, Et₂O and EtOAc. The product containing fractions were evaporated in vacuo and as the solvent volume reduced a precipitate formed. The precipitate was filtered off and dried in vacuo to give the title compound.

MS calcd for (C₁₂H₇BrN₂O+H)⁺: 275/277

MS found (electrospray): (M+H)⁺=275/277

### Intermediate 122

### Methyl 5-(6-furo[3,2-b]pyridin-2-yl-3-pyridinyl)-3-[[(trans-4-methylcyclohexyl)carbonyl](1-methylethyl)amino]-2-thiophenecarboxylate

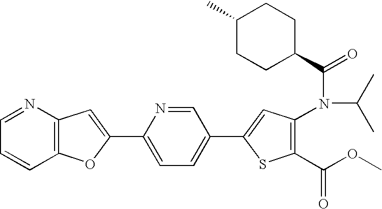

Tetrakis(triphenylphosphine)palladium (0) (40 mg), was added to a mixture of {4-[[(trans-4-methylcyclohexyl)carbonyl](1-methylethyl)amino]-5-[(methyloxy)carbonyl]-2-thienyl}boronic acid (140 mg, a synthesis of which is described above as Intermediate 64), Intermediate 121 (99 mg) and potassium phosphate (222 mg) in 1,4-dioxane (3 mL) and water (1 mL). The reaction was heated at 100° C. for 3 h and was then allowed to cool and was evaporated in vacuo. The residue was partitioned between DCM (10 mL) and 8% sodium bicarbonate solution (2×10 mL). The organics were separated, dried using a hydrophobic frit and evaporated in vacuo. The crude material was purified by MDAP HPLC to give the title compound.

MS calcd for (C₂₉H₃₁N₃O₄S+H)⁺: 518

MS found (electrospray): (M+H)⁺=518

### Intermediate 123

### Methyl 3-[[(2,4-dichlorophenyl)carbonyl](1-methylethyl)amino]-2-thiophenecarboxylate

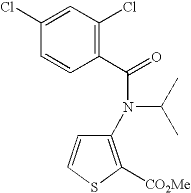

To a solution of methyl 3-[(1-methylethyl)amino]-2-thiophenecarboxylate (a synthesis of which is described in Intermediate 20) (5 g) in pyridine (45 mL) was added 2,4-dichlorobenzoyl chloride (5.3 mL). The reaction mixture was heated at 70° C. under nitrogen overnight. The reaction mixture was partitioned between EtOAc and sodium bicarbonate solution. The organics were washed with brine, dried with sodium sulphate and evaporated to dryness. This was purified by ISCO companion silica chromatography eluting with a gradient of ethyl acetate in cyclohexane to give the title compound.

MS calcd for (C₁₆H₁₅NO₃SCl₂+H)⁺: 372/373/374

MS found (electrospray): (M+H)⁺=372/373/374

### Intermediate 124

### Methyl 3-[[(2,4-dichlorophenyl)carbonyl](1-methylethyl)amino]-5-iodo-2-thiophenecarboxylate

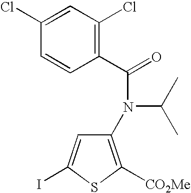

LDA (9.5 mL) was cooled to −78° C. under nitrogen. A solution of Intermediate 123 (3.0 g) in anhydrous THF (30 mL) was added dropwise keeping the internal temperature between −73° C. and −65° C. The reaction mixture was stirred at −74° C. for 15 min. A solution of iodine (6.1 g) in anhydrous THF (30 mL) was added dropwise over −20 min maintaining the internal temperature <−65° C. Sat. NH₄Cl (aq) (10 mL) was added dropwise after 15 min and the reaction mixture was warmed to room temperature before washing with 5% sodium thiosulphate solution. The organic was separated and the aqueous extracted with EtOAc (×2). The combined organics were dried with sodium sulphate and evaporated to dryness. The residue was purified by ISCO companion silica chromatography eluting with a gradient of ethyl acetate in cyclohexane to give the title compound.

MS calcd for (C₁₆H₁₄NO₃SCl₂I+H)⁺: 498/500/502

MS found (electrospray): (M+H)⁺=498/500/502

### Intermediate 125

### 5-(4-Bromophenyl)-2-(triphenylmethyl)-2H-tetrazole

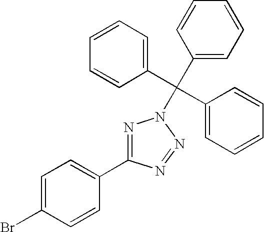

A mixture of 5-(4-bromophenyl)-1H-tetrazole (2 g, 8.89 mmol), trityl chloride (2.47 g, 8.89 mmol), tetrabutyl ammonium bromide (130 mg) and 2N sodium hydroxide solution (4.79 mL, 9.33 mmol) in DCM (15 mL) was stirred vigorously at RT for 3 h. The mixture was diluted with DCM (50 mL) and water (30 mL). The layers were separated and the aqueous extracted further with DCM (3×20 mL). The combined organic fractions were dried (hydrophobic frit) and evaporated to give a solid. This was applied in DCM to a 20 g SPE (silica) and eluted with DCM gave the title compound.

¹H NMR (400 MHz, CDCl₃) δ 8.05 (2H, d), 7.6 (2H, d), 7.4-7.1 (15H, m).

### Intermediate 126

### Methyl 3-[[(2,4-dichlorophenyl)carbonyl](1-methylethyl)amino]-5-[4-(1H-tetrazol-5-yl)phenyl]-2-thiophenecarboxylate

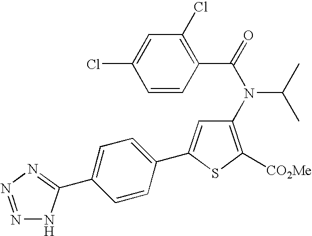

n-Butyl lithium (2.94 mL, 4.7 mmol, 1.6M solution in hexanes) was added dropwise to a solution of Intermediate 125 (2.0 g) in THF (25 mL) at −78° C. under nitrogen. A fine suspension was formed and stirred at −78° C. under nitrogen for 45 mins. Trimethoxyborate (669 μL, 5.99 mmol) in THF (3 mL) was added dropwise, maintaining the internal temp at −78° C. This was stirred at −78° C. for 30 mins then allowed to warm to RT and stirred for a further 45 mins. Water (30 mL) was added slowly and the mixture extracted with EtOAc (2×40 mL), dried (Na₂SO₄) and evaporated to give an oil. This was triturated with diethyl ether (40 mL) and the resulting solid (A) was filtered off. Tetrakis(triphenylphosphine)palladium (0) (23 mg, 0.02 mmol) was added to a mixture of Intermediate 124 (200 mg, 0.40 mmol) and the solid (A) (185 mg, 0.40 mmol) in dioxan (3 mL)/2N sodium carbonate solution (1 mL) and heated to 100° C. under nitrogen for 4 h. The reaction was allowed to cool and the solvent evaporated, acidified with 2N HCl and the phases separated using an aqueous extraction cartridge. This was evaporated to give a solid, which was applied in the minimum volume of DCM to a 20 g Si SPE cartridge. This was eluted with cyclohexane then cyclohexane/EtOAc [(9:1) gradient to (7:3)]. Further elution with EtOAc to MeCN to acetone to MeOH. These latter fractions were combined and evaporated, dissolved in MeOH and applied to a 5 g NH₂ SPE cartridge. Elution was with MeOH (5× column volumes) then 10% acetic acid/MeOH (5× column volumes). The acetic acid/MeOH fractions were combined and evaporated to give the title compound.

MS calcd for (C₂₃H₁₉Cl₂N₅O₃S+H)⁺: 516/518/520

MS found (electrospray): (M+H)⁺=516/518/520

### Intermediate 127

### Methyl 3-[(cyclopropylmethyl)amino]-2-thiophenecarboxylate

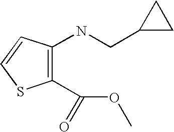

To a stirred solution of methyl 3-amino-2-thiophenecarboxylate (1.44 g) in DCM (25 mL) was added glacial acetic acid (1.57 mL) and cyclopropanecarbaldehyde (707 mg). Sodium triacetoxyborohydride (3.88 g) was added portionwise and the reaction was stirred at room temperature under nitrogen overnight. Water (30 mL) was added carefully to the reaction mixture, dropwise at first. The solution was then neutralised with sodium carbonate. The organics were separated and the aqueous was extracted with DCM. The combined organics were separated using a hydrophobic frit and were evaporated in vacuo. The crude material was purified by ISCO Companion silica chromatography, eluting with a gradient 5-20% EtOAc in cyclohexane to give the title compound.

MS calcd for (C₁₀H₁₃NO₂S+H)⁺: 212

MS found (electrospray): (M+H)⁺=212

### Intermediate 128

### Methyl 3-{(cyclopropylmethyl)[(trans-4-methylcyclohexyl)carbonyl]amino}-2-thiophenecarboxylate

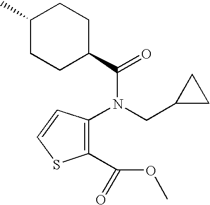

To a solution of Intermediate 127 (1.83 g) in DCE (35 mL) was added trans-4-methylcyclohexanecarbonyl chloride¹ (1.6 g). The mixture was heated at 86° C. under nitrogen for 18 h and was then allowed to cool to room temperature. The reaction was quenched with saturated sodium bicarbonate solution. The organic layer was separated and the aqueous layer was extracted with DCM. The combined organics were separated using a hydrophobic frit and were evaporated in vacuo. The crude material was purified by ISCO Companion silica chromatography, eluting with a gradient 5-100% EtOAc in cyclohexane to give the title compound.

MS calcd for (C₁₈H₂₅NO₃S+H)⁺: 336

MS found (electrospray): (M+H)⁺=336

Ref 1: WO 2004/052885

### Intermediate 129

### Methyl 3-{((cyclopropylmethyl)[(trans-4-methylcyclohexyl)carbonyl]amino}-5-iodo-2-thiophenecarboxylate

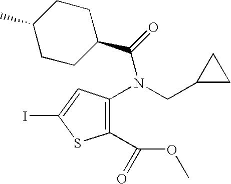

A solution of LDA (1.8M solution in THF/heptane/ethyl benzene, 12.1 mL) was cooled to −78° C. under an atmosphere of nitrogen. Intermediate 128 (2.44 g) was dissolved in dry THF (30 mL) and was added dropwise over 15 mins, maintaining an internal temperature between −78° C. and −71° C. The reaction mixture was then stirred for 30 mins at −78° C. Iodine (3.68 g) in dry THF (30 mL) was then added dropwise maintaining an internal temperature between −78° C. and −70° C. The reaction mixture was stirred at −78° C. for 10 mins before being slowly quenched with saturated ammonium chloride solution (45 mL). The reaction was allowed to warm to room temperature before being washed with 5% sodium thiosulphate solution. The organic phase was separated and the aqueous phase was extracted with EtOAc (×2). The combined organics were dried by passing through a hydrophobic frit and were evaporated in vacuo. The crude material was purified by ISCO Companion silica chromatography, eluting with a gradient 5-100% EtOAc in cyclohexane to give the title compound.

MS calcd for (C₁₈H₂₄INO₃S+H)⁺: 462

MS found (electrospray): (M+H)⁺=462

### Intermediate 130

### Methyl 3-{(cyclopropylmethyl)[(trans-4-methylcyclohexyl)carbonyl]amino}-5-(4-pyrazolo[1,5-a]pyrimidin-2-ylphenyl)-2-thiophenecarboxylate

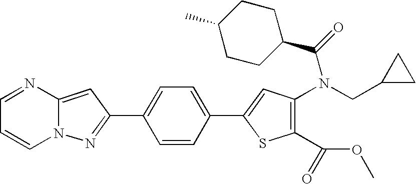

A mixture of Intermediate 129 (299 mg), 2-[4-(4,4,5,5-tetramethyl-1,3,2-dioxaborolan-2-yl)phenyl]pyrazolo[1,5-a]pyrimidine (313 mg, a synthesis of which is described above as Intermediate 10), 1 M sodium carbonate solution (3.56 mL) and tetrakis(triphenylphosphine)palladium (0) (104.8 mg) in DMF (7 mL) was heated at 100° C. under nitrogen for 3 h and was then stirred at room temperature for 18 h. The reaction was evaporated in vacuo and the residue was partitioned between EtOAc and water. The organics were separated and the aqueous layer was extracted into EtOAc. The combined organics were dried by passing through a hydrophobic frit and were evaporated in vacuo to give the title compound.

MS calcd for (C₃₀H₃₂N₄O₃S+H)⁺: 529

MS found (electrospray): (M+H)⁺=529

### Intermediate 131

### (2Z)-3-Amino-3-(5-bromo-2-thienyl)-2-propenenitrile

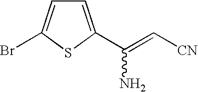

Potassium tert-butoxide (5.37 g) was added in portions to a solution of 5-bromo-2-thiophenecarbonitrile (3.0 g) in dry toluene (60 mL) and dry MeCN (1.66 mL) at room temperature under nitrogen. The solution was left to stir at room temperature for 2.5 h. Diethyl ether (100 mL) and saturated sodium carbonate solution (100 mL) were added and the mixture was stirred vigorously for 5 mins. The layers were separated and the aqueous extracted further with diethyl ether (2×50 mL). The combined organics were dried over sodium sulphate and were evaporated to give the title compound.

MS calcd for (C₇H₅BrN₂S+H)⁺: 229/331

MS found (electrospray): (M+H)⁺=229/331

### Intermediate 132

### 3-(5-Bromo-2-thienyl)-3-oxopropanenitrile

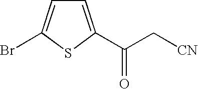

Intermediate 131 (3.58 g) was dissolved in chloroform (200 mL) and was treated with 5N HCl (200 mL). The mixture was stirred vigorously for 24 h. The mixture was separated using a hydrophobic frit and the organics were evaporated to give the title compound.

MS calcd for (C₇H₄BrNOS—H)⁻: 228/330

MS found (electrospray): (M−H)⁻=228/330

### Intermediate 133

### 5-(5-Bromo-2-thienyl)-1H-pyrazol-3-amine

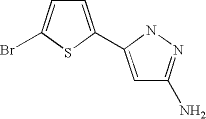

A suspension of Intermediate 132 (3.18 g) in absolute ethanol (70 mL) was treated with acetic acid (3.4 mL). Hydrazine monohydrate (1.34 mL) was added and the mixture was heated at 70° C. under nitrogen for 2 h. The reaction was allowed to cool and was poured into 8% sodium bicarbonate solution. The mixture was extracted with DCM (×2) and the combined organics were washed with water (2×50 mL), separated using a hydrophobic frit and evaporated in vacuo to give the title compound.

MS calcd for (C₇H₆BrN₃S+H)⁺: 244/246

MS found (electrospray): (M+H)⁺=244/246

### Intermediate 134

### 2-(5-Bromo-2-thienyl)pyrazolo[1,5-a]pyrimidine

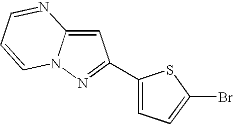

To a solution of Intermediate 133 (2.65 g) in acetic acid (25 mL) was added 1,1,3,3-tetrakis(methyloxy)propane (2.14 mL) at room temperature under nitrogen. The reaction was heated at 110° C. for 24 h. The reaction was allowed to cool and the solvent was evaporated. The residue was partitioned between chloroform (200 mL) and 8% sodium bicarbonate solution (100 mL). The organic layer was washed with further sodium bicarbonate solution (100 mL), was separated by hydrophobic frit and evaporated. The crude material was purified by silica SPE, eluting with a stepped gradient 10-50% EtOAc in cyclohexane to give the title compound.

MS calcd for (C₁₀H₆BrN₃S+H)⁺: 280/282

MS found (electrospray): (M+H)⁺=280/282

### Intermediate 135

### 2-(5-Iodo-2-thienyl)pyrazolo[1,5-a]pyrimidine

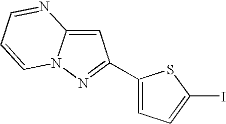

2-(5-Bromo-2-thienyl)pyrazolo[1,5-a]pyrimidine (535 mg, a synthesis of which is described above as Intermediate 134) was dissolved in 1,4-dioxane (10 mL). Sodium iodide (571 mg), copper (I) iodide (17 mg) and (1R,2R)-N,N′-dimethyl-1,2-cyclohexanediamine (0.028 mL) were added and the reaction was heated at 110° C. under nitrogen for 3 h. The reaction was cooled and set aside. 2-(5-Bromo-2-thienyl)pyrazolo[1,5-a]pyrimidine (1.98 g, a synthesis of which is described above as Intermediate 134) was dissolved in 1,4-dioxane (40 mL). Sodium iodide (2.11 g), copper (I) iodide (63 mg) and (1R,2R)-N,N′-dimethyl-1,2-cyclohexanediamine (0.103 mL) were added and the reaction was heated at 110° C. under nitrogen for 3 h. The reaction was cooled and was combined with the product from the above reaction. The combined material was poured into water (100 mL) and EtOAc (200 mL) and was stirred for 20 mins at room temperature. The layers were separated and the aqueous was extracted further with EtOAc (2×100 mL). The combined organics were dried over sodium sulphate and evaporated in vacuo. The crude material was purified by silica SPE cartridge, eluting with DCM to give the title compound.

MS calcd for (C₁₀H₆₁N₃S+H)⁺: 328

MS found (electrospray): (M+H)⁺=328

### Intermediate 136

### 4-Bromo-2-furancarboxylic acid

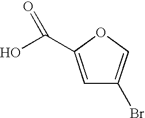

To a solution of 4-bromo-2-furancarbaldehyde (5 g) in tert-butanol (350 mL) was added 2-methyl-2-butene (100 mL) followed by dropwise addition over (30 mins) of a solution of sodium dihydrogenphosphate (23.9 g) and sodium chlorite (23 g) in water (165 mL). The reaction mixture was stirred at room temperature overnight. The reaction mixture was evaporated in vacuo and the residue was dissolved in water. The aqueous was extracted with cyclohexane (×2) and was then acidified to pH3 using 2N HCl. The aqueous was extracted with DCM (×3) and the combined DCM fractions were dried using a hydrophobic frit and evaporated in vacuo to give the title compound.

MS calcd for (C₅H₃BrO₃—H)—: 189/191

MS found (electrospray): (M−H)⁻=189/191

### Intermediate 137

### 2-(4-Bromo-2-furanyl)-1,3-benzoxazole

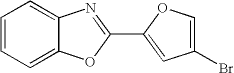

Intermediate 136 (1.5 g) was dissolved in DCM (20 mL). DEF (1 drop) was added followed by oxalyl chloride (1.04 mL). The reaction mixture was stirred at room temperature overnight, and was evaporated in vacuo to give 4-bromo-2-furancarbonyl chloride. 4-bromo-2-furancarbonyl chloride (300 mg, prepared above) and 2-aminophenol (156 mg) were dissolved in 1,4-dioxane (3 mL) and heated in the microwave at 210° C. for 45 min. This procedure was repeated 5 times. The reactions were combined and poured into 1N NaOH solution and this was extracted with DCM (×3). The organic was dried by passing through a hydrophobic frit and was evaporated in vacuo. This was purified by ISCO Companion silica chromatography eluting with a gradient 5-100% EtOAc in cyclohexane to give the title compound.

MS calcd for (C₁₁H₆NO₂Br+H)⁺: 264/266

MS found (electrospray): (M+H)⁺=264/266

### Intermediate 138

### [5-(1,3-Benzoxazol-2-yl)-3-furanyl]boronic acid

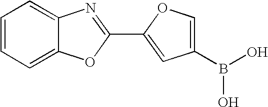

Intermediate 137 (422 mg) was dissolved in 1,4-dioxane (12 mL) then potassium acetate (459 mg), PdCl₂(dppf) (90 mg) and bis(pinacolato)diboron (568 mg) were added and the mixture was heated at 100° C. under nitrogen overnight. The reaction was cooled, evaporated in vacuo and partitioned between water and DCM. The organics were dried using a hydrophobic frit and evaporated in vacuo. The crude material was purified using a silica SPE cartridge, eluting with EtOAc, 1% MeOH in EtOAc and 5% MeOH in EtOAC. The material obtained was purified further by ISCO Companion silica chromatography, eluting with a gradient 5-100% EtOAc in cyclohexane to give the title compound as a 2:1 ratio with 2-[4-(4,4,5,5-tetramethyl-1,3,2-dioxaborolan-2-yl)-2-furanyl]-1,3-benzoxazole.

Boronic acid:

MS calcd for (C1H₈BNO₄+H)⁺: 230

MS found (electrospray): (M+H)⁺=230

Boronate ester:

MS calcd for (C₁₇H₁₈BNO₄+H)⁺: 312

MS found (electrospray): (M+H)⁺=312

### Intermediate 139

### Methyl 5-[5-(1,3-benzoxazol-2-yl)-3-furanyl]-3-[[(trans-4-methylcyclohexyl)carbonyl](1-methylethyl)amino]-2-thiophenecarboxylate

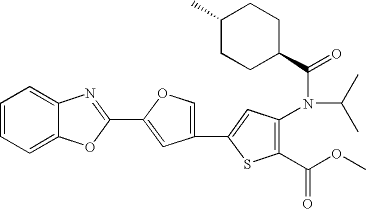

To a solution of methyl 5-iodo-3-[[(trans-4-methylcyclohexyl)carbonyl](1-methylethyl)amino]-2-thiophenecarboxylate (206 mg, a synthesis of which is described above as Intermediate 5) in DMF (10 mL) was added Intermediate 138 (105 mg), sodium carbonate (194 mg) in water (3 mL) and tetrakis(triphenylphosphine)palladium (0) (71 mg). The reaction was heated at 100° C. for 2 h and was evaporated in vacuo. The residue was partitioned between water and DCM and the organics were separated by hydrophobic frit. The organics were evaporated in vacuo and the residue was purified by ISCO Companion silica chromatography, eluting with a gradient 5-100% EtOAc in cyclohexane to give the title compound.

MS calcd for (C₂₈H₃₀N₂O₅S+H)⁺: 507

MS found (electrospray): (M+H)⁺=507

### Intermediate 140

### Ethyl (1S,2R,4S)-2-hydroxy-4-methylcyclohexanecarboxylate

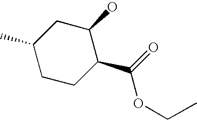

To a stirred solution of ethyl 4-methyl-2-oxocyclohexanecarboxylate (8.84 g) in dry MeOH (100 mL) at 2.5° C. under nitrogen was added sodium borohydride (2.72 g) portion-wise over 15 mins. The mixture was allowed to stir at room temperature for 2 h and was then acidified using 2N HCl. The solvent was concentrated in vacuo and the residue was partitioned between EtOAc and water. The organic phase was separated, dried over sodium sulphate and evaporated in vacuo. The crude material was purified by ISCO Companion silica chromatography, eluting with a gradient 5-30% EtOAc in cyclohexane to give the title compound.

¹H NMR (CDCl₃) δ 4.29-4.25 (1H, m), 4.17 (2H, q), 3.14 (1H, br), 2.36-2.29 (1H, m), 1.95-1.73 (5H, m), 1.10-0.91 (2H, M), 1.27 (3H, t), 0.88 (3H, d).

### Intermediate 141

### (1S,2R,4S)-2-Hydroxy-4-methylcyclohexanecarboxylic acid

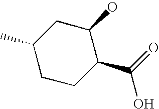

To a stirred solution of Intermediate 140 (0.75 g) in THF (5 mL) and ethanol (5 mL) was added 2N sodium hydroxide solution (2.5 mL). The solution was stirred at room temperature for 0.5 h. A precipitate formed after this time. The mixture was allowed to stand at room temperature for 20 h and was concentrated to approximately 25% volume in vacuo. The residue was partitioned between 2N HCl and DCM and the layers were separated. The aqueous was extracted with DCM (×2) and the combined organics were evaporated in vacuo to give the title compound.

¹H NMR (d₆-DMSO) δ 11.93 (1H, br), 4.18 (1H, d), 3.48 (1H, br), 2.18 (1H, dt), 1.78-1.49 (5H, m), 1.06 (1H, dt), 0.92-0.73 (4H, m).

### Intermediate 142

### (1S,2R,4S)-2-(Acetyloxy)-4-methylcyclohexanecarboxylic acid

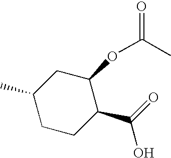

To a stirred solution of Intermediate 141 (0.49 g) in DCM (8 mL) was added pyridine (1.49 mL) followed by acetic anhydride (1.13 mL). The reaction was allowed to stand at room temperature for 23 h and was then evaporated in vacuo. The residue was partitioned between 2N HCl and DCM. The organic phase was separated using a hydrophobic frit and was evaporated in vacuo. The residue was dissolved in DCM (6 mL), then saturated sodium bicarbonate solution was added. The layers were stirred rapidly for 2 h. The aqueous was adjusted to pH1 using 2N HCl and then the organic phase was separated and evaporated in vacuo to give the title compound.

¹H NMR (CDCl₃) δ 5.55-5.40 (1H, m), 2.56-2.43 (1H, m), 2.04 (3H, s), 2.02-1.58 (5H, m), 1.19-1.09 (1H, m), 1.02-0.93 (1H, m), 0.90 (3H, d), carboxylic acid proton not seen.

### Intermediate 143

### Methyl 3-[(1-methylethyl)amino]-5-(4-pyrazolo[1,5-a]pyrimidin-2-ylphenyl)-2-thiophenecarboxylate

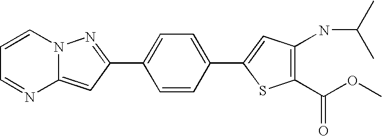

A mixture of methyl 5-iodo-3-[(1-methylethyl)amino]-2-thiophenecarboxylate (2.11 g, a synthesis of which is described above as Intermediate 4), 2-[4-(4,4,5,5-tetramethyl-1,3,2-dioxaborolan-2-yl)phenyl]pyrazolo[1,5-a]pyrimidine (999 mg, a synthesis of which is described above as Intermediate 10), in 1,4-dioxane (40 mL) was treated with sodium carbonate solution (1.09 g in water 20 mL) and tetrakis(triphenylphosphine)palladium (0) (150 mg). The reaction was heated at 100° C. under nitrogen for 2 h. A further portion of 2-[4-(4,4,5,5-tetramethyl-1,3,2-dioxaborolan-2-yl)phenyl]pyrazolo[1,5-a]pyrimidine (150 mg, a synthesis of which is described above as Intermediate 10) was added to the reaction and heating was continued for 1 h. The reaction was allowed to cool and was evaporated in vacuo. The residue was partitioned between DCM (50 mL) and 8% sodium bicarbonate solution (50 mL). The layers were separated and the aqueous extracted with DCM (3×40 mL). The organics were dried over sodium sulphate and were evaporated in vacuo. The residue was stirred in Et₂O (40 mL) for 20 mins. Solid was filtered off to give the title compound.

MS calcd for (C₂₁H₂₀N₄O₂S+H)⁺: 393

MS found (electrospray): (M+H)⁺=393

### Intermediate 144

### Methyl 3-[({[(1S,2R,4S)-2-(acetyloxy)-4-methylcyclohexyl]carbonyl}(1-methylethyl)amino]-5-(4-pyrazolo[1,5-a]pyrimidin-2-ylphenyl)-2-thiophenecarboxylate

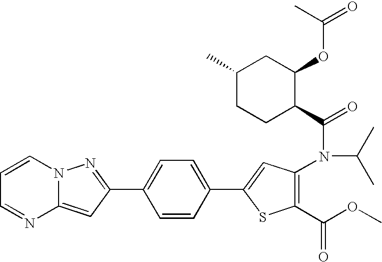

To a stirred solution of Intermediate 142 (0.47 g) in DCM (6 mL) was added oxalyl chloride (2 mL) followed by DEF (2 drops). The reaction was stirred for 22 h and was evaporated in vacuo to give (1R,2S,5S)-2-(chlorocarbonyl)-5-methylcyclohexyl acetate.

To a stirred solution of Intermediate 143 (0.20 g) and triphenylphosphine (0.20 g) in dry DCM (5 mL) was added (1R,2S,5S)-2-(chlorocarbonyl)-5-methylcyclohexyl acetate (0.17 g, prepared above) and the reaction was stirred at 45° C. for 2 h. The solvent was evaporated in vacuo and the residue was dissolved in DCE (4 mL). The mixture was heated at 87° C. for 3 days. The reaction was allowed to cool and was evaporated in vacuo. The crude material was purified by ISCO Companion silica chromatography, eluting with a gradient 0-100% EtOAc in cyclohexane to give the title compound.

¹H NMR (CDCl₃) δ 8.71 (1H, dd), 8.49 (1H, dd), 8.08 (2H, d), 7.80 (2H, d), 7.04 (1H, s), 6.84 (1H, dd), 5.21 (1H, d), 4.91 (1H, quintet), 3.87 (3H, s), 2.37 (1H, dt), 2.10 (3H, s), 1.79-1.55 (4H, m), 1.19 (3H, d), 1.01-0.95 (1H, m), 0.92 (3H, d), 0.87-0.81 (2H, m), 0.78 (3H, d), one aromatic proton not seen (obscured by triphenyphosphine impurity peaks, 7.73-7.43).

### Intermediate 145

### Methyl 3-{ethyl[(trans-4-methylcyclohexyl)carbonyl]amino}-5-iodo-2-thiophene carboxylate

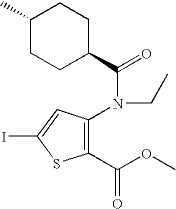

A solution of LDA (1.8M solution in THF/heptane/ethyl benzene, 6.1 mL) was cooled to −76° C. under an atmosphere of nitrogen. A solution of Intermediate 106 (1.14 g) in dry THF (12 mL) was added dropwise over 30 mins, keeping the internal temperature below −70° C. The reaction mixture was then stirred for 2 h. Iodine (1.87 g) in dry THF (12 mL) was then added dropwise over 30 mins keeping the internal temperature below −70° C. The reaction mixture was stirred for 1 h before being slowly quenched with saturated ammonium chloride solution (2 mL) and aqueous sodium thiosulphate solution (1.0 g in 12 mL of water). The reaction was allowed to warm to room temperature and was diluted with EtOAc and water. The aqueous phase was separated off and was extracted with EtOAc (×2). The organic phases were combined and were washed with water, dried over sodium sulphate and evaporated in vacuo. The crude material was purified using a silica Biotage cartridge, eluting with 20% EtOAc in cyclohexane to give the title compound.

MS calcd for (C₁₆H₂₂INO₃S+H)⁺: 436

MS found (electrospray): (M+H)⁺=436

### Intermediate 146

### Methyl 3-{ethyl[(trans-4-methylcyclohexyl)carbonyl]amino}-5-(4-pyrazolo[1,5-a]pyrimidin-2-ylphenyl)-2-thiophenecarboxylate

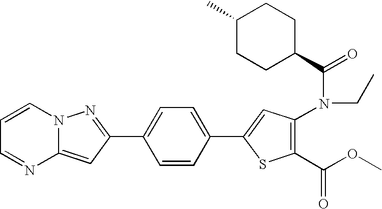

A mixture of Intermediate 145 (0.53 g), 2-[4-(4,4,5,5-tetramethyl-1,3,2-dioxaborolan-2-yl)phenyl]pyrazolo[1,5-a]pyrimidine (0.58 g, a synthesis of which is described above as Intermediate 10), 2N sodium carbonate solution (3.3 mL) and tetrakis(triphenylphosphine)palladium (0) (200 mg) in DMF (12 mL) was heated at 100° C. under nitrogen for 3 h. The reaction was evaporated in vacuo and the residue was partitioned between EtOAc and water. The organics were separated, washed with water (×2) and dried over sodium sulphate. The organics were evaporated in vacuo and the crude material was purified by ISCO Companion silica chromatography, eluting with a gradient 0-65% EtOAc in cyclohexane to give the title compound.

MS calcd for (C₂₈H₃₀N₄O₃S+H)⁺: 503

MS found (electrospray): (M+H)⁺=503

### Intermediate 147

### Methyl 4-[[(trans-4-methylcyclohexyl)carbonyl](1-methylethyl)amino]-5′-pyrazolo[1,5-a]pyrimidin-2-yl-2,2′-bithiophene-5-carboxylate

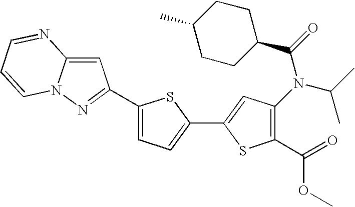

Tetrakis(triphenylphosphine)palladium (0) (353 mg) was added to a mixture of Intermediate 135 (2 g) {4-[[(trans-4-methylcyclohexyl)carbonyl](1-methylethyl)amino]-5-[(methyloxy)carbonyl]-2-thienyl}boronic acid (2.69 g, a synthesis of which is described above as Intermediate 64) and cesium fluoride (2.78 g) in DME (60 mL) and water (20 mL). The mixture was heated at 90° C. under nitrogen for 3 h. The reaction was allowed to cool and was poured into 8% sodium bicarbonate solution (100 mL). DCM (200 mL) was added and the layers were separated. The aqueous was extracted further with DCM (2×100 mL) then the combined organics were dried using a hydrophobic frit and evaporated. The crude material was purified by ISCO Companion silica chromatography, eluting with a gradient 0-100% EtOAc in cyclohexane to give the title compound.

MS calcd for (C₂₇H₃₀N₄O₃S₂+H)⁺: 523

MS found (electrospray): (M+H)⁺=523

### Intermediate 148

### Methyl 5′-iodo-4-[[(trans-4-methylcyclohexyl)carbonyl](1-methylethyl)amino]-2,2′-bithiophene-5-carboxylate

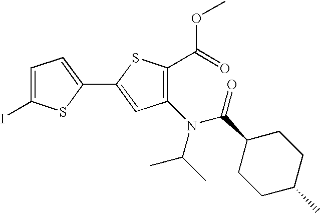

A mixture of 2,5-diiodothiophene (1.8 g), {4-[[(trans-4-methylcyclohexyl)carbonyl](1-methylethyl)amino]-5-[(methyloxy)carbonyl]-2-thienyl}boronic acid (1 g, a synthesis of which is described above as Intermediate 64), cesium fluoride (2.4 g) and tetrakis (triphenylphosphine)palladium (0) (376 mg) were dissolved in DME/water (1:1, 24 mL) and the reaction was heated at 90° C. for 2 h. The mixture was partitioned between EtOAc and water. The aqueous was extracted with EtOAc (×2) and the combined organics were dried using a hydrophobic frit and were evaporated in vacuo. The crude material was purified by ISCO Companion silica chromatography, eluting with a gradient 5-50% EtOAc in cyclohexane to give the title compound.

MS calcd for (C₂₁H₂₆INO₃S₂+H)⁺: 532

MS found (electrospray): (M+H)⁺=532

### Intermediate 149

### Methyl 4-[[(trans-4-methylcyclohexyl)carbonyl](1-methylethyl)amino]-5′-[(trimethylsilyl)ethynyl]-2,2′-bithiophene-5-carboxylate

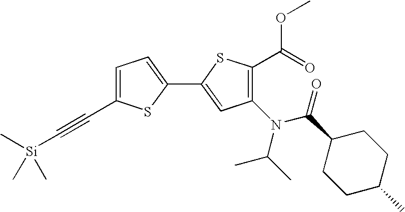

A mixture of methyl 5′-iodo-4-[[(trans-4-methylcyclohexyl)carbonyl](1-methylethyl)amino]-2,2′-bithiophene-5-carboxylate (570 mg, a synthesis of which is described above as Intermediate 148), trimethylsilyacetylene (0.272 mL), triethylamine (354 mg), dichlorobis(triphenylphosphine)palladium (II) (35 mg) and copper (I) iodide (9.5 mg) in THF (5 mL) was stirred at room temperature for 1 h under nitrogen. The reaction was diluted with DCM and was dried using a hydrophobic frit. The organics were evaporated in vacuo and the crude material was purified by ISCO Companion silica chromatography, eluting with a gradient 5-50% EtOAc in cyclohexane to give the title compound.

MS calcd for (C₂₆H₃₅NO₃S₂Si+H)⁺: 502

MS found (electrospray): (M+H)⁺=502

### Intermediate 150

### Ethyl 5′-ethynyl-4-[[(trans-4-methylcyclohexyl)carbonyl](1-methylethyl)amino]-2,2′-bithiophene-5-carboxylate

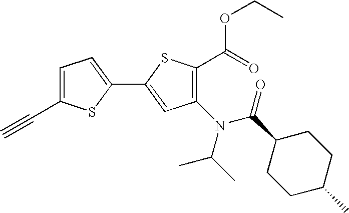

Intermediate 149 (304 mg) was dissolved in ethanol (6 mL) and potassium carbonate (42 mg) was added. The reaction mixture was stirred at room temperature for 18 h, and was partitioned between saturated sodium bicarbonate solution and DCM. The organics were separated using a hydrophobic frit and were evaporated in vacuo to give the title compound.

MS calcd for (C₂₄H₂₉NO₃S₂+H)⁺: 444

MS found (electrospray): (M+H)⁺=444

### Intermediate 151

### Ethyl 5′-furo[3,2-b]pyridin-2-yl-4-[[(trans-4-methylcyclohexyl)carbonyl](1-methylethyl)amino]-2,2′-bithiophene-5-carboxylate

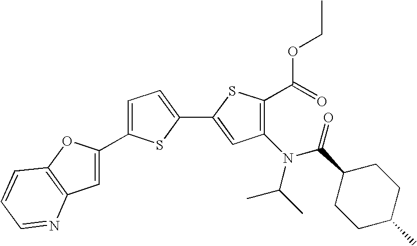

Intermediate 150 (250 mg), 2-iodo-3-pyridinol (125 mg), copper (I) iodide (10.7 mg) and dichlorobis(triphenylphosphine)palladium (II) (39 mg) were dissolved in triethylamine (3.5 mL) and were heated in a Reacti-vial at 70° C. overnight. The reaction was partitioned between saturated sodium bicarbonate solution and DCM. The organics were dried using a hydrophobic frit and were evaporated in vacuo. The crude material was purified by ISCO Companion silica chromatography, eluting with a gradient 5-100% EtOAc in cyclohexane to give the title compound.

MS calcd for (C₂₉H₃₂N₂O₄S₂+H)⁺: 537

MS found (electrospray): (M+H)⁺=537

### Intermediate 152

### 1,1-Dimethylethyl 6-bromo-1H-indole-1-carboxylate

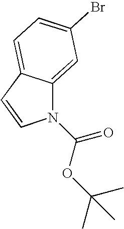

6-Bromo-1H-indole (1.01 g) and di-tert-butyl dicarbonate (1.34 g) were dissolved in MeCN (50 mL) and DMAP (127 mg) was added. The mixture was stirred for 4 h, then a further portion of di-tert-butyl dicarbonate (218 mg) was added. The mixture was stirred at room temperature for 16 h. The mixture was partitioned between DCM and water, and the organics were separated and evaporated in vacuo. The residue was partitioned between DCM and 2M HCl and the organics were separated and evaporated in vacuo to give the title compound.

¹H NMR (CDCl₃) δ 8.36 (1H, s), 7.55 (1H, d), 7.41 (1H, d), 7.34 (1H, dd), 6.53 (1H, d), 1.67 (9H, s).

### Intermediate 153

### (4-{4-[[(trans-4-Methylcyclohexyl)carbonyl](1-methylethyl)amino]-5-[(methyloxy)carbonyl]-2-thienyl}phenyl)boronic acid

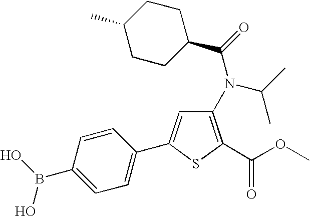

A mixture of tetrakis(triphenylphosphine)palladium (0) (42.4 mg), methyl 5-iodo-3-[[(trans-4-methylcyclohexyl)carbonyl](1-methylethyl)amino]-2-thiophenecarboxylate (330 mg, a synthesis of which is described above as Intermediate 5, method B) and benzene-1,4-diyldiboronic acid (608 mg) in 1,4-dioxane (3 mL) and 2N sodium carbonate solution (0.5 mL) was stirred at room temperature for 1 minute. The reaction mixture was heated at 100° C. in a microwave for 10 mins. The mixture was allowed to cool. The above reaction was repeated twice, and the 3 cooled reactions mixtures were combined and evaporated in vacuo. The residue was partitioned between DCM (50 mL) and water (20 mL), and the organics were separated and filtered. The aqueous layer was extracted with DCM (50 mL), then the combined organics were dried using a hydrophobic frit and evaporated in vacuo. The crude material was purified by ISCO Companion silica chromatography to give the title compound. TLC (Silica gel 60 F₂₅₄) eluted with 1:1 EtOAc/cyclohexane, title compound R_{f}=0.30.

### Intermediate 154

### Methyl 5-[4-(1H-indol-5-yl)phenyl]-3-[[(trans-4-methylcyclohexyl)carbonyl](1-methylethyl)amino]-2-thiophenecarboxylate

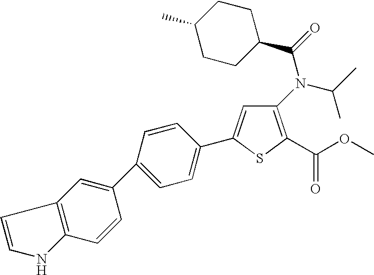

A mixture of (4-{4-[[(trans-4-methylcyclohexyl)carbonyl](1-methylethyl)amino]-5-[(methyloxy)carbonyl]-2-thienyl}phenyl)boronic acid (100 mg, a synthesis of which is described above as Intermediate 153), 5-bromo-1H-indole (66.4 mg) and tetrakis(triphenylphosphine)palladium (0) (25.8 mg) in 1,4-dioxane (9 mL) and 2N sodium carbonate solution (3 mL) was heated at 100° C. under nitrogen for 2 h. The reaction was partitioned between DCM and water and the organics were separated and purified by MDAP HPLC to give the title compound.

MS calcd for (C₃₁H₃₄N₂O₃S+H)⁺: 516

MS found (electrospray): (M+H)⁺=516

### Intermediate 155

### Methyl 5-[4-(1H-indol-6-yl)phenyl]-3-[[(trans-4-methylcyclohexyl)carbonyl](1-methylethyl)amino]-2-thiophenecarboxylate

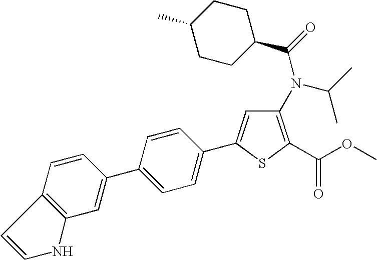

A mixture of Intermediate 153 (150 mg), Intermediate 152 (134.2 mg) and tetrakis(triphenylphosphine)palladium (0) (19.6 mg) in 1,4-dioxane (3 mL) and 2N sodium carbonate solution (0.5 mL) was stirred for 1 minute and was then heated at 100° C. in a microwave for 10 mins. The reaction was left to stand for 3 days and was then heated at 100° C. in a microwave for 10 mins. The reaction was diluted with DCM (50 mL) and was washed with sodium carbonate solution (50 mL). The organics were separated and evaporated in vacuo. The crude material was purified by ISCO Companion silica chromatography, eluting with a gradient 6:1 cyclohexane/EtOAc. The material was purified further by MDAP HPLC to give the title compound.

¹H NMR (CD₃OD) δ 10.58 (1H, br), 7.82-7.73 (4H, m), 7.69 (1H, d), 7.64-7.60 (1H, m), 7.40 (1H, s), 7.35 (1H, dd), 7.29-7.27 (1H, m), 6.48-6.45 (1H, m), 4.85 (1H, m, partially obscured by water peak), 3.84 (3H, s), 2.09 (1H, tt), 1.76-1.52 (5H, m), 1.46-1.25 (2H, m), 1.23 (3H, d), 0.97 (3H, d), 0.78 (3H, d), 0.75-0.55 (2H, m).

### Intermediate 156

### Methyl 3-[[(4-methylcyclohexyl)carbonyl](1-methylethyl)amino]-5-(4-[1,3]oxazolo[4,5-b]pyridin-2-ylphenyl)-2-thiophenecarboxylate

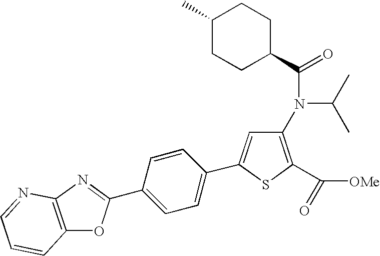

A mixture of {4-[[(trans-4-methylcyclohexyl)carbonyl](1-methylethyl)amino]-5-[(methyloxy)carbonyl]-2-thienyl}boronic acid (9.35 g, 25.5 mmol, a synthesis of which is described as Intermediate 64), 2-(4-bromophenyl)[1,3]oxazolo[4,5-b]pyridine (7.01 g, 25.5 mmol, a synthesis of which is described as Intermediate 36), cesium fluoride (19.35 g, 127 mmol), DME (50 mL), water (50 mL) and Pd(PPh₃)₄ (3.92 g, 2.55 mmol) was heated under reflux with stirring for 2 h. The mixture was allowed to cool and was partitioned between EtOAc and water. The aqueous phase was separated and washed with EtOAc (3×). The combined organic fractions were washed with water and brine, dried (MgSO₄) and evaporated in vacuo. The residue was purified using ISCO silica chromatography eluting with a gradient of EtOAc in cyclohexane (0-20%) to give the title compound.

MS calcd for (C₂₉H₃₁N₃O₄S+H)⁺: 518

MS found (electrospray): (M+H)⁺=518

### Intermediate 157

### {4-((1-Methylethyl){[trans-4-(trifluoromethyl)cyclohexyl]carbonyl}amino)-5-[(methyloxy)carbonyl]-2-thienyl}boronic acid

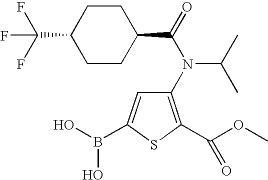

Methyl 3-((1-methylethyl){[trans-4-(trifluoromethyl)cyclohexyl]carbonyl}amino)-2-thiophene carboxylate (1 g, a synthesis of which is described as Intermediate 75) in THF (5 mL) was added dropwise to LDA (4.41 mL, 1.8M solution in THF/heptane/ethylbenzene) in THF (5 mL) at −78° C. under nitrogen (maintaining the internal temperature at −78° C.). This was stirred at −78° C. for 45 mins. Trimethylborate (887 uL) in THF (5 mL) was added dropwise, maintaining the internal temperature at −78° C. After ca 30 mins, 2N hydrochloric acid (15 mL) was added dropwise to quench the reaction and the reaction was allowed to warm to room temperature over ca 1 h. EtOAc (40 mL) was added and the layers separated. The aqueous layer was extracted further with EtOAc (2×10 mL). The combined organic extracts were dried (Na₂SO₄) and evaporated, and triturated with diethyl ether to give the title compound.

MS calcd for (C₁₇H₂₃BF₃NO₅S+H)⁺: 422

MS found (electrospray): (M+H)⁺=422

### Intermediate 158

### 2-(5-Bromo-2-pyridinyl)[1,3]oxazolo[4,5-b]pyridine

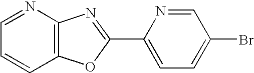

A mixture of 2-amino-3-pyridinol (1.0 g), 5-bromo-2-pyridinecarboxylic acid (2.76 g) and polyphosphoric acid (21 g) was heated at 190° C. for 40 mins. The mixture was cooled and basified with sodium carbonate solution (300 mL) and was extracted with DCM (4×100 mL). The combined organics were dried using a hydrophobic frit and were evaporated in vacuo. The crude material was purified using an NH₂ SPE cartridge, eluting with MeOH to give the title compound.

MS calcd for (C₁₁H₆BrN₃O+H)⁺: 276/278

MS found (electrospray): (M+H)⁺=276/278

### Intermediate 159

### Methyl 3-[[(trans-4-methylcyclohexyl)carbonyl](1-methylethyl)amino]-5-(6-[1,3]oxazolo[4,5-b]pyridin-2-yl-3-pyridinyl)-2-thiophenecarboxylate

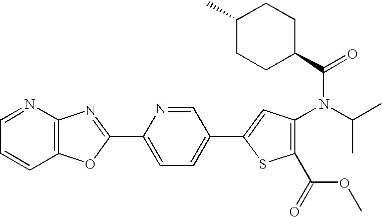

A mixture of Intermediate 64 (102 mg), Intermediate 158 (64 mg), tetrakis-(triphenylphosphine)palladium (0) (50 mg) and sodium carbonate (73 mg) in 1,4-dioxane (3 mL) was heated in a microwave at 80° C. for 15 mins, 80° C. for 15 mins and 100° C. for 15 mins. A further portion of Intermediate 64 (44 mg) was added and the reaction was heated in a microwave at 100° C. for 20 mins and 120° C. for 30 mins. Water (0.2 mL) was added and the reaction was heated in a microwave at 110° C. for 20 mins. The reaction was evaporated in vacuo and the crude material was purified by silica SPE, eluting with cyclohexane, followed by a gradient of DCM to EtOAc. The material was purified further using a SCX-2 ion exchange cartridge, eluting with MeOH (×2), followed by 10% triethylamine in MeOH (×3) to give the title compound.

MS calcd for (C₂₅H₃₀N₄O₄S+H)⁺: 519

MS found (electrospray): (M+H)⁺=519

### Intermediate 160

### trans-4-Methyl-cyclohexanecarbonyl chloride

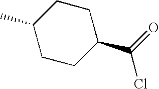

A solution of trans-4-methyl-cyclohexanecarboxylic acid (155.6 g) in DCM (1000 mL) was treated with thionyl chloride (192 mL) at 20-25° C. over 2-3 h. The mixture was warmed to slight reflux for 1 h and stirred overnight at room temperature. The mixture was evaporated in vacuo at 25-30° C., diluted with DCM (200 mL) and reconcentrated to give the title compound.

GC (column HP35, 30 mm×0.25 mm×0.25 um, detection temp 250° C., 20 mins run, retention time 1.89 mins)

### Example 1

### 5-(4-Furo[3,2-b]pyridin-2-ylphenyl)-3-[[(trans-4-methylcyclohexyl)carbonyl](1-methylethyl)amino]-2-thiophenecarboxylic acid

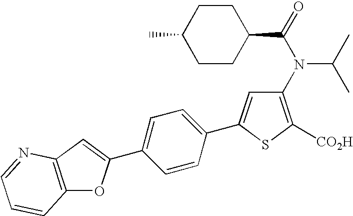

Intermediate 8 (35 mg) and sodium hydroxide solution (2M, 0.2 mL) in THF (0.2 mL) and MeOH (0.2 mL) were stirred at room temperature for 22 h. The mixture was partitioned between 2M HCl (5 mL) and DCM (5 mL). The aqueous fraction was extracted further with DCM (5 mL) and the combined organics evaporated. This was purified by MDAP to give the title compound.

MS calcd for (C₂₅H₃₀N₂O₄S+H)⁺: 503

MS found (electrospray): (M+H)⁺=503

¹H NMR (CD₃OD): δ8.48 (1H, d), 8.1 (2H, d), 8.01 (1H, d), 7.92 (2H, d), 7.48 (2H, d), 7.38 (1H, m), 2.12 (1H, m), 1.9-1.2 (8H, m), 1.25 (3H, d), 1.05 (3H, d), 0.79 (3H, d), 0.76-0.6 (2H, m), carboxylic acid proton not seen.

### Example 2

### 3-[[(trans-4-Methylcyclohexyl)carbonyl](1-methylethyl)amino]-5-(4-pyrazolo[1,5-a]pyrimidin-2-ylphenyl)-2-thiophenecarboxylic acid

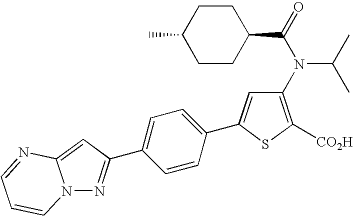

Intermediate 11 (94 mg) and sodium hydroxide solution (2M, 0.5 mL) in THF (0.5 mL) and MeOH (0.5 mL) were stirred at room temperature for 24 h. The mixture was evaporated and partitioned between 2M HCl (10 mL) and DCM (10 mL). The aqueous was extracted further with DCM (10 mL) and the combined organics evaporated. This was purified by SPE chromatography, eluting with cyclohexane to cyclohexane/EtOAc (2:1) to (1:1) to (1:2) to EtOAc; EtOAc/MeCN (1:1), MeCN, MeCN/acetone (1:1) to acetone to acetone/MeOH (1:1) to MeOH. This was purified further using MDAP and freeze dried from dioxane. Further purification using an NH₂ ion exchange SPE cartridge gave the title compound.

MS calcd for (C₂₈H₃₀N₄O₃S+H)⁺: 503

MS found (electrospray): (M+H)⁺=503

¹H NMR (CD₃OD): δ 8.94 (1H, d), 8.51 (1H, dd), 8.14 (2H, d), 7.88 (2H, d), 7.46 (1H, s), 7.12 (1H, s), 7.05 (1H, m), 2.12 (1H, m), 1.8-1.25 (8H, m), 1.24 (3H, d), 1.01 (3H, d), 0.79 (3H, d), 0.76-0.6 (2H, m), carboxylic acid proton not seen.

### Alternative preparation of Example 2 (Method B)

### 3-[[(trans-4-Methylcyclohexyl)carbonyl](1-methylethyl)amino]-5-(4-pyrazolo[1,5-a]pyrimidin-2-ylphenyl)-2-thiophenecarboxylic acid

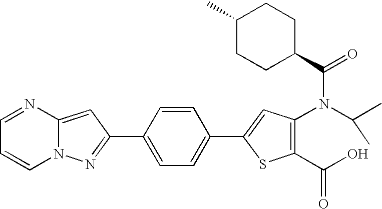

To methyl 3-[[(trans-4-methylcyclohexyl)carbonyl](1-methylethyl)amino]-5-(4-pyrazolo[1,5-a]pyrimidin-2-ylphenyl)-2-thiophenecarboxylate (14.05 g, a synthesis of which is described above as Intermediate 11, method B) was added THF (80 mL), MeOH (80 mL) and 2N sodium hydroxide solution (80 mL), and the mixture was stirred at room temperature overnight. The reaction mixture was acidified with 2N HCl (90 mL) and was concentrated in vacuo until nearly all the solvent had evaporated. The mixture was partitioned between water and DCM and the solid was filtered off. The filtrate was separated by hydrophobic frit and the organics were combined with the solid obtained from the filtration step. The crude material was purified by reverse phase ISCO Companion silica chromatography, using a C18 cartridge, eluting with a gradient 5% MeCN (0.05% formic acid)/water (0.1% formic acid) to 100% MeCN (0.05% formic acid). The product containing fractions were concentrated in vacuo until a solid precipitated from the solution. The solid was collected by filtration, dried in a vacuum oven at 50° C. for 2 days, then in a vacuum over P₂O₅ at room temperature overnight to give the title compound.

MS calcd for (C₂₈H₃₀N₄O₃S+H)⁺: 503

MS found (electrospray): (M+H)⁺=503

¹H NMR (d₆-DMSO) δ 13.35 (1H, br), 9.16 (1H, dt), 8.56 (1H, dd), 8.15 (2H, dd), 7.95 (2H, d), 7.65 (1H, s), 7.35 (1H, d), 7.07 (1H, dd), 4.76 (1H, quintet), 1.99 (1H, tt), 1.69-1.42 (5H, m), 1.31-1.18 (2H, m), 1.15 (3H, d), 0.91 (3H, d), 0.74 (3H, d), 0.72-0.48 (2H, m).

### Example 3

### 5-(4-Imidazo[1,2-a]pyridin-2-ylphenyl)-3-[[(trans-4-methylcyclohexyl)carbonyl](1-methylethyl)amino]-2-thiophenecarboxylic acid

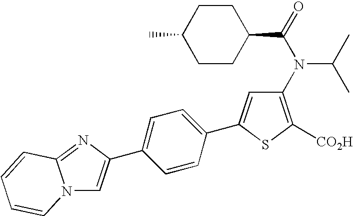

Intermediate 13 (36 mg) and sodium hydroxide solution (2M, 0.2 mL) in THF (0.2 mL) and MeOH (0.2 mL) were stirred at room temperature for 4 h. The mixture was evaporated and then partitioned between 2M HCl (5 mL) and DCM (5 mL). A solid formed in the aqueous phase which was collected, dissolved in methanol and co-evaporated with diethyl ether. Freeze-drying from dioxane gave the title compound.

MS calcd for (C₂₉H₃₁N₃O₃S+H)⁺: 502

MS found (electrospray): (M+H)⁺=502

¹H NMR (CD₃OD): δ 8.81 (1H, d), 8.66 (1H, s), 8.05-7.9 (6H, m), 7.6-7.48 (2H, m), 2.12 (1H, m), 1.8-1.25 (8H, m), 1.24 (3H, d), 1.0 (3H, d), 0.79 (3H, d), 0.76-0.6 (2H, m), carboxylic acid proton not seen.

### Example 4

### 5-(4-Imidazo[2,1-b][1,3]thiazol-6-ylphenyl)-3-[[(trans-4-methylcyclohexyl)carbonyl](1-methylethyl)amino]-2-thiophenecarboxylic acid

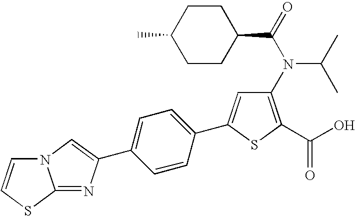

Intermediate 25 (300 mg) was dissolved in THF (5 mL)/EtOH (5 mL). To this solution lithium hydroxide solution (2N, 5 mL) was added and the mixture stirred at room temperature for 70 h. The ethanol and THF were evaporated under vacuum. Water (40 mL) was added, the mixture acidified with 2N HCl to pH 1.0 and extracted with DCM). The organic fractions were passed through a hydrophobic frit and freeze dried (from dioxane) to give the title compound.

MS calcd for (C₂₇H₂₉N₃O₃S₂+H)⁺: 508

MS found (electrospray): (M+H)⁺=508

¹H NMR (MeOD): [symbol] 8.15 (1H, s), 7.90 (½ AA′ BB′, 2H), 7.78 (3H, m), 7.35 (1H, s), 7.15 (1H, d), ˜4.88 (1H, partially hidden by solvent peak), 2.15 (1H, m), 1.25-1.9 (7H, m), 1.23 (3H, d), 1.00 (3H, d), 0.76, (3H, d), 0.55-0.73 (m, 2H), carboxylic acid proton not seen.

### Example 5

### 5-[4-(7-Amino-5-methylpyrazolo[1,5-a]pyrimidin-2-yl)phenyl]-3-[[(trans-4-methylcyclohexyl)carbonyl](1-methylethyl)amino]-2-thiophenecarboxylic acid

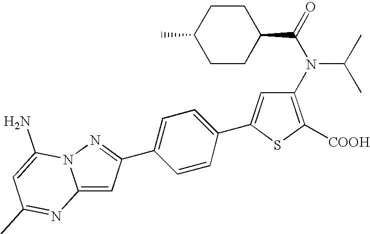

Intermediate 28 (300 mg) was dissolved in THF (1.5 mL) and methanol (1.5 mL). Sodium hydroxide solution (2N, 1.5 mL) was added and the mixture stirred at room temperature for 72 h. The methanol and THF were evaporated under vacuum. The residue was purified by ISCO companion C18 chromatography eluted with water (0.1% formic acid) then a gradient of 5-100% acetonitrile (containing 0.05% formic acid) to give the title compound.

MS calcd for (C₂₉H₃₃N₅O₃S+H)⁺: 532

MS found (electrospray): (M+H)⁺=532

¹H NMR (MeOD): δ 8.35 (2H, br) 8.1 (d, ½ AA′ BB′), 7.8 (2H, ½ AA′ BB′), 7.35 (1H, s), 6.7 (1H, d), 6.05 (1H, s), ˜4.88 (1H, partially hidden by solvent peak), 2.43 (3H, s), 2.2 (1H, m), 1.85 (1 h, m), 1.26-1.75 (6H, m), 1.25 (3H, d), 1.00 (3H, d), 0.78, (3H, d), 0.76-0.56 (2H, m) 0.55-0.75 (m, 2H), carboxylic acid proton not seen.

### Example 6

### 3-[[(trans-4-Methylcyclohexyl)carbonyl](1-methylethyl)amino]-5[4-(5-methylpyrazolo[1,5-a]pyrimidin-2-yl)phenyl]-2-thiophenecarboxylic acid

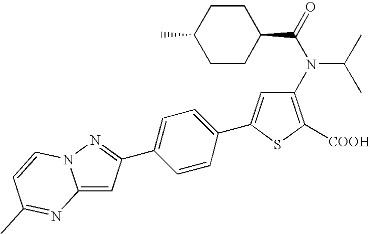

Intermediate 31 (458 mg) was dissolved in THF (1.5 mL) and methanol (1.5 mL). 2N Sodium hydroxide solution (2N, 1.5 mL) was added and the mixture stirred at room temperature for 20 h. The methanol and THF were evaporated under vacuum. The residue was partitioned between DCM/1N HCl. The organic layer was passed through a hydrophobic frit and evaporated in vacuum. The residue was triturated with methanol and dried under vacuum to give the title compound.

MS calcd for (C₂₉H₃₂N₄O₃S+H)⁺: 517

MS found (electrospray): (M+H)⁺=517

¹H NMR (DMSO): [symbol] 9.0 (1H, s), 8.12 (2H, ½ AA′ BB′), 7.92 (2H, ½ AA′ BB′), 7.65 (1H, s), 7.15 (1H, s), 6.95 (1H, d), 4.75 (1H, m), 2.54 (3H, s), 2.00 (1H, m), 1.18-1.7 (7H, m), 1.15 (3H, d), 0.9 (3H, d), 0.7, (3H, d), 0.7-0.45 (m, 2H), carboxylic acid proton not seen.

### Example 7

### 5-[4-(7-Aminopyrazolo[1,5-a]pyrimidin-2-yl)phenyl]-3-[[(trans-4-methylcyclohexyl)carbonyl](1-methylethyl)amino]-2-thiophenecarboxylic acid

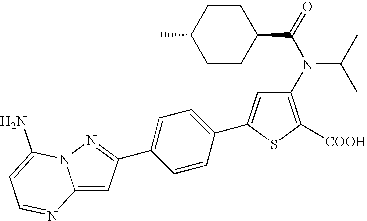

Intermediate 34 (381 mg) was dissolved in THF (1.5 mL) and methanol (1.5 mL). 2N Sodium hydroxide solution (1.5 mL) was added and the mixture stirred at room temperature for 72 h. The methanol and THF were evaporated under vacuum. The residue was dissolved in methanol. Product was then applied to an aminopropyl SPE cartridge, eluted with methanol, followed by 10% HCl solution in methanol. Fractions were then evaporated to dryness and then dissolved in 1:1 MeOH/DMSO and filtered. The filtrate was purified by ISCO companion C18 chromatography eluted with water (0.1% formic acid) then a gradient of 5-100% acetonitrile (containing 0.05% formic acid) to give the title compound.

MS calcd for (C₂₈H₃₁N₅O₃S+H)⁺: 518

MS found (electrospray): (M+H)⁺=518

¹H NMR (MeOD): [symbol] 8.15 (2H, ½ AA′ BB′), 8.07 (1H, d), 7.85 (2H, ½ AA′ BB′), 7.4, (1H, s), 6.83, (s, 1H), 6.15 (1H, d), (˜4.88 (1H, partially hidden by solvent peak), 2.15 (1H, m), 1.25-1.9 (7H, m), 1.25 (3H, d), 1.03 (3H, d), 0.76, (3H, d), 0.55-0.73 (m, 2H), carboxylic acid and amino protons not seen.

### Example 8

### 3-[[(4-Methylcyclohexyl)carbonyl](1-methylethyl)amino]-5-(4-[1,3]oxazolo[4,5-b]pyridin-2-ylphenyl)-2-thiophenecarboxylic acid

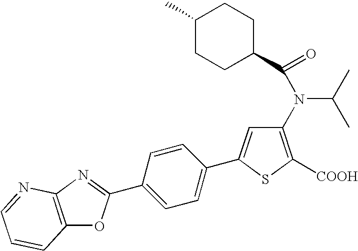

To Intermediate 35 (540 mg) in dry DMF (10 ml) was added Intermediate 37 (522 mg), sodium carbonate (2N, 3.12 mL), and Pd(PPh₃)₄ (202 mg), and the reaction mixture was then heated to 100° C. under nitrogen and with stirring for 1 h. The reaction mixture was allowed to cool, and the DMF was removed by evaporation. The residue was partitioned between water and diethyl ether. The organic layer was separated and the aqueous acidified with 2N HCl to pH=1. The aqueous layer was extracted with EtOAc, separated, dried over magnesium sulphate, filtered and evaporated to an oil. This was then purified by ISCO companion silica chromatography eluting with a gradient of EtOAc/cyclohexane (0% to 100%). The product was then further purified by ISCO companion silica chromatography eluting with a gradient of methanol/DCM (0% to 100%). Further purification by SPE silica chromatography with a gradient of methanol/DCM (0% to 90%), and evaporating the purest fractions gave a solid. This was partitioned between DCM and water, the organic layer removed, dried over magnesium sulphate, filtered and evaporated to give a solid which was placed on a NH₂ propyl SPE column and eluted with methanol, followed by 10% acetic acid in methanol to give a solid after removal of solvent. This product was then repurified by ISCO companion silica chromatography eluting with a gradient of EtOAc/cyclohexane (0% to 100%) to give the title compound.

MS calcd for (C₂₈H₂₉N₃O₄S+H)⁺: 504

MS found (electrospray): (M+H)⁺=504

¹H NMR (MeOD): δ 8.85 (1H, brd), 8.40 (2H, ½ AA′ BB′), 8.15 (1H, d), 8.00 (2H, ½ AA′ BB′), 7.61 (1H, s), 7.45 (1H, dd), ˜4.88 (1H, partially hidden by solvent peak), 2.1 (1H, tt), 1.55-1.85 (5H, m), 1.20-1.50 (2H, m), 1.02 (3H, d), 1.00, (3H, d), 0.8 (3H, d), 0.55-0.80 (2H, m), carboxylic acid proton not seen.

### Alternative Preparation of Example 8 (Method B)

### 3-[[(4-Methylcyclohexyl)carbonyl](1-methylethyl)amino]-5-(4-[1,3]oxazolo[4,5-b]pyridin-2-ylphenyl)-2-thiophenecarboxylic acid

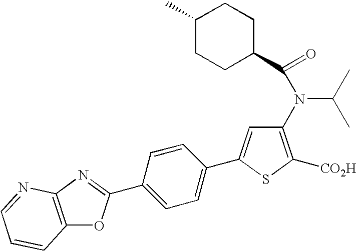

To a solution of Intermediate 156 (2.74 g, 5.3 mmol, a synthesis of which is describe as Intermediate 156) in pyridine (40 mL) was added lithium iodide (3.55 g, 5 eq.) and the mixture was heated at 105° C. under nitrogen for 13 hours. The solvent was evaporated in vacuo, hydrochloric acid (2N) added and the mixture extracted with ethyl acetate (3×). The organic fractions were combined, dried (MgSO₄) and evaporated. The residue was purified using ISCO silica chromatography eluting with DCM followed by a gradient of DCM in MeOH (0-12%) to give the title compound.

MS calcd for (C₂₈H₂₉N₃O₄S+H)⁺: 504

MS found (electrospray): (M+H)⁺=504

### Example 9

### 5-(4-Furo[2,3-b]pyridin-5-ylphenyl)-3-[[(trans-4-methylcyclohexyl)carbonyl](1-methylethyl)amino]-2-thiophenecarboxylic acid

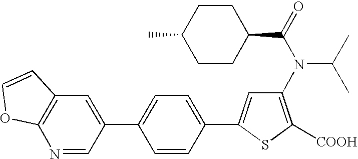

Intermediate 44 (152 mg) was dissolved in THF (1.5 mL) and methanol (1.5 mL). Lithium hydroxide solution (2N, 1.5 mL) was added and the mixture stirred at room temperature for 3 h. The methanol and THF were evaporated under vacuum, and the residue was partitioned between DCM and 2N HCl solution. The DCM layer was separated using a hydrophobic frit and freeze dried to give the title compound.

MS calcd for (C₂₉H₃₀N₂O₄S+H)⁺: 503

MS found (electrospray): (M+H)⁺=503

¹H NMR (MeOD): [symbol] 8.58 (1H, d), 8.38 (1H, d), 7.95 (1H, d), 7.85 (2H, ½ AA′ BB′), 7.78, (2H, ½ AA′ BB′), 7.28 (1H, s), 7.05 (1H, d), (˜4.88 (1H, partially hidden by solvent peak), 2.23 (1H tt), 1.9 (1H, d), 1.5-1.85 (4H, m) 1.25-1.43 (2H, m), 1.23 (3H, m), 1.02 (3H, d), 0.78, (3H, d), 0.55-0.76 (2H, m), carboxylic acid proton not seen.

### Example 10

### 5-[4-(5-Amino-1,3-benzoxazol-2-yl)phenyl]-3-[[(trans-4-methylcyclohexyl)carbonyl](1-methylethyl)amino]-2-thiophenecarboxylic acid

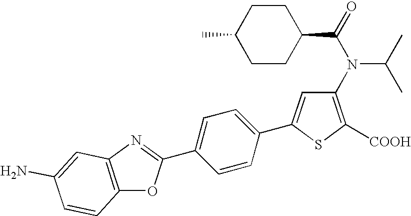

Intermediate 48 (365 mg) was dissolved in THF (1.9 mL) and methanol (1.9 mL). Sodium hydroxide solution (2N, 1.9 mL) was added and the mixture stirred at room temperature for 4 h. The methanol and THF were evaporated under vacuum. The residue was dissolved in water (25 mL), washed with ether and acidified with 2 N HCl. The aqueous layer was extracted with EtOAc. The EtOAc was washed with brine (50 mL) and organic layer separated and evaporated to give the title compound.

MS calcd for (C₂₉H₃₁N₃O₄S+H)⁺: 518

MS found (electrospray): (M+H)⁺=518

¹H NMR (MeOD): [symbol] 8.21 (2H, ½ AA′ BB′), 7.90 (2H, ½ AA′ BB′), 7.45 (1H, s), 7.39 (1H, d), 7.04 (1H, d), 6.83, (1H, dd), ˜4.88 (1H, partially hidden by solvent peak), 2.19 (1H, m), 1.85 (1H, m), 1.5-1.75 (4H, m), 1.25-1.42 (2H, m), 1.29 (3H, d), 1.03 (3H, d), 0.76, (3H, d), 0.55-0.73 (m, 2H), carboxylic acid and amino protons not seen.

### Example 11

### 3-[[(4-Methylcyclohexyl)carbonyl](1-methylethyl)amino]-5-(4-[1,3]oxazolo[5,4-b]pyridin-2-ylphenyl)-2-thiophenecarboxylic acid

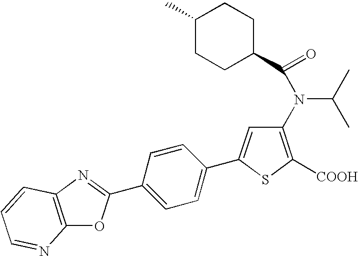

To Intermediate 35 (690 mg) in dry DMF (10 mL) was added Intermediate 51 (1.9 g), sodium carbonate solution (2 N, 4 mL), and Pd(PPh₃)₄ (259 mg), and the reaction mixture was then heated to 100° C. under nitrogen with stirring for 1 h. The reaction mixture was allowed to cool, and the DMF was removed by evaporation. The residue was then partitioned between water and EtOAc. The aqueous layer was acidified with 2N HCl to pH=2. The aqueous layer was extracted into EtOAc, washed with brine, dried over sodium sulphate, filtered and evaporated. This was then purified by ISCO companion silica chromatography eluting with a gradient of EtOAc/cyclohexane (0% to 60%), and then repurified by SPE chromatography eluting with EtOAc/cyclohexane 50:50, then ethyl acetate, then DCM, and finally DCM/methanol to give the title compound.

MS calcd for (C₂₈H₂₉N₃O₄S+H)⁺: 504

MS found (electrospray): (M+H)⁺=504

¹H NMR (MeOD): [symbol] 8.32 (1H, dd), 8.29, (2H, ½ AA′ BB′), 8.15 (1H, dd), 7.95 (2H, ½ AA′ BB′), 7.53 (1H, s), 7.46 (1H, dd), 7.52 (2H, s), 7.46, (1H, dd), ˜4.88 (1H, partially hidden by solvent peak), 2.18 (1H, tt), 1.85 (1H, bd), 1.73 (1H, bd), 1.5-1.68 (3H, m), 1.28 (1H, m), 1.25 (3H, d), 1.03 (3H, d), 0.77 (3H, d), 0.53-0.75 (2H, 2m), carboxylic acid proton not seen.

### Example 12

### 5-(4-Furo[3,2-c]pyridin-2-ylphenyl)-3-[[(trans-4-methylcyclohexyl)carbonyl](1-methylethyl)amino]-2-thiophenecarboxylic acid

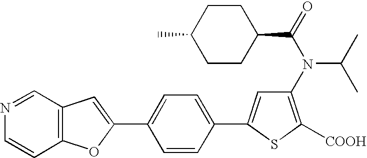

Intermediate 53 (494 mg) was dissolved in THF (5 mL) and methanol (5 mL). Lithium hydroxide solution (2N, 1.9 mL) was added and the mixture stirred at room temperature for 24 h. The methanol and THF were evaporated under vacuum, and the residue was dissolved in 2N sodium hydroxide (30 mL) and washed with ether. The aqueous was then acidified with 2N HCl, extracted with EtOAc, then chloroform, and the remaining insoluble material filtered off. The solid was dissolved in methanol and combined with the EtOAc and chloroform extracts and evaporated to a solid which was purified using an Oasis cartridge and eluted with water, methanol and THF. Pure fractions were evaporated under vacuum. This was dissolved in sodium hydroxide and extracted with ether. The aqueous layer was acidified with 2N HCl and extracted with EtOAc and chloroform. The aqueous layer was then filtered and organic extracts and solid combined and evaporated to give a solid which was recrystallised from hot isopropanol to give the title compound.

MS calcd for (C₂₉H₃₀N₂O₄S+H)⁺: 503

MS found (electrospray): (M+H)⁺=503

¹H NMR (DMSO): δ 13.38 (1H, brs), 9.0 (1H, brs), 8.5 (1H, brd), 8.06 (2H, ½ AA′ BB′), 7.98 (2H, ½ AA′ BB′), 7.75 (1H, d), 7.71 (1H, s), 7.69 (1H, s), 4.76 (1H, m), 1.98 (1H, tt), 1.4-1.70 (5H, m), 1.18-1.30 (2H, m), 1.15 (3H, d), 0.91 (3H, d), 0.74 (3H, d), 0.48-0.72 (m, 2H), carboxylic acid proton not seen.

### Example 13

### Sodium 5-(4-furo[3,2-c]pyridin-2-ylphenyl)-3-[[(trans-4-methylcyclohexyl)carbonyl](1-methylethyl)amino]-2-thiophenecarboxylate

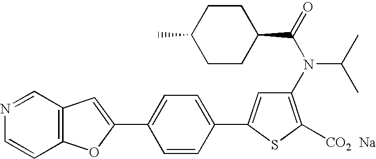

Example 12 was suspended in water (1 mL) and treated with 2N Sodium hydroxide solution (63 μl). Dioxane was then added and the solution freeze dried to give the title compound.

MS calcd for (C₂₉H₃₀N₂O₄S+H)⁺: 503

MS found (electrospray): (M+H)⁺=503

¹H NMR (d₆-DMSO): δ 8.97 (1H, brs), 8.49 (1H, brd), 8.00 (2H, d), 7.98 (2H, d), 7.84 (2H, d), 7.73 (1H, brd), 7.63 (1H, s), 4.69 (1H, m), 2.13 (1H, tt), 1.81 (1H, bd), 1.29-1.65 (4H, m), 1.14-1.28 (2H, m), 1.10 (3H, d), 0.915 (3H, d), 0.75 (3H, d), 0.37-0.69 (2H, m).

### Example 14

### 5-(4-Imidazo[1,2-a]pyridin-2-ylphenyl)-3-((1-methylethyl){[trans-4-(trifluoromethyl)cyclohexyl]carbonyl}amino)-2-thiophenecarboxylic acid

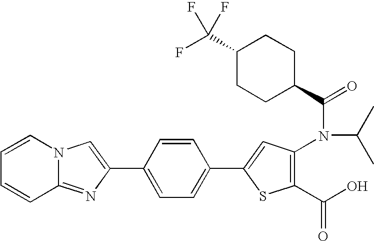

Intermediate 74 (97 mg) was dissolved in THF (1.5 mL) and ethanol (1.5 mL). The mixture was treated with 2N sodium hydroxide solution (1.0 mL) and was stirred overnight at room temperature. DCM (12 mL) was added followed by 2N HCl solution (5 mL) and the mixture was stirred vigorously for 10 mins. The organics were separated using a hydrophobic frit and were evaporated in vacuo. The crude material was purified by MDAP HPLC followed by NH₂ SPE cartridge eluting with 10% AcOH in MeOH to give the title compound.

MS calcd for (C₂₉H₂₈F₃N₃O₃S+H)⁺: 556

MS found (electrospray): (M+H)⁺=556

¹H NMR (d₆-DMSO) δ 8.52 (1H, d), 8.44 (1H, s), 8.00 (2H, d), 7.77 (2H, d), 7.59 (2H, d), 7.33-7.22 (2H, m), 6.90 (1H, t), 4.75-4.67 (1H, m), 2.01-2.13 (2H, m), 2.00-1.92 (1H, m), 1.83-1.65 (3H, m), 1.54-1.39 (1H, m), 1.35-1.21 (1H, m), 1.12 (3H, d), 0.93 (3H, d), 0.90-0.78 (1H, m), carboxylic acid proton not seen.

### Example 15

### 3-((1-Methylethyl){[trans-4-(trifluoromethyl)cyclohexyl]carbonyl}-amino)-5-(4-pyrazolo[1,5-a]pyrimidin-2-ylphenyl)-2-thiophenecarboxylic acid

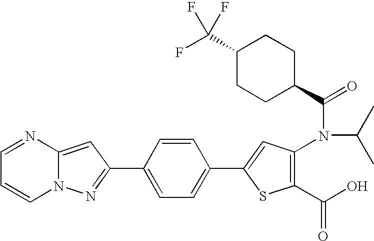

A solution of Intermediate 59 (120 mg) and 2M lithium hydroxide (1 mL) in methanol (3 mL) was stirred at room temperature for 4 days and was evaporated in vacuo. The residue was taken in to water, acidified with 2M HCl and was extracted with EtOAc (×2). The organics were separated, dried using a hydrophobic frit and evaporated in vacuo. The crude material was purified by NH₂ SPE cartridge, eluting with MeOH (×2) and 10% AcOH in MeOH (×2), followed by MDAP HPLC to give the title compound.

MS calcd for (C₂₈H₂₇F₃N₄O₃S+H)⁺: 557

MS found (electrospray): (M+H)⁺=557

¹H NMR (d₆-DMSO) δ 13.38 (1H, br), 9.17 (1H, d), 8.57 (1H, dd), 8.16 (2H, d), 7.96 (2H, d), 7.67 (1H, s), 7.36 (1H, s), 7.08 (1H, dd), 4.77 (1H, q), 2.29-2.15 (1H, m), 2.09 (1H, tt), 1.86-1.69 (4H, m), 1.64-1.51 (1H, m), 1.41-1.27 (1H, m), 1.16 (3H, d), 1.11-0.96 (2H, m), 0.92 (3H, d).

### Example 16

### 5-[4-(4-Amino-1,3-benzoxazol-2-yl)phenyl]-3-[[(trans-4-methylcyclohexyl)carbonyl](1-methylethyl)amino]-2-thiophenecarboxylic acid

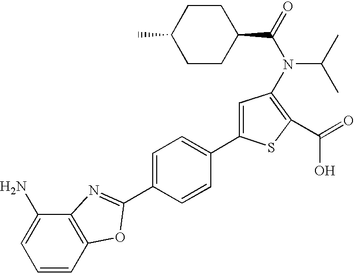

A solution of Intermediate 63 (637 mg) in THF (3 mL) and MeOH (3 mL) was stirred at 21° C. and was treated with 2N sodium hydroxide solution (3 mL). The resulting suspension was stirred overnight. The reaction was concentrated in vacuo and the residue was partitioned between water and EtOAc. The aqueous phase was separated and acidified to pH 2 with 2M HCl and was extracted into EtOAc (×3). The combined organics were washed with brine, dried over sodium sulphate and were evaporated in vacuo. The crude material was purified by ISCO Companion silica chromatography, eluting with a gradient of 0-20% MeOH in DCM to give the title compound.

MS calcd for (C₂₉H₃₁N₃O₄S+H)⁺: 518

MS found (electrospray): (M+H)⁺=518

¹H NMR (CD₃OD) δ 8.28 (2H, d), 7.95 (2H, d), 7.53 (1H, s), 7.14 (1H, t), 6.91 (1H, d), 6.65 (1H, d), 4.88-4.84 (1H, m), 2.15-2.06 (1H, m), 1.81-1.52 (5H, m), 1.46-1.27 (2H, m), 1.25 (3H, d), 1.01 (3H, d), 0.79 (3H, d), 0.76-0.56 (2H, m), carboxylic acid and amino protons not seen.

### Example 17

### 3-[[(trans-4-Methylcyclohexyl)carbonyl](1-methylethyl)amino]-5-(4-pyrazolo[1,5-a]pyrimidin-5-ylphenyl)-2-thiophenecarboxylic acid

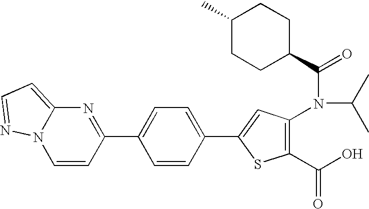

To a stirred solution of Intermediate 67 (130 mg) in THF (2 mL) and ethanol (2 mL) was added 2N sodium hydroxide solution (0.5 mL) and the mixture was stirred at room temperature for 22 h. The reaction mixture was evaporated in vacuo and then the residue was stirred between water and DCM. The whole mixture was evaporated in vacuo and the residue was partially dissolved in DMSO/MeOH. The resulting suspension was poured onto water and the mixture was filtered. The solid was dried in vacuo to give the title compound.

MS calcd for (C₂₈H₃₀N₄O₃S+H)⁺: 503

MS found (electrospray): (M+H)⁺=503

¹H NMR (d₆-DMSO) δ 13.39 (1H, br), 9.18 (1H, d), 8.29 (2H, d), 8.22 (1H, s), 7.97 (2H, d), 7.72-7.65 (2H, m), 6.76 (1H, s), 4.73 (1H, q), 1.94 (1H, t), 1.66-1.38 (5H, m), 1.27-1.14 (2H, m), 1.12 (3H, d), 0.87 (3H, d), 0.70 (3H, d), 0.66-0.44 (2H, m).

### Example 18

### 5-(6-Furo[3,2-b]pyridin-2-yl-3-pyridinyl)-3-[[(trans-4-methylcyclohexyl)carbonyl](1-methylethyl)amino]-2-thiophenecarboxylic acid

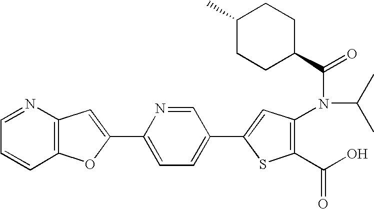

To a solution of Intermediate 122 (20 mg) in THF (1 mL) and MeOH (2 mL) was added 2N lithium hydroxide (1 mL) and the reaction was left to stir at room temperature for 48 h. The solvent was evaporated in vacuo and the residue was acidified to pH 1 with 2N HCl. The resulting suspension was applied to an OASIS HLB cartridge which was eluted with water (3 column volumes) and MeOH (3 volumes). Product containing fractions were evaporated in vacuo and were freeze dried from 1,4-dioxane to give the title compound.

MS calcd for (C₂₈H₂₉N₃O₄S+H)⁺: 504

MS found (electrospray): (M+H)⁺=504

¹H NMR (d₆-DMSO) δ 13.50 (1H, br), 9.22 (1H, m), 8.60 (1H, dd), 8.44 (1H, dd), 8.19-8.12 (2H, m), 7.82 (2H, d), 7.47-7.42 (1H, m), 4.77 (1H, q), 2.03-1.93 (1H, m), 1.69-1.42 (5H, m), 1.30-1.19 (2H, m), 1.16 (3H, d), 0.91 (3H, d), 0.75 (3H, d), 0.71-0.49 (2H, m).

### Example 19

### 5-[4-(7-Hydroxy-1-benzofuran-2-yl)phenyl]-3-[[(trans-4-methylcyclohexyl)carbonyl](1-methylethyl)amino]-2-thiophenecarboxylic acid

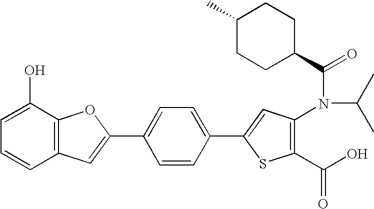

To a stirred solution of Intermediate 71 (78 mg) in THF (1 mL) and ethanol (4 mL) was added 2N sodium hydroxide (0.5 mL) and the resultant solution was stirred at room temperature for 0.5 h and was then allowed to stand for 20 h. The mixture was evaporated in vacuo and the residue was partitioned between DCM and 2N hydrochloric acid. The organics were separated using a hydrophobic frit and were evaporated in vacuo. The crude material was purified by MDAP HPLC to give the title compound.

MS calcd for (C₃₀H₃₁NO₅S+H)⁺: 518

MS found (electrospray): (M+H)⁺=518

¹H NMR (d₆-DMSO) δ 13.38 (1H, br), 10.14 (1H, s), 8.09-7.93 (4H, m), 7.65 (1H, s), 7.51 (1H, s), 7.15-7.02 (2H, m), 6.79 (1H, d), 4.76 (1H, q), 2.04-1.93 (1H, m), 1.69-1.41 (5H, m), 1.32-1.18 (2H, m), 1.16 (3H, d), 0.91 (3H, d), 0.74 (3H, d), 0.72-0.49 (2H, m).

### Example 20

### 5-[4-(7-Hydroxy-1,3-benzoxazol-2-yl)phenyl]-3-[[(trans-4-methylcyclohexyl)carbonyl](1-methylethyl)amino]-2-thiophenecarboxylic acid

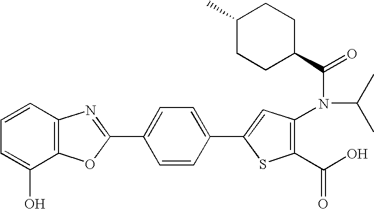

To solution of Intermediate 73 (52 mg) in ethanol (2 mL) and THF (2 mL) was added 2N sodium hydroxide solution (1 mL) and the solution was stirred at room temperature for 21 h. The reaction mixture was concentrated in vacuo to approximately 1 mL volume and was partitioned between 2N HCl and DCM. The organics were separated using a hydrophobic frit and were evaporated in vacuo. The crude material was purified by MDAP HPLC to give the title compound.

MS calcd for (C₂₉H₃₀N₂O₅S+H)⁺: 519

MS found (electrospray): (M+H)⁺=519

¹H NMR (CD₃OD) δ 8.32 (2H, d), 7.97 (2H, d), 7.52 (1H, s), 7.23-7.17 (2H, m), 6.88-6.83 (1H, m), 4.86 (1H, m), 2.17-2.07 (1H, m), 1.85-1.50 (5H, m), 1.45-1.31 (2H, m), 1.24 (3H, d), 1.01 (3H, d), 0.78 (3H, d), 0.75-0.55 (2H, m), carboxylic acid and phenolic protons not seen.

### Example 21

### 5-(4-Imidazo[2,1-b][1,3]thiazol-6-ylphenyl)-3-((1-methylethyl){[trans-4-(trifluoromethyl)cyclohexyl]carbonyl}amino)-2-thiophenecarboxylic acid

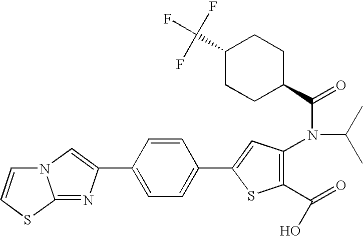

To solution of Intermediate 77 (84 mg) in methanol (0.5 mL) and THF (0.5 mL) was added 2N sodium hydroxide solution (0.4 mL) and the solution was stirred at room temperature overnight. The reaction mixture was concentrated in vacuo and was partitioned between 2N HCl and DCM. The organics were separated and were evaporated in vacuo. The crude material was purified by NH₂ SPE ion exchange cartridge to give the title compound.

MS calcd for (C₂₇H₂₆F₃N₃O₃S₂+H)⁺: 562

MS found (electrospray): (M+H)⁺=562

¹H NMR (CD₃OD) δ 8.56 (1H, s), 8.23 (1H, d), 8.04 (2H, d), 7.95 (2H, d), 7.75 (1H, d), 7.60 (1H, s), 4.94-4.90 (1H, m), 2.29-2.05 (2H, m), 2.02-1.85 (4H, m), 1.77-1.62 (1H, m), 1.57-1.42 (1H, m), 1.31 (3H, d), 1.23-1.09 (2H, m), 1.07 (3H, d), carboxylic acid proton not seen.

### Example 22

### 5-(4-Furo[3,2-b]pyridin-2-ylphenyl)-3-((1-methylethyl){[trans-4-(trifluoromethyl)cyclohexyl]carbonyl}amino)-2-thiophenecarboxylic acid

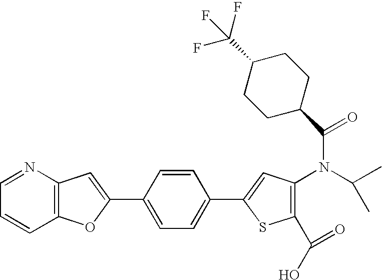

To solution of Intermediate 78 (120 mg) in methanol (0.5 mL) and THF (0.5 mL) was added 2N sodium hydroxide solution (0.5 mL) and the solution was stirred at room temperature overnight. The reaction mixture was concentrated in vacuo and was partitioned between 2N HCl and DCM. The organics were separated and were evaporated in vacuo. The crude material was purified by NH₂ SPE ion exchange cartridge to give the title compound.

MS calcd for (C₂₉H₂₇F₃N₂O₄S+H)⁺: 557

MS found (electrospray): (M+H)⁺=557

¹H NMR (CD₃OD) δ 8.73 (2H, d), 8.25 (2H, d), 8.03 (2H, d), 7.90-7.84 (1H, m), 7.81 (1H, s), 7.60 (1H, s), 4.88-4.84 (1H, m), 2.21-2.01 (2H, m), 1.95-1.80 (4H, m), 1.70-1.57 (1H, m), 1.51-1.37 (1H, m), 1.25 (3H, d), 1.19-0.97 (2H, m), 1.01 (3H, d), carboxylic acid proton not seen.

### Example 23

### 5-[4-(6-Amino-1,3-benzoxazol-2-yl)phenyl]-3-[[(trans-4-methylcyclohexyl)carbonyl](1-methylethyl)amino]-2-thiophenecarboxylic acid

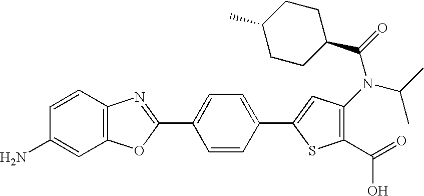

Intermediate 81 (384 mg) was dissolved in methanol (2 mL) and THF (2 mL) and was treated with 2N sodium hydroxide solution (2.2 mL). The mixture was stirred at room temperature for 4 h and was evaporated in vacuo. The residue was partitioned between EtOAc and water and the aqueous was extracted into EtOAc (×2). The aqueous layer was acidified to pH 3 with 2M HCl and was extracted with EtOAc (×3). The combined organics were dried over magnesium sulphate and were evaporated in vacuo. The crude material was purified by ISCO Companion silica chromatography, eluting with a gradient 0-100% methanol in DCM to give the title compound.

MS calcd for (C₂₉H₃₁N₃O₄S+H)⁺: 518

MS found (electrospray): (M+H)⁺=518

¹H NMR (CD₃OD) δ 8.20 (2H, d), 7.92 (2H, d), 7.50 (1H, s), 7.43 (1H, d), 6.94 (1H, d), 6.79 (1H, dd), 4.89-4.82 (1H, m), 2.16-2.05 (1H, m), 1.83-1.50 (5H, m), 1.45-1.26 (2H, m), 1.24 (3H, d), 1.00 (3H, d), 0.78 (3H, d), 0.75-0.55 (2H, m), carboxylic acid and amine protons not seen.

### Example 24

### 3-[[(trans-4-Methylcyclohexyl)carbonyl](1-methylethyl)amino]-5-(4-pyrazolo[1,5-b]pyridazin-2-ylphenyl)-2-thiophenecarboxylic acid

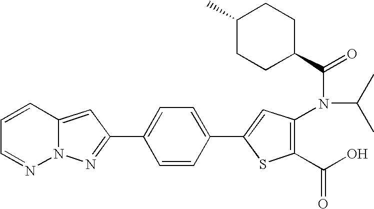

To a stirred solution of Intermediate 84 (56 mg) in THF (2 mL) and ethanol (2 mL) was added 2N sodium hydroxide (1 mL) and the resultant solution was stirred at room temperature for 0.5 h and was then allowed to stand for 20 h. The mixture was evaporated in vacuo and the residue was partitioned between DCM and 2N HCl. The organics were separated using a hydrophobic frit and were evaporated in vacuo. The residue was freeze-dried from 1,4-dioxane to give the title compound.

MS calcd for (C₂₈H₃₀N₄O₃S+H)⁺: 503

MS found (electrospray): (M+H)⁺=503

¹H NMR (CD₃OD) δ 8.36 (1H, dd), 8.17 (1H, dd), 8.11 (2H, d), 7.87 (2H, d), 7.44 (1H, s), 7.19-7.14 (2H, m), 4.88-4.83 (1H, m), 2.13 (1H, dt), 1.83-1.50 (5H, m), 1.46-1.27 (2H, m), 1.25 (3H, d), 1.01 (3H, d), 0.78 (3H, d), 0.76-0.58 (2H, m), carboxylic acid proton not seen.

### Example 25

### 3-((1-Methylethyl){[trans-4-(trifluoromethyl)cyclohexyl]carbonyl}amino)-5-(4-[1,3]oxazolo[4,5-b]pyridin-2-ylphenyl)-2-thiophenecarboxylic acid

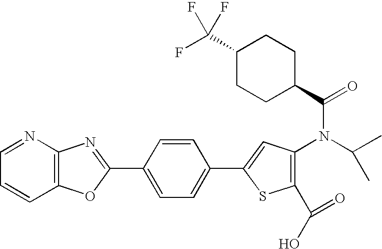

Intermediate 85 was dissolved in pyridine (1 mL) and was treated with lithium iodide (113 mg). The mixture was heated in a Reacti-vial at 110° C. overnight. Lithium iodide (45 mg) was added to the reaction and this was heated at 125° C. overnight. The mixture was evaporated in vacuo and the residue was partitioned between DCM and 2M HCl. The aqueous was separated in extracted with DCM. The combined organics were evaporated in vacuo and were purified by ISCO Companion silica chromatography, eluting with a gradient 0-100% MeOH in DCM. The product was then purified by an NH₂ SPE ion exchange cartridge, followed by ISCO Companion silica chromatography, eluting with a gradient 0-100% MeOH in DCM, followed by MDAP HPLC to give the title compound.

MS calcd for (C₂₈H₂₆F₃N₃O₄S+H)⁺: 558

MS found (electrospray): (M+H)⁺=558

¹H NMR (CD₃OD) δ 8.55 (1H, d), 8.42 (2H, d), 8.19 (1H, dd), 8.07-8.02 (2H, m), 7.61 (1H, s), 7.50 (1H, dd), 4.87-4.83 (1H, m), 2.23-1.97 (2H, m), 1.95-1.80 (3H, m), 1.70-1.56 (1H, m), 1.50-1.36 (1H, m), 1.25 (3H, d), 1.17-1.04 (2H, m), 1.01 (3H, d), 1.00-0.95 (1H, m), carboxylic acid proton not seen.

### Example 26

### 3-((1-Methylethyl){[trans-4-(trifluoromethyl)cyclohexyl]carbonyl}amino)-5-[4-(5-methylpyrazolo[1,5-a]pyrimidin-2-yl)phenyl]-2-thiophenecarboxylic acid

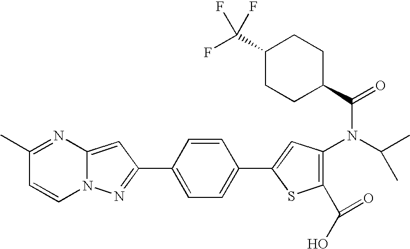

Intermediate 86 (106 mg) in THF (0.5 mL) and MeOH (0.5 mL) was treated with 2N sodium hydroxide solution (0.5 mL) and the mixture was stirred at room temperature overnight. The reaction was evaporated in vacuo and was partitioned between DCM and 2M HCl. The organics were separated and washed with 2M HCl and the aqueous layer was extracted with DCM. The combined organics were evaporated in vacuo and were purified using an NH₂ SPE ion exchange cartridge to give the title compound.

MS calcd for (C₂₉H₂₉F₃N₄O₃S+H)⁺: 571

MS found (electrospray): (M+H)⁺=571

¹H NMR (CD₃OD) δ 8.95 (1H, d), 8.13 (2H, d), 7.89 (2H, d), 7.49 (1H, s), 7.05 (1H, s), 7.01 (1H, d), 4.87-4.83 (1H, m), 2.67 (3H, s), 2.19 (1H, tt), 2.12-1.99 (1H, m), 1.95-1.80 (4H, m), 1.70-1.55 (1H, m), 1.50-1.36 (1H, m), 1.25 (3H, d), 1.18-1.03 (2H, m), 1.01 (3H, d), carboxylic acid proton not seen.

### Example 27

### 5-[4-(6-Aminoimidazo[1,2-a]pyridin-2-yl)phenyl]-3-[[(trans-4-methylcyclohexyl)carbonyl](1-methylethyl)amino]-2-thiophenecarboxylic acid

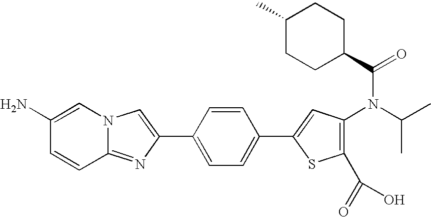

Intermediate 90 (126 mg) in THF (1 mL) and MeOH (1 mL) was treated with 2N sodium hydroxide solution (0.71 mL) and the mixture was stirred at room temperature for 18 h. The reaction was evaporated in vacuo and was partitioned between EtOAc and water. This gave an emulsion. The aqueous phase was acidified to pH 3 and the organics were extracted into EtOAc (×3). The organics were combined, dried over magnesium sulphate and were evaporated in vacuo. Some solid remained at the phase interface and this was filtered off. The solid was dissolved in MeOH and was combined with the rest of the organic material. This was purified by silica SPE cartridge eluting with stepped solvents; 2% MeOH in DCM to 20% MeOH in DCM, followed by MeOH. The product was further purified using MDAP HPLC to give the title compound.

MS calcd for (C₂₉H₃₂N₄O₃S+H)⁺: 517

MS found (electrospray): (M+H)⁺=517

¹H NMR (CD₃OD) δ 8.40 (1H, s), 7.99-7.91 (5H, m), 7.65 (1H, d), 7.51 (2H, dd), 4.89-4.84 (1H, m), 2.10 (1H, tt), 1.82-1.51 (5H, m), 1.45-1.26 (2H, m), 1.24 (3H, d), 1.01 (3H, d), 0.78 (3H, d), 0.76-0.55 (2H, m), carboxylic acid and amine protons not seen.

### Example 28

### 5-[4-(1H-Benzimidazol-5-yl)phenyl]-3-[[(trans-4-methylcyclohexyl)carbonyl](1-methylethyl)amino]-2-thiophenecarboxylic acid

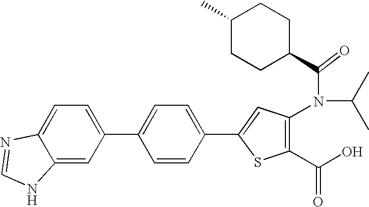

To a solution of Intermediate 92 (94 mg) in DCM (3 mL) was added TFA (1 mL) and the mixture was stirred at room temperature for 4 h. The reaction mixture was evaporated in vacuo and the residue was dissolved in THF (2 mL) and ethanol (2 mL). 2N sodium hydroxide solution (1 mL) was added and the mixture was stirred for 0.5 h and was left to stand for 20 h. The reaction was neutralised with 2M HCl and the mixture was evaporated in vacuo. The crude material was purified by MDAP HPLC and was freeze-dried from 1,4-dioxane to give the title compound.

MS calcd for (C₂₉H₃₁N₃O₃S+H)⁺: 502

MS found (electrospray): (M+H)⁺=502

¹H NMR (d₆-DMSO) δ 12.88 (1H, br), 8.28 (1H, s), 7.85-7.70 (3H, m), 7.82 (2H, d), 7.69 (1H, d), 7.61 (1H, s), 7.57 (1H, dd), 4.76 (1H, q), 1.99 (1H, tt), 1.69-1.41 (5H, m), 1.31-1.18 (2H, m), 1.15 (3H, d), 0.91 (3H, d), 0.74 (3H, d), 0.72-0.49 (2H, m), carboxylic acid proton not seen.

### Example 29

### 5-[4-(5-Amino-1-benzofuran-2-yl)phenyl]-3-[[(trans-4-methylcyclohexyl)carbonyl](1-methylethyl)amino]-2-thiophenecarboxylic acid

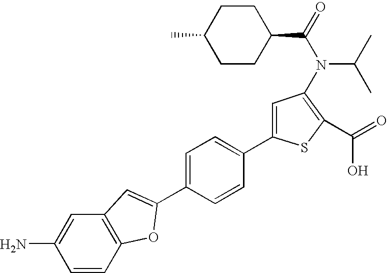

A solution of Intermediate 97 (158 mg) in MeOH (1.5 mL) and THF (1.5 mL) was treated with 2M NaOH solution and was stirred at room temperature for 24 h. The mixture was evaporated in vacuo. The residue was partitioned between water (100 mL) and EtOAc (100 mL) and the aqueous was acidified with 2M HCl solution. The organics were separated, dried over magnesium sulphate and evaporated in vacuo. The crude material was purified by ISCO Companion silica chromatography, eluting with a gradient 0-55% MeOH in EtOAc to give the title compound.

MS calcd for (C₃₀H₃₂N₂O₄S+H)⁺: 517

MS found (electrospray): (M+H)⁺=517

¹H NMR (CD₃OD) δ 7.90 (2H, d), 7.79 (2H, d), 7.31 (1H, s), 7.29 (1H, d), 7.06 (1H, s), 6.93 (1H, d), 6.77 (1H, dd), 4.88-4.82 (1H, m), 2.26-2.15 (1H, m), 1.93-1.82 (1H, m), 1.72-1.47 (4H, m), 1.40-1.25 (2H, m), 1.23 (3H, d), 1.03 (3H, d), 0.77 (3H, d), 0.55-0.75 (2H, m), carboxylic acid and amine protons not seen.

### Example 30

### 5-[4-(6-Amino-1-benzofuran-2-yl)phenyl]-3-[[(trans-4-methylcyclohexyl)carbonyl](1-methylethyl)amino]-2-thiophenecarboxylic acid

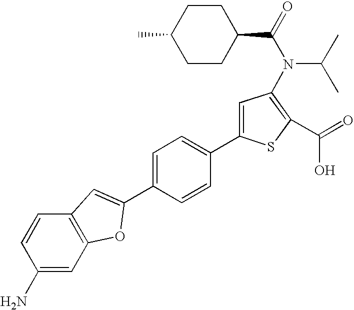

A solution of Intermediate 104 (152 mg) in MeOH (1.5 mL) and THF (1.5 mL) was treated with 2M NaOH solution and was stirred at room temperature for 24 h. The mixture was evaporated in vacuo. The residue was dissolved in water (100 mL), was acidified with 2M HCl solution and was extracted into EtOAc (100 mL). The organics were separated, dried over magnesium sulphate and evaporated in vacuo. The crude material was purified by ISCO Companion silica chromatography, eluting with a gradient 0-50% MeOH in EtOAc to give the title compound.

MS calcd for (C₃₀H₃₂N₂O₄S+H)⁺: 517

MS found (electrospray): (M+H)⁺=517

¹H NMR (CD₃OD) δ 7.86 (2H, d), 7.77 (2H, d), 7.32 (1H, s), 7.31 (1H, d), 7.09 (1H, s), 6.86 (1H, br), 6.69 (1H, dd), 4.88-4.83 (1H, m), 2.23-2.12 (1H, m), 1.89-1.79 (1H, m), 1.75-1.48 (4H, m), 1.25-1.40 (2H, m), 1.23 (3H, d), 1.02 (3H, d), 0.78 (3H, d), 0.55-0.75 (2H, m), carboxylic acid and amine protons not seen.

### Example 31

### 5-[5-(1,3-Benzoxazol-2-yl)-3-furanyl]-3-[[(trans-4-methylcyclohexyl)carbonyl](1-methylethyl)amino]-2-thiophenecarboxylic acid

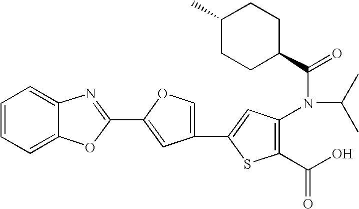

A solution of Intermediate 139 (74 mg) in ethanol (1.5 mL) and THF (1.5 mL) was treated with 2N sodium hydroxide solution (1 mL). The reaction was left to stir at room temperature overnight. DCM (12 mL) was added and the mixture was acidified with 2N HCl. The reaction was left to stir for 30 mins and was then separated using a hydrophobic frit. The organics were evaporated in vacuo and the crude material was purified by MDAP HPLC to give the title compound.

MS calcd for (C₂₇H₂₈N₂O₅S+H)⁺: 493

MS found (electrospray): (M+H)⁺=493

¹H NMR (CDCl₃) δ 8.06 (1H, s), 7.89-7.83 (1H, m), 7.64-7.59 (1H, m), 7.57 (1H, s), 7.46-7.40 (2H, m), 7.03 (1H, s), 5.01 (1H, br), 2.14-2.03 (1H, m), 1.80-1.56 (5H, m), 1.55-1.41 (1H, m), 1.40-1.28 (1H, m), 1.24 (3H, d), 1.02 (3H, d), 0.79 (3H, d), 0.76-0.60 (2H, m), carboxylic acid proton not seen.

### Example 32

### 5-(6-Imidazo[2,1-b][1,3]thiazol-6-yl-3-pyridinyl)-3-[[(trans-4-methylcyclohexyl)carbonyl](1-methylethyl)amino]-2-thiophenecarboxylic acid

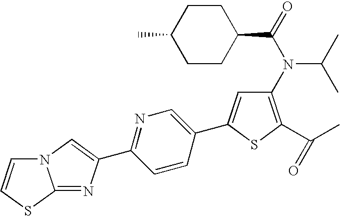

A solution of Intermediate 109 (32 mg) and 2M lithium hydroxide solution (1 mL) in methanol (2 mL) was stirred at room temperature for 3 days and was evaporated in vacuo. The residue was acidified to pH6 with 2M HCl and was diluted with water. This solution was extracted with DCM (×2), the combined organics were dried using a hydrophobic frit and the solvent was evaporated in vacuo. The crude material was purified using an NH₂ SPE ion exchange cartridge, eluting with MeOH (3 volumes) and 10% AcOH/MeOH (3 column volumes). The material was purified further by MDAP HPLC, and was freeze dried from 1,4-dioxane to give the title compound.

MS calcd for (C₂₆H₂₈N₄O₃S₂+H)⁺: 509

MS found (electrospray): (M+H)⁺=509

¹H NMR (CD₃OD) δ 8.92 (1H, d), 8.34 (1H, s), 8.24-8.20 (1H, m), 8.05 (1H, d), 7.86 (1H, d), 7.50 (1H, s), 7.23 (1H, d), 4.90-4.86 (1H, m), 2.17 (1H, tt), 1.89-1.51 (5H, m), 1.46-1.28 (2H, m), 1.26 (3H, d), 1.04 (3H, d), 0.80 (3H, d), 0.78-0.59 (2H, m) carboxylic acid proton not seen.

### Example 33

### 5-[4-(7-Aminopyrazolo[1,5-a]pyrimidin-2-yl)-3-chlorophenyl]-3-[[(trans-4-methylcyclohexyl)carbonyl](1-methylethyl)amino]-2-thiophenecarboxylic acid

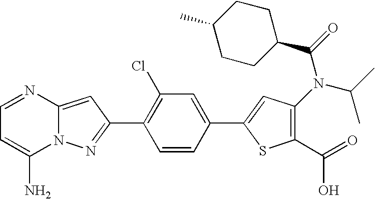

Intermediate 115 (290 mg) was dissolved in THF (5 mL) and MeOH (5 mL). 2N sodium hydroxide solution (2.5 mL) was added and the mixture was stirred at room temperature for 48 h. The reaction was acidified with 2N HCl and was extracted with EtOAc. The organics were dried over sodium sulphate and concentrated. The crude material was purified by MDAP HPLC to give the title compound.

MS calcd for (C₂₈H₃₀ClN₅O₃S+H)⁺: 552/554

MS found (electrospray): (M+H)⁺=552/554

¹H NMR (d₆-DMSO) δ 8.15-8.12 (2H, m), 8.11 (1H, d), 7.89 (1H, dd), 7.77 (1H, s), 6.90 (1H, s), 6.16 (1H, d), 4.75 (1H, q), 1.97 (1H, tt), 1.70-1.41 (5H, m), 1.31-1.18 (2H, m), 1.16 (3H, d), 0.91 (3H, d), 0.75 (3H, d), 0.72-0.48 (2H, m), carboxylic acid and amine protons not seen.

### Example 34

### 3-[[(trans-4-Methylcyclohexyl)carbonyl](1-methylethyl)amino]-5-(6-pyrazolo[1,5-a]pyrimidin-2-yl-3-pyridinyl)-2-thiophenecarboxylic acid

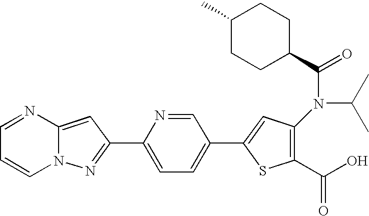

To a solution of Intermediate 119 (43 mg) in THF (1 mL) and MeOH (2 mL) was added 2N lithium hydroxide solution (1 mL) and the mixture was stirred at room temperature for 2 days. The solvent was evaporated in vacuo and the residue was acidified to pH 1 with 2N HCl. The resulting suspension was applied to an OASIS HLB cartridge which was eluted with water (3 column volumes) and MeOH (3 column volumes). Product containing fractions were evaporated in vacuo and were freeze dried from 1,4-dioxane to give the title compound.

MS calcd for (C₂₇H₂₉N₅O₃S+H)⁺: 504

MS found (electrospray): (M+H)⁺=504

¹H NMR (d₆-DMSO) δ 13.59 (1H, br), 9.20 (1H, d), 9.14 (1H, d), 8.60 (1H, dd), 8.36 (1H, dd), 8.22 (1H, d), 7.71 (1H, s), 7.28 (1H, s), 7.13 (1H, dd), 4.75 (1H, q), 2.06-1.95 (1H, m), 1.70-1.39 (5H, m), 1.30-1.18 (2H, m), 1.15 (3H, d), 0.91 (3H, d), 0.74 (3H, d), 0.72-0.47 (2H, m).

### Example 35

### 3-{(Cyclopropylmethyl)[(trans-4-methylcyclohexyl)carbonyl]amino}-5-(4-pyrazolo[1,5-a]pyrimidin-2-ylphenyl)-2-thiophenecarboxylic acid

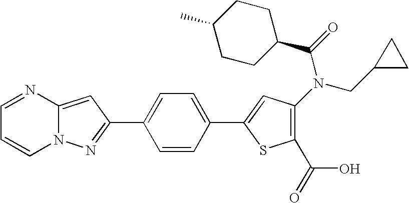

To a solution of Intermediate 130 (560 mg) in MeOH (1.5 mL) and THF (1.5 mL) was added 2N sodium hydroxide solution (1.5 mL) and the reaction was stirred at room temperature for 18 h. The reaction was acidified with 1N HCl (3 mL) and was evaporated in vacuo. The residue was partitioned between DCM and water and the organics were separated by hydrophobic frit. The organics were evaporated in vacuo. The crude material was purified by reverse phase ISCO Companion chromatography, using a C18 cartridge, eluting with a gradient 5% MeCN (0.05% formic acid)/water (0.1% formic acid) to 100% MeCN (0.05% formic acid), and was freeze-dried from 1,4-dioxane to give the title compound.

MS calcd for (C₂₉H₃₀N₄O₃S+H)⁺: 515

MS found (electrospray): (M+H)⁺=515

¹H NMR (CDCl₃) δ 8.76 (1H, dd), 8.52 (1H, dd), 8.10 (2H, d), 7.79 (2H, d), 7.30 (1H, s), 7.07 (1H, s), 6.88 (1H, dd), 3.93-3.83 (1H, m), 3.43-3.33 (2H, m), 2.28-2.15 (1H, m), 1.80-1.50 (6H, m), 1.41-1.27 (1H, m), 1.05-0.93 (1H, m), 0.84-0.65 (4H, m), 0.45 (2H, d), 0.11 (2H, br t), carboxylic acid proton not seen.

### Example 36

### 4-[[(trans-4-Methylcyclohexyl)carbonyl](1-methylethyl)amino]-5′-pyrazolo[1,5-a]pyrimidin-2-yl-2,2′-bithionhene-5-carboxylic acid

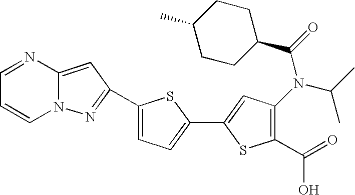

To a solution of methyl 4-[[(trans-4-methylcyclohexyl)carbonyl](1-methylethyl)amino]-5′-pyrazolo[1,5-a]pyrimidin-2-yl-2,2′-bithiophene-5-carboxylate (129 mg, a synthesis of which is described above as Intermediate 147) in THF (2 mL) and MeOH (2 mL) was added 2N lithium hydroxide (1 mL) and the reaction was left to stir at room temperature for 24 h. The solvent was evaporated in vacuo, the residue was acidified to pH 1.0 with 2N HCl and was extracted with EtOAc (10 mL×2). The combined organics were dried over sodium sulphate and evaporated in vacuo. The residue was partially dissolved in hot MeCN and was refluxed for 1 h. The mixture was allowed to cool and the solid was filtered off. The filtrate was evaporated, dissolved in hot MeOH (6 mL) and left to stand for 4 days. The solid obtained was filtered off and dried in air, then in a vacuum oven at 60° C. for 6 h to give the title compound.

MS calcd for (C₂₆H₂₈N₄O₃S₂+H)⁺: 509

MS found (electrospray): (M+H)⁺=509

¹H NMR (d₆-DMSO) δ 13.39 (1H, br), 9.11 (1H, d), 8.56 (1H, dd), 7.80 (1H, d), 7.69 (1H, d), 7.43 (1H, s), 7.24 (1H, d), 7.07 (1H, dd), 4.75 (1H, quintet), 2.03-1.93 (1H, m), 1.67-1.41 (5H, m), 1.31-1.18 (2H, m), 1.13 (3H, d), 0.91 (3H, d), 0.75 (3H, d), 0.72-0.51 (2H, m).

### Example 37

### Sodium 3-[[(trans-4-methylcyclohexyl)carbonyl](1-methylethyl)amino]-5-(4-pyrazolo[1,5-a]pyrimidin-2-ylphenyl)-2-thiophenecarboxylate

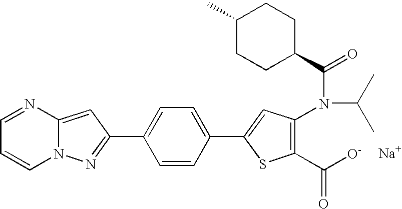

3-[[(trans-4-Methylcyclohexyl)carbonyl](1-methylethyl)amino]-5-(4-pyrazolo[1,5-a]pyrimidin-2-ylphenyl)-2-thiophenecarboxylic acid (598 mg, a synthesis of which is described above as Example 2) was slurried in MIBK (42 mL). 1M sodium hydroxide solution (1.2 mL) was added and the mixture was stirred for 2 mins. Gentle heating was applied with a heat gun, then the mixture was allowed to cool to room temperature and was stirred for 3 days. The solid was filtered off and dried in a vacuum oven at 70° C. for 24 h to give the title compound.

¹H NMR (d₆-DMSO) δ 9.15 (1H, d), 8.55 (1H, s), 8.09 (2H, d), 7.80 (2H, d), 7.30-7.25 (2H, m), 7.08-7.03 (1H, m), 4.74-4.66 (1H, m), 2.15 (1H, t), 1.83 (1H, d), 1.64-1.48 (3H, m), 1.38 (1H, q), 1.30-1.14 (2H, m), 1.11 (3H, d), 0.94 (3H, d), 0.74 (3H, d), 0.69-0.45 (2H, m).

### Alternative Preparation of Example 37 (Method B)

### Sodium 3-[[(trans-4-methylcyclohexyl)carbonyl](1-methylethyl)amino]-5-(4-pyrazolo[1,5-a]pyrimidin-2-ylphenyl)-2-thiophenecarboxylate

A solution of 3-[[(trans-4-methylcyclohexyl)carbonyl](1-methylethyl)amino]-5-(4-pyrazolo[1,5-a]pyrimidin-2-ylphenyl)-2-thiophenecarboxylic acid (10.33 g, a synthesis of which is described above as Example 2) in iso-propanol (300 mL) was treated with 2N sodium hydroxide (10 mL) dropwise at ambient temperature. The mixture was stirred overnight and the suspension was isolated by filtration. The solid was washed with iso-propanol (20 mL) and dried in vacuo to give the title compound.

¹H NMR (d₆-DMSO): δ 9.15 (1H, d), 8.54 (1H, m), 8.08 (2H, d), 7.81 (2H, d), 7.29 (2H, s), 7.05 (1H, m), 4.70 (1H, m), 2.15 (1H, bt), 1.65-1.49 (3H, m), 1.39 (1H, bq), 1.26-1.05 (5H, m & d (at 1.11)), 0.93 (3H, d), 0.72 (3H, d), 0.70-0.40 (2H, m).

### Example 38

### Ammonium 3-[[(trans-4-methylcyclohexyl)carbonyl](1-methylethyl)amino]-5-(4-pyrazolo[1,5-a]pyrimidin-2-ylphenyl)-2-thiophenecarboxylate

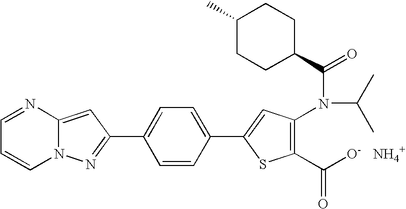

3-[[(trans-4-Methylcyclohexyl)carbonyl](1-methylethyl)amino]-5-(4-pyrazolo[1,5-a]pyrimidin-2-ylphenyl)-2-thiophenecarboxylic acid (510 mg, a synthesis of which is described above as Example 2) was slurried in aqueous isopropyl alcohol (15 mL, 5% water) and this was stirred at room temperature for 5 mins. Ammonium hydroxide solution (0.62 mL, 28% in water) was added and the mixture was stirred for 3 days. The solid was filtered off and dried in a vacuum oven at 40° C. for 24 h to give the title compound.

¹H NMR (d₆-DMSO) δ 9.16 (1H, d), 8.56 (1H, dd), 8.10 (2H, d), 7.82 (2H, d), 7.58-7.10 (4H, br s+2H, d), 7.06 (1H, dd), 4.71 (1H, quintet), 2.13 (1H, tt), 1.79 (1H, d), 1.64-1.48 (3H, m), 1.46-1.33 (1H, m), 1.29-1.14 (2H, m), 1.11 (3H, d), 0.93 (3H, d), 0.74 (3H, d), 0.70 (2H, m).

### Example 39

### 3-[{[(1S,2R,4S)-2-Hydroxy-4-methylcyclohexyl]carbonyl}(1-methylethyl)amino]-5-(4-pyrazolo[1,5-a]pyrimidin-2-ylphenyl)-2-thiophenecarboxylic acid

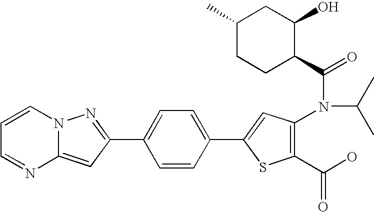

To a stirred solution of Intermediate 144 (72 mg) in THF (2 mL) and ethanol (2 mL) was added 2N sodium hydroxide solution (1 mL). The mixture was stirred for 0.5 h, left to stand for 3 days then evaporated in vacuo. The residue was partitioned between 2N HCl and DCM. The aqueous was separated by hydrophobic frit, extracted with DCM (×2) and the combined organics were evaporated in vacuo. The crude material was purified by MDAP HPLC and the material freeze-dried from 1,4-dioxane to give the title compound.

MS calcd for (C₂₈H₃₀N₄O₄S+H)⁺: 519

MS found (electrospray): (M+H)⁺=519

¹H NMR (d₆-DMSO, 392 K) δ 9.02 (1H, d), 8.53 (1H, d), 8.11 (2H, d), 7.86 (2H, d), 7.44 (1H, s), 7.17 (1H, s), 7.01 (1H, dd), 4.72 (1H, quintet), 4.01 (1H, br s), 2.30-2.18 (1H, br m). 1.93-0.53 (16H, m), carboxylic acid and hydroxyl protons not seen.

### Example 40

### Ammonium 3-{ethyl[(trans-4-methylcyclohexyl)carbonyl]amino}-5-(4-pyrazolo[1,5-a]pyrimidin-2-ylphenyl)-2-thiophenecarboxylate

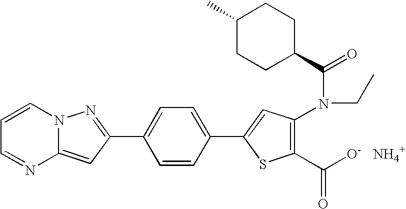

To a stirred solution of Intermediate 146 (0.58 g) in THF (5 mL) and ethanol (5 mL) was added 2N sodium hydroxide (2.5 mL). The mixture was allowed to stand at room temperature for 21 h, and was concentrated in vacuo to approximately 2 mL. The residue was partitioned between DCM and 2N HCl, then the organics were separated and evaporated in vacuo. To the crude material was added DMSO (5 mL) and MeOH (5 mL).

The MeOH was then evaporated in vacuo. Water (50 mL) was added and the solid was collected by filtration and was washed with water. The solid was dried and the crude material was pre-adsorbed onto a silica Biotage cartridge (loaded in 60:10:1 chloroform/MeOH/aqueous ammonia). The cartridge was eluted with 60:10:1 chloroform/MeOH/aqueous ammonia to give the title compound.

MS calcd for (C₂₇H₂₈N₄O₃S+H)⁺: 489

MS found (electrospray): (M+H)⁺=489

¹H NMR (d₆-DMSO, 392 K) δ 9.02 (1H, ddd), 8.53 (1H, dd), 8.10 (2H, dt), 7.83 (2H, dt), 7.44 (1H, s), 7.16 (1H, d), 7.01 (1H, dd), 3.68 (2H, br), 2.30-2.16 (1H, m), 1.78-1.17 (7H, m), 1.08 (3H, t), 0.80 (3H, d), 0.77-0.62 (2H, m), carboxylic acid proton not seen.

### Example 41

### 5-[4-(1H-Indol-5-yl)phenyl]-3-[[(trans-4-methylcyclohexyl)carbonyl](1-methylethyl)amino]-2-thiophenecarboxylic acid

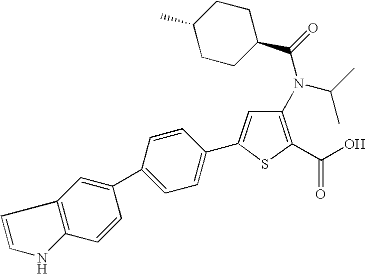

A mixture of Intermediate 154 (32.2 mg), 2M sodium hydroxide solution (0.31 mL), MeOH (0.31 mL) and THF (0.31 mL) was stirred for 20 h. The mixture was partitioned between 2M HCl (1 mL) and DCM (1 mL) and the organics were separated using a hydrophobic frit. The aqueous layer was washed with DCM (1 mL) and the combined organics were evaporated in vacuo. The material was freeze-dried from 1,4-dioxane to give the title compound.

MS calcd for (C₃₀H₃₂N₂O₃S+H)⁺: 502

MS found (electrospray): (M+H)⁺=502

¹H NMR (CD₃OD) δ 7.86 (1H, d), 7.82-7.72 (4H, m), 7.48-7.41 (2H, m), 7.37 (1H, s), 7.27 (1H, d), 6.51 (1H, d), 2.13 (1H, tt), 1.81-1.51 (5H, m), 1.45-1.20 (6H, m), 1.01 (3H, d), 0.78 (3H, d), 0.75-0.57 (2H, m), one proton not seen as obscured by water peak δ 4.88 and carboxylic acid proton not seen.

### Example 42

### 5-[4-(1H-Indol-6-yl)phenyl]-3-[[(trans-4-methylcyclohexyl)carbonyl](1-methylethyl)amino]-2-thiophenecarboxylic acid

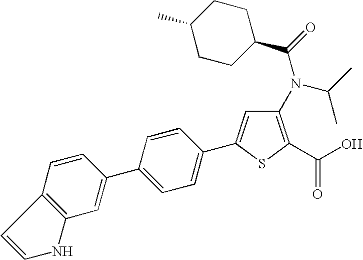

A mixture of Intermediate 155 (20 mg), 2M sodium hydroxide solution (0.19 mL), MeOH (0.19 mL) and THF (0.19 mL) was stirred for 12 h. The mixture was partitioned between 2M HCl (1 mL) and DCM (1 mL). The aqueous layer was extracted with DCM (1 mL) and the combined organics were evaporated in vacuo to give the title compound.

¹H NMR (CD₃OD) δ 7.83-7.74 (4H, m), 7.69 (1H, s), 7.62 (1H, d), 7.39-7.33 (2H, m), 7.28 (1H, d), 6.46 (1H, d), 2.13 (1H, tt), 1.81-1.51 (6H, m), 1.45-1.26 (2H, m), 1.24 (3H, d), 1.01 (3H, d), 0.79 (3H, d), 0.76-0.58 (2H, m), indole proton not seen and one proton not seen as obscured by water peak δ 4.87.

MS calcd for (C₃₀H₃₂N₂O₃S+H)⁺: 502

MS found (electrospray): (M+H)⁺=502

### Example 43

### 5′-Furo[3,2-b]pyridin-2-yl-4-[[(trans-4-methylcyclohexyl)carbonyl](1-methylethyl)amino]-2,2′-bithiophene-5-carboxylic acid

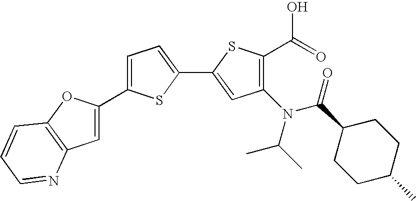

Intermediate 151 (170 mg) was dissolved in MeOH (3 mL) and THF (3 mL). 2N sodium hydroxide (1.5 mL) was added and the reaction was stirred at room temperature for 3 days.

The reaction was diluted with DCM and 2N HCL was added. The mixture was stirred at room temperature for 30 mins. The organics were separated using a hydrophobic frit and were evaporated in vacuo. The crude material was purified by reverse phase ISCO

Companion chromatography, using a C18 cartridge, eluting with a gradient 40% MeCN (0.05% formic acid)/water (0.1% formic acid) to 95% MeCN (0.05% formic acid) to give the title compound.

MS calcd for (C₂₇H₂₈N₂O₄S₂+H)⁺: 509

MS found (electrospray): (M+H)⁺=509

¹H NMR (CDCl₃) δ 8.57 (1H, dd), 7.83 (1H, d), 7.61 (1H, d), 7.41 (1H, d), 7.30 (1H, dd), 7.25 (1H, s), 7.04 (1H, s), 5.02 (1H, br), 2.21-2.09 (1H, m), 1.85-1.59 (5H, m), 1.56-1.43 (1H, m), 1.41-1.29 (1H, m), 1.25 (3H, d), 1.05 (3H, d), 0.80 (3H, d), 0.78-0.63 (2H, m), carboxylic acid proton not seen.

### Example 44

### Potassium 5-(4-furo[3,2-b]pyridin-2-ylphenyl)-3-[[(trans-4-methylcyclohexyl)carbonyl](1-methylethyl)amino]-2-thiophenecarboxylate

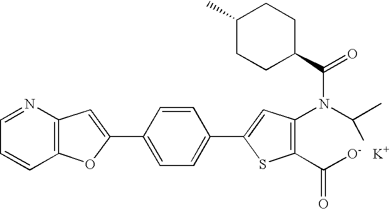

MIBK (20 mL) was added to 5-(4-furo[3,2-b]pyridin-2-ylphenyl)-3-[[(trans-4-methylcyclohexyl)carbonyl](1-methylethyl)amino]-2-thiophenecarboxylic acid (200 mg, a synthesis of which is described above as Example 1). 1M potassium hydroxide (0.398 mL) was added and the reaction was stirred at room temperature for 3 days. The solid was collected by vacuum filtration and was dried at 70° C. in a vacuum oven for 48 h to give the title compound.

¹H NMR (d₆-DMSO) δ 8.25 (1H, dd), 8.06 (1H, d), 8.02 (2H, d), 7.83 (2H, d), 7.70 (1H, s), 7.34 (1H, dd), 7.28 (1H, s), 4.69 (1H, quintet), 2.15 (1H, tt), 1.83 (1H, d), 1.64-1.48 (3H, m), 1.44-1.31 (1H, m), 1.28-1.14 (2H, m), 1.11 (3H, d), 0.93 (3H, d), 0.74 (3H, d), 0.69-0.46 (2H, m).

### Example 45

### Trisamine 5-(4-furo[3,2-b]pyridin-2-ylphenyl)-3-[[(trans-4-methylcyclohexyl)carbonyl](1-methylethyl)amino]-2-thiophenecarboxylate

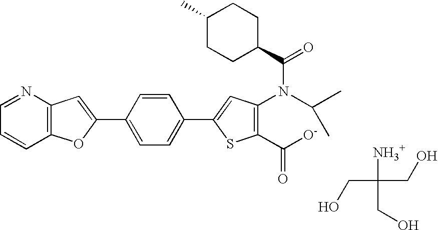

To 5-(4-furo[3,2-b]pyridin-2-ylphenyl)-3-[[(trans-4-methylcyclohexyl)carbonyl](1-methylethyl)amino]-2-thiophenecarboxylic acid (201.6 mg, a synthesis of which is described above as Example 1) and tris(hydroxymethyl)aminomethane (53.6 mg) was added MIBK (total 8 mL) in 1 mL and 2 mL portions, with heating applied using a heat gun between each addition. The mixture was then temperature cycled (40° C. for 4 h/room temperature for 4 h) for a total of 3 days. The solid was collected by filtration and dried at 70° C. in a vacuum oven to give the title compound.

¹H NMR (d₆-DMSO) δ 8.53 (1H, dd), 8.09-8.02 (3H, m), 7.88 (2H, d), 7.72 (1H, s), 7.39 (1H, s), 7.35 (1H, dd), 4.72 (1H, quintet), 3.48 (6H, s), 2.12 (1H, tt), 1.79 (1H, d), 1.65-1.49 (3H, m), 1.47-1.34 (1H, m), 1.36-1.16 (2H, m), 1.13 (3H, d), 0.94 (3H, d), 0.74 (3H, d), 0.71-0.46 (2H, m), exchangeable protons not clearly resolved.

### Example 46

### 3-[[(trans-4-Methylcyclohexyl)carbonyl](1-methylethyl)amino]-5-(6-[1,3]oxazolo[4,5-b]pyridin-2-yl-3-pyridinyl)-2-thiophenecarboxylic acid

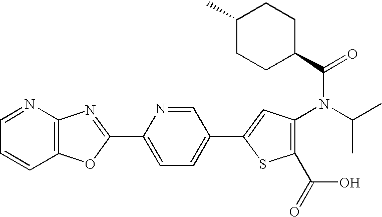

A mixture of Intermediate 159 (56 mg) and lithium iodide (72 mg) in pyridine (0.5 mL) was heated in a Reacti-vial at 120° C. for 20 h, and was evaporated in vacuo. The residue was taken into water and was extracted with EtOAc (×3). The organics were separated, dried using a hydrophobic frit and were evaporated in vacuo. The crude material was purified by MDAP HPLC, and was freeze dried from 1,4-dioxane to give the title compound.

MS calcd for (C₂₇H₂₈N₄O₄S+H)⁺: 505

MS found (electrospray): (M+H)⁺=505

¹H NMR (CD₃OD) δ 14.00-13.20 (1H, br), 9.29 (1H, d), 8.64 (1H, dd), 8.54-8.45 (2H, m), 8.35 (1H, dd), 7.87 (1H, s), 7.55 (1H, dd), 4.76 (1H, q), 2.04-1.94 (1H, m), 1.69-1.41 (5H, m), 1.30-1.18 (2H, m), 1.15 (3H, d), 0.92 (3H, d), 0.74 (3H, d), 0.72-0.49 (2H, m).

### Compound A

### 5-Phenyl-3-[[(trans-4-methylcyclohexyl)carbonyl](1-methylethyl)amino]-2-thiophenecarboxylic acid

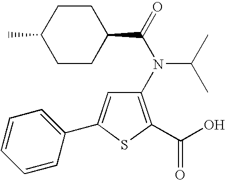

To a mixture of Intermediate 19 (390 mg) in THF/MeOH/water (3:2:1, vol/vol, 40 mL total) was added lithium hydroxide monohydrate (246 mg). The mixture was stirred at room temperature for 20 hours, the solvents removed in vacuo, and the residue partitioned between water (40 mL) and ethyl acetate (40 mL). The organic layer was dried (Na₂SO₄), evaporated and triturated with ether to give the title compound.

MS calcd for (C₂₂H₂₇NO₃S+H)⁺: 356

MS found (electrospray): (M+H)⁺=356

### Compound B

### 5-(4-Chlorophenyl)-3-[[(trans-4-methylcyclohexyl)carbonyl](1-methylethyl)amino]-2-thiophenecarboxylic acid

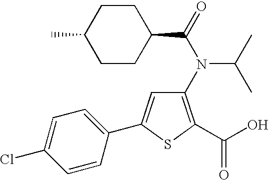

A solution of lithium hydroxide monohydrate (178 mg) in water (6 mL) was added slowly to a solution of Intermediate 23 (307 mg) in THF (2 mL)/MeOH (12 mL) and the mixture left to stir at room temperature for 24 h. The solvents were evaporated, water (40 mL) added and washed with EtOAc (2×20 mL). The aqueous was acidified with 2N HCl to pH 1.0 and extracted with EtOAc (3×20 mL). The first EtOAc extract was partitioned with 2N HCl (10 mL) and the aqueous extracted further with EtOAc (2×20 mL). The organic fractions were dried (Na₂SO₄) and evaporated to give the title compound.

MS calcd for (C₂₂H₂₆ClNO₃S+H)⁺: 420

MS found (electrospray): (M+H)⁺=422

¹H NMR (DMSO): δ 7.85 (2H, d), 7.60 (1H, s), 7.54 (2H, d), 4.74 (1H, m), 1.9-2 (1H, m), 1.65-1.20 (7H, m), 1.13 (3H, d), 0.89 (3H, d), 0.74 (3H, d), 0.7-0.48 (2H, m), carboxylic acid proton not seen.

### Compound C

### 5-(4′-Chloro-4-biphenylyl)-3-[[(trans-4-methylcyclohexyl)carbonyl](1-methylethyl)amino]-2-thiophenecarboxylic acid

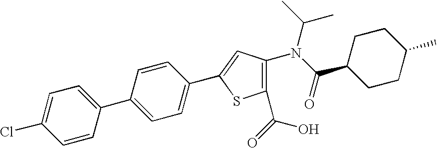

Intermediate 15 (145 mg) was dissolved in THF (0.7 mL) and methanol (0.7 mL). 2N Sodium hydroxide solution (0.7 mL) was added and the mixture stirred at room temperature for 20 h. The methanol and THF were evaporated under vacuum, and the residue was partitioned between DCM and 2N HCl solution. The DCM layer was separated using a hydrophobic frit and concentrated. The residue was purified by reverse phase HPLC on a C8 column using two solvent-gradient elution with (A) acetonitrile containing TFA (0.05%), in (B) water containing TFA (0.1%), from 65% to 85%, with analysis of the fractions by electrospray mass spectroscopy, to give the title compound.

MS calcd for (C₂₈H₃₀ClNO₃S+H)⁺: 496/498

MS found (electrospray): (M+H)⁺=496/498

### Compound D

### 5-(4-Cyanophenyl)-3-[[(trans-4-methylcyclohexyl)carbonyl](1-methylethyl)amino]-2-thiophenecarboxylic acid

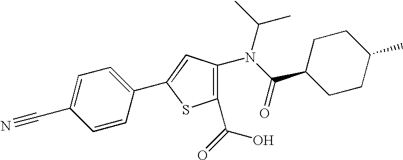

Intermediate 16 (113 mg) was dissolved in THF (0.7 mL) and methanol (0.7 mL). 2N Sodium hydroxide solution (0.7 mL) was added and the mixture stirred at room temperature for 20 h. The methanol and THF were evaporated under vacuum, and the residue was partitioned between DCM and 2N HCl solution. The DCM layer was separated using a hydrophobic frit and concentrated. The residue was purified by ISCO Companion chromatography over silica, eluting with a gradient of 0-30% EtOAc in cyclohexane (containing 0.8% of acetic acid) to give the title compound.

MS calcd for (C₂₃H₂₆N₂O₃S+H)⁺: 411

MS found (electrospray): (M+H)⁺=411

### Compound E

### 5-(4-Acetylphenyl)-3-[[(trans-4-methylcyclohexyl)carbonyl](1-methylethyl)amino]-2-thiophenecarboxylic acid

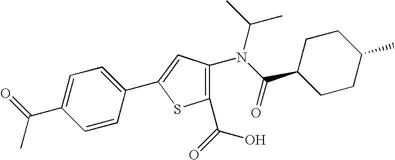

Intermediate 17 (129 mg) was dissolved in THF (0.7 mL) and methanol (0.7 mL). 2N Sodium hydroxide solution (0.7 mL) was added and the mixture stirred at room temperature for 20 h. The methanol and THF were evaporated under vacuum, and the residue was partitioned between DCM and 2N HCl solution. The DCM layer was separated using a hydrophobic frit and concentrated to give the title compound.

MS calcd for (C₂₄H₂₉NO₄S+H)⁺: 428

MS found (electrospray): (M+H)⁺=428

### Compound F

### 3-[[(2,4-Dichlorophenyl)carbonyl](1-methylethyl)amino]-5-[4-(1H-tetrazol-5-yl)phenyl]-2-thiophenecarboxylic acid

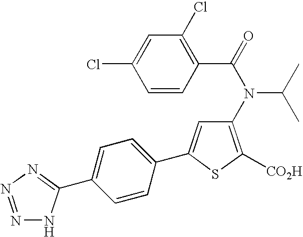

2N NaOH (1 mL) was added to a solution of Intermediate 126 (50 mg, 0.09 mmol) in THF (1 mL)/MeOH (2 mL). The solution was left to stir for 24 h, then evaporated and the residue acidified to pH 1.0 with 2N HCl. The resulting suspension was applied to a 1 g OASIS cartridge and eluted with water (3× column volumes) then MeOH (3× column volumes). The appropriate MeOH fractions were combined and evaporated to give a solid which was purified further by MDAP to give the title compound.

MS calcd for (C₂₂H₁₇Cl₂N₅O₃S+H)⁺: 502/504/506

MS found (electrospray): (M+H)⁺=502/504/506

¹H NMR (DMSO-d₆) δ 8.15-7.96 (dd 4H), 7.8 (s, 1H), 7.7-7.3 (m, 3H), 4.87 (1H, m), 1.4-0.9 (dd, 6H): the carboxylic acid and tetrazole protons are assumed to be exchanged with moisture in the solvent.

The compounds according to the invention may be formulated for administration in any convenient way, and the invention therefore also includes within its scope pharmaceutical compositions for use in therapy, comprising a compound of formula (I) or a pharmaceutically acceptable salt or solvate thereof in admixture with one or more pharmaceutically acceptable diluents or carriers.

The compounds of the present invention can be administered by different routes including intravenous, intraperitoneal, subcutaneous, intramuscular, oral, topical, transdermal, or transmucosal administration. For systemic administration, oral administration is preferred.

For oral administration, for example, the compounds can be formulated into conventional oral dosage forms such as capsules, tablets and liquid preparations such as syrups, elixirs and concentrated drops.

Alternatively, injection (parenteral administration) may be used, e.g., intramuscular, intravenous, intraperitoneal, and subcutaneous. For injection, the compounds of the invention are formulated in liquid solutions, preferably, in pharmaceutically compatible buffers or solutions, such as saline solution, Hank's solution, or Ringer's solution. In addition, the compounds may be formulated in solid form and redissolved or suspended immediately prior to use. Lyophilized forms can also be produced.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, bile salts and fusidic acid derivatives.

In addition, detergents may be used to facilitate permeation. Transmucosal administration, for example, may be through nasal sprays, rectal suppositories, or vaginal suppositories.

For topical administration, the compounds of the invention can be formulated into ointments, salves, gels, or creams, as is generally known in the art.

The amounts of various compounds to be administered can be determined by standard procedures taking into account factors such as the compound (IC₅₀) potency, (EC₅₀) efficacy, and the biological half-life (of the compound), the age, size and weight of the patient, and the disease or disorder associated with the patient. The importance of these and other factors to be considered are known to those of ordinary skill in the art.

Amounts administered also depend on the routes of administration and the degree of oral bioavailability. For example, for compounds with low oral bioavailability, relatively higher doses will have to be administered. Oral administration is a preferred method of administration of the present compounds.

Preferably the composition is in unit dosage form. For oral application, for example, a tablet, or capsule may be administered, for nasal application, a metered aerosol dose may be administered, for transdermal application, a topical formulation or patch may be administered and for transmucosal delivery, a buccal patch may be administered. In each case, dosing is such that the patient may administer a single dose.

Each dosage unit for oral administration contains suitably from 0.01 to 500 mg/Kg, and preferably from 0.1 to 50 mg/Kg, of a compound of Formula (I) or a pharmaceutically acceptable salt thereof, calculated as the free base. The daily dosage for parenteral, nasal, oral inhalation, transmucosal or transdermal routes contains suitably from 0.01 mg to 100 mg/Kg, of a compound of Formula (I). A topical formulation contains suitably 0.01 to 5.0% of a compound of Formula (I). The active ingredient may be administered from 1 to 6 times per day, preferably once, sufficient to exhibit the desired activity, as is readily apparent to one skilled in the art.

Compounds of Formula (I) which are active when given orally can be formulated as syrups, tablets, capsules and lozenges. A syrup formulation will generally consist of a suspension or solution of the compound or salt in a liquid carrier for example, ethanol, peanut oil, olive oil, glycerine or water with a flavoring or coloring agent. Where the composition is in the form of a tablet, any pharmaceutical carrier routinely used for preparing solid formulations may be used. Examples of such carriers include magnesium stearate, terra alba, talc, gelatin, acacia, stearic acid, starch, lactose and sucrose. Where the composition is in the form of a capsule, any routine encapsulation is suitable, for example using the aforementioned carriers in a hard gelatin capsule shell. Where the composition is in the form of a soft gelatin shell capsule any pharmaceutical carrier routinely used for preparing dispersions or suspensions may be considered, for example aqueous gums, celluloses, silicates or oils, and are incorporated in a soft gelatin capsule shell.

Typical parenteral compositions consist of a solution or suspension of a compound or salt in a sterile aqueous or non-aqueous carrier optionally containing a parenterally acceptable oil, for example polyethylene glycol, polyvinylpyrrolidone, lecithin, arachis oil or sesame oil.

Typical compositions for inhalation are in the form of a solution, suspension or emulsion that may be administered as a dry powder or in the form of an aerosol using a conventional non-CFC propellant such as 1,1,1,2-tetrafluoroethane or 1,1,1,2,3,3,3-heptafluoropropane.

A typical suppository formulation comprises a compound of Formula (I) or a pharmaceutically acceptable salt thereof which is active when administered in this way, with a binding and/or lubricating agent, for example polymeric glycols, gelatins, cocoa-butter or other low melting vegetable waxes or fats or their synthetic analogs.

Typical dermal and transdermal formulations comprise a conventional aqueous or non-aqueous vehicle, for example a cream, ointment, lotion or paste or are in the form of a medicated plaster, patch or membrane.

No unacceptable toxicological effects are expected when compounds of the present invention are administered in accordance with the present invention.

### Assays

The potential for compounds of the invention to inhibit NS5B wildtype HCV polymerase activity, genotype 1b, may be demonstrated, for example, using the following in vitro assay:

### In Vitro Detection of Inhibitors of HCV RNA-Dependent RNA Polymerase Activity

Incorporation of [³³P]-GMP into RNA was followed by absorption of the biotin labelled RNA polymer by streptavidin containing SPA beads. A synthetic template consisting of biotinylated 13mer-oligoG hybridised to polyrC was used as a homopolymer substrate.

### Genotype 1b Full-Length Enzyme

Reaction Conditions were 0.5 μM [³³P]-GTP (20 Ci/mMol), 1 mM Dithiothreitol, 20 mM MgCl₂, 5 mM MnCl₂, 20 mM Tris-HCl, pH7.5, 1.6 μg/mL polyC/0.256 μM biotinylated oligoG13, 10% glycerol, 0.01% NP-40, 0.2 u/μL RNasin and 50 mM NaCl.

HCV RNA Polymerase (Recombinant full-length NS5B (Lohmann et al, J. Virol. 71 (11), 1997, 8416. ‘Biochemical properties of hepatitis C virus NS5B RNA-dependent RNA polymerase and identification of amino acid sequence motifs essential for enzymatic activity’) expressed in baculovirus and purified to homogeneity) was added to 4 nM final concentration.

5× concentrated assay buffer mix was prepared using 1M MnCl₂ (0.25 mL), glycerol (2.5 mL), 10% NP-40 (0.025 mL) and Water (7.225 mL), Total 10 mL.

2× concentrated enzyme buffer contained 1M-Tris-HCl, pH7.5 (0.4 mL), 5M NaCl (0.2 mL), 1M-MgCl₂ (0.4 mL), glycerol (1 mL), 10% NP-40 (10 μL), 1M DTT (20 μL) and water (7.97 mL), Total 10 mL.

Substrate Mix was prepared using 5× Concentrated assay Buffer mix (4 μL), [³³P]-GTP (10 μCi/μL, 0.02 μL), 25 μM GTP (0.4 μL), 40 u/1L RNasin (0.1 μL), 20 μg/mL polyrC/biotinylated-oligorG (1.6 μL), and Water (3.94 μL), Total 10 μL.

Enzyme Mix was prepared by adding 1 mg/ml full-length NS5B polymerase (1.5 μL) to 2.81 mL 2×-concentrated enzyme buffer.

The Assay was set up using compound (1 μL), Substrate Mix (10 μL), and Enzyme Mix (added last to start reaction) (10 μL), Total 21 μL.

The reaction was performed in a U-bottomed, white, 96-well plate. The reaction was mixed on a plate-shaker, after addition of the Enzyme, and incubated for 1 h at 22° C. After this time, the reaction was stopped by addition of 40 μL 1.875 mg/ml streptavidin SPA beads in 0.1 M EDTA. The beads were incubated with the reaction mixture for 1 h at 22° C. after which 120 μL 0.1 M EDTA in PBS was added. The plate was sealed, mixed centrifuged and incorporated radioactivity determined by counting in a Trilux (Wallac) or Topcount (Packard) Scintillation Counter.

After subtraction of background levels without enzyme, any reduction in the amount of radioactivity incorporated in the presence of a compound, compared to that in the absence, was taken as a measure of the level of inhibition. Ten concentrations of compounds were tested in three- or fivefold dilutions. From the counts, percentage of inhibition at highest concentration tested or IC₅₀s for the compounds were calculated using GraFit 3, GraFit 4 or GraFit 5 software packages or a data evaluation macro for Excel based on XLFit Software (IDBS).

The potential for compounds of the invention to inhibit HCV replication, genotype 1a and genotype 1b, may be demonstrated, for example, using the following cell based assay:

### Replicon ELISA Cell Based Assay

### Method

100 μL of medium containing 10% FCS were added to each well of clear, flat-bottomed 96 well microplates, excepting wells in the top row. Test compound was diluted in assay medium to twice the final required starting concentration from a 40 mM stock solution in DMSO. 200 μL of the starting dilution were introduced into two wells each in the top row and doubling dilutions made down the plate by the sequential transfer of 100 μL aliquots with thorough mixing in the wells; the final 100 μL were discarded. The two bottom rows were not used for compound dilutions. Huh-7 HCV replicon cell monolayers nearing confluency were stripped from growth flasks with versene-trypsin solution and the cells were resuspended in assay medium at either 2×10⁵ cells/mL (sub-line 5-15; genotype 1b; Lohmann, V., Korner, F., Koch, J-O., Herian, U., Thielmann, L. and Bartenschlager, R., Science, 1999, 285, 110-113) or at 3×10⁵ cells/mL (genotype 1a; Gu, B., Gates, A. T., Isken, O., Behrens, S. E. and Sarisky, R. T., J. Virol., 2003, 77, 5352-5359). 100 μL of cell suspension were added to all wells and the plates incubated at 37° C. for 72 hours in a 5% CO₂ atmosphere.

Following incubation, the assay medium was aspirated from the plates. The cell sheets were washed by gentle immersion in phosphate buffered saline (PBS), which was then aspirated off, and fixed with acetone:methanol (1:1) for 5 minutes. Following a further wash with PBS, 100 μL of ELISA diluent (PBS+0.05% v/v Tween 20+2% w/v skimmed milk powder) were added to all wells and the plates incubated at 37° C. for 30 minutes on an orbital platform. The diluent was removed and each well then received 50 μL of a 1/200 dilution of anti-HCV specific, murine, monoclonal antibody (either Virostat #1872 or #1877), except for wells in one of the compound-free control rows which received diluent alone to act as negative controls. The plates were incubated at 37° C. for 2 hours and washed 3 times with PBS/0.05% Tween 20, then 50 μL of horseradish peroxidase conjugated, anti-mouse, rabbit polyclonal serum (Dako #P0260), diluted 1/1000, were added to all wells. The plates were incubated for a further hour, the antibody removed and the cell sheets washed 5 times with PBS/Tween and blotted dry. The assay was developed by the addition of 50 μL of ortho-phenylenediamine/peroxidase substrate in urea/citrate buffer (SigmaFast, Sigma #P-9187) to each well, and colour allowed to develop for up to 15 minutes. The reaction was stopped by the addition of 25 μL per well of 2 M sulphuric acid and the plates were read at 490 nm on a Fluostar Optima spectrophotometer.

The substrate solution was removed and the plates were washed in tap water, blotted dry and the cells stained with 5% carbol fuchsin in water for 30 minutes. The stain was discarded and the cell sheets washed, dried and examined microscopically to assess cytotoxicity.

### Data Analysis

The absorbance values from all compound-free wells that had received both primary and secondary antibodies were averaged to obtain a positive control value. The mean absorbance value from the compound-free wells that had not received the primary antibody was used to provide the negative (background) control value. The readings from the duplicate wells at each compound concentration were averaged and, after the subtraction of the mean background from all values, were expressed as a percentage of the positive control signal. The quantifiable and specific reduction of expressed protein detected by the ELISA in the presence of a drug can be used as a measure of replicon inhibition. GraFit software (Erithacus Software Ltd.) was used to plot the curve of percentage inhibition against compound concentration and derive the 50% inhibitory concentration (IC₅₀) for the compound.

### Results

All compounds were assayed at least twice and the mean data are included in the table below.

| | IC₅₀ in 1a | IC₅₀ in 1b |
|---|---|---|
| | replicon | replicon cell- |
| | cell-based assay | based assay |
| Compound | (μM) | (μM) |
| Example 1 | ++++++ | ++++++ |
| Example 2 | ++++++ | ++++++ |
| Example 3 | +++++ | ++++++ |
| Example 4 | +++++ | ++++++ |
| Example 5 | ++++++++ | ++++++++ |
| Example 6 | +++++ | ++++++ |
| Example 7 | ++++++++ | ++++++++ |
| Example 8 | ++++++ | ++++++ |
| Example 9 | ++++ | +++++ |
| Example 10 | +++++ | ++++ |
| Example 11 | ++++ | ++++ |
| Example 12 | ++++ | +++++ |
| Example 13 | ++++ | ++++ |
| Example 14 | ++++ | +++ |
| Example 15 | ++++ | +++++ |
| Example 16 | ++++++ | ++++++ |
| Example 17 | ++++ | ++++ |
| Example 18 | +++++ | ++++++ |
| Example 19 | ++++++++ | ++++++++ |
| Example 20 | ++++ | ++++++ |
| Example 21 | +++++ | ++++ |
| Example 22 | +++++ | +++++ |
| Example 23 | +++++ | ++++++ |
| Example 24 | ++++ | ++++++ |
| Example 25 | ++++ | ++++ |
| Example 26 | ++++ | +++ |
| Example 27 | ++++ | ++++ |
| Example 28 | ++++ | ++++ |
| Example 29 | ++++ | ++++ |
| Example 30 | ++++ | +++ |
| Example 31 | ++++ | ++++ |
| Example 32 | ++++ | +++++ |
| Example 33 | ++++++++ | ++++++++ |
| Example 34 | ++++++ | +++++ |
| Example 35 | ++++++ | +++++++ |
| Example 36 | ++++++ | ++++++ |
| Example 39 | ++++++ | +++++++ |
| Example 40 | ++++ | +++++++ |
| Example 41 | ++++ | +++++ |
| Example 42 | ++++ | ++++++ |
| Example 43 | +++ | +++++ |
| Example 46 | ++++ | ++++ |
| Compound A | ++ | ++++ |
| Compound B | ++ | ++ |
| Compound C | ++ | + |
| Compound D | ++ | ++ |
| Compound E | ++ | ++ |
| Compound F | ++ | + |

Examples 37 and 38 are salts of the compound of Example 2. Examples 44 and 45 are salts of the compound of Example 1.

### Activity Ranges

| | |
|---|---|
| Genotype 1a | Genotype 1b |

| | | | | | |
|---|---|---|---|---|---|
| + | >5.00 | μM | + | >5.00 | μM |
| ++ | 1.00-5.00 | μM | ++ | 1.00-5.00 | μM |
| +++ | 0.50-1.00 | μM | +++ | 0.50-1.00 | μM |
| ++++ | 0.10-0.50 | μM | ++++ | 0.10-0.50 | μM |
| +++++ | 0.05-0.10 | μM | +++++ | 0.05-0.10 | μM |
| ++++++ | 0.01-0.05 | μM | ++++++ | 0.01-0.05 | μM |
| +++++++ | 0.005-0.01 | μM | +++++++ | 0.005-0.01 | μM |
| ++++++++ | <0.005 | μM | ++++++++ | <0.005 | μM |

Compound A corresponds to the compound disclosed as Example 317 in WO2002/100851, 3-[isopropyl-(4-methylcyclohexanecarbonyl)-amino]-5-phenyl-thiophene-2-carboxylic acid.

Compound B corresponds to the compound disclosed as Example 570 in WO2002/100851, 5-(4-chlorophenyl)-3-[isopropyl-(4-methylcyclohexanecarbonyl)amino]-thiophene-2-carboxylic acid.

Compound C corresponds to the compound disclosed as Example 571 in WO2002/100851, 5-(4′-chloro-biphenyl-4-yl)-3-[isopropyl-(4-methylcyclohexanecarbonyl)amino]-thiophene-2-carboxylic acid.

Compound D corresponds to the compound disclosed as Example 576 in WO2002/100851, 5-(4-cyanophenyl)-3-[isopropyl-(4-methylcyclohexanecarbonyl)amino]-thiophene-2-carboxylic acid.

Compound E corresponds to the compound disclosed as Example 460 in WO2002/100851, 5-(4-acetylphenyl)-3-[isopropyl-(4-methylcyclohexanecarbonyl)amino]-thiophene-2-carboxylic acid.

Compound F corresponds to the compound disclosed as Example 430 in WO2002/100851, 3-[[(2,4-Dichlorophenyl)carbonyl](1-methylethyl)amino]-5-[4-(1H-tetrazol-5-yl)phenyl]-2-thiophenecarboxylic acid

Compounds A to F may be made according to the processes described in WO2002/100851 or as described hereinabove.

Structures of Compounds A-F are shown below for the avoidance of doubt.

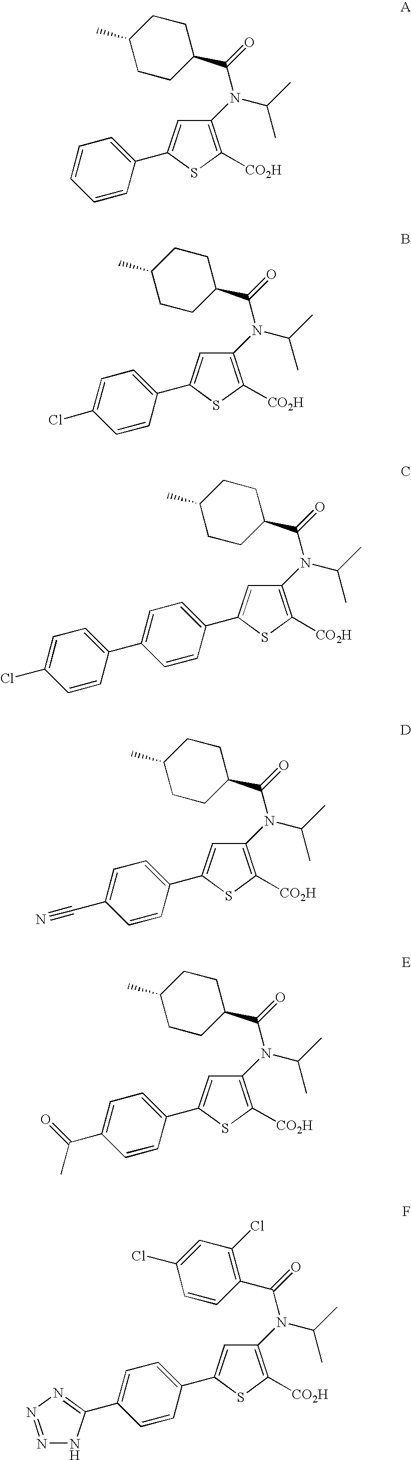

The compounds of the present invention which have been tested demonstrate a surprisingly superior potency as HCV polymerase inhibitors, as shown by the IC₅₀ values in the cell-based assays across both of the 1a and 1b genotypes of HCV, compared to Compounds A-F. Accordingly, the compounds of the present invention are of great potential therapeutic benefit in the treatment and prophylaxis of HCV.

The pharmaceutical compositions according to the invention may also be used in combination with other therapeutic agents, for example immune therapies (eg. Interferon, such as Interferon alfa-2a (Roferon-A; Hoffmann-La Roche), inteferon alpha-2b (Intron-A; Schering-Plough), interferon alfacon-1 (Infergen; Intermune), peginterferon alpha-2b (Peg-Intron; Schering-Plough) or peginterferon alpha-2a (Pegasys; Hoffmann-La Roche)), therapeutic vaccines, antifibrotic agents, anti-inflammatory agents such as corticosteroids or NSAIDs, bronchodilators such as beta-2 adrenergic agonists and xanthines (e.g. theophylline), mucolytic agents, anti-muscarinics, anti-leukotrienes, inhibitors of cell adhesion (e.g. ICAM antagonists), anti-oxidants (eg N-acetylcysteine), cytokine agonists, cytokine antagonists, lung surfactants and/or antimicrobial, anti-viral agents (eg ribavirin and amantidine), and anti-HCV agents (eg HCV NS3 protease inhibitors or HCV NS5b polymerase inhibitors). The compositions according to the invention may also be used in combination with gene replacement therapy.

The invention thus provides, in a further aspect, a combination comprising a compound of Formula (I) together with at least one other therapeutically active agent, especially Interferon, ribavirin and/or an additional anti-HCV agent.

The combinations referred to above may conveniently be presented for use in the form of a pharmaceutical formulation and thus pharmaceutical formulations comprising a combination as defined above together with a pharmaceutically acceptable carrier thereof represent a further aspect of the invention.

The individual components of such combinations may be administered either sequentially or simultaneously in separate or combined pharmaceutical formulations. Appropriate doses of known therapeutic agents will be readily appreciated by those skilled in the art.

All publications, including but not limited to patents and patent applications cited in this specification are herein incorporated by reference as if each individual publication were specifically and individually indicated to be incorporated by reference as though fully set forth.

What is claimed is:

1. A compound of Formula (I): 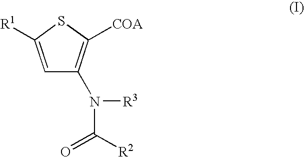
wherein:
A represents hydroxy;
R¹ represents —R^{X}—R^{Y};
R^{X} represents phenyl (optionally substituted by halo, methyl, ethyl, methoxy or trifluoromethyl) or 5 or 6-membered heteroaryl bonded through a ring carbon atom to the carbon atom of the thiophene; in the case of 6-membered heteroaryl there not being a ring nitrogen ortho to the attachment point to the thiophene
R^{Y} represents 8, 9 or 10-membered heteroaryl, bonded such that when R^{X} is phenyl, R^{Y} is in the para-position; the heteroaryl not being imidazo[1,2-a]pyridin-6-yl
R² represents C₅₋₇cycloalkyl optionally substituted by one or more substitutents selected from —C₁₋₆alkyl or —OR^{A};
R³ represents linear or branched —C₁₋₆alkyl (unsubstituted), or linear or branched —C₁₋₆alkyl substituted with C₃₋₆cycloalkyl;
R^{A} represents hydrogen or —C₁₋₆alkyl;
or a pharmaceutically acceptable salt, thereof.

2. A compound as claimed in claim 1 which is selected from the group consisting of:
5-(4-Furo[3,2-b]pyridin-2-ylphenyl)-3-[[(trans-4-methylcyclohexyl)carbonyl](1-methylethyl)amino]-2-thiophenecarboxylic acid;
3-[[(trans-4-Methylcyclohexyl)carbonyl](1-methylethyl)amino]-5-(4-pyrazolo[1,5-a]pyrimidin-2-ylphenyl)-2-thiophenecarboxylic acid;
5-(4-Imidazo[1,2-a]pyridin-2-ylphenyl)-3-[[(trans-4-methylcyclohexyl)carbonyl](1-methylethyl)amino]-2-thiophenecarboxylic acid;
5-(4-Imidazo[2,1-b][1,3]thiazol-6-ylphenyl)-3-[[(trans-4-methylcyclohexyl)carbonyl](1-methylethyl)amino]-2-thiophenecarboxylic acid; 5-[4-(7-Amino-5-methylpyrazolo[1,5-a]pyrimidin-2-yl)phenyl]-3-[[(trans-4-methyl-cyclohexyl)carbonyl](1-methylethyl)amino]-2-thiophenecarboxylic acid;
3-[[(trans-4-Methylcyclohexyl)carbonyl](1-methylethyl)amino]-5[4-(5-methylpyrazolo-[1,5-a]pyrimidin-2-yl)phenyl]-2-thiophenecarboxylic acid;
5-[4-(7-Aminopyrazolo[1,5-a]pyrimidin-2-yl)phenyl]-3-[[(trans-4-methylcyclohexyl)-carbonyl](1-methylethyl)amino]-2-thiophenecarboxylic acid;
3-[[(4-Methylcyclohexyl)carbonyl](1-methylethyl)amino]-5-(4-[1,3]oxazolo[4,5-b]pyridin-2-ylphenyl)-2-thiophenecarboxylic acid;
5-(4-furo[2,3-b]pyridin-5-ylphenyl)-3-[[(trans-4-methylcyclohexyl)carbonyl](1-methylethyl)amino]-2-thiophenecarboxylic acid;
5-[4-(5-Amino-1,3-benzoxazol-2-yl)phenyl]-3-[[(trans-4-methylcyclohexyl)carbonyl](1-methylethyl)amino]-2-thiophenecarboxylic acid;
3-[[(4-Methylcyclohexyl)carbonyl](1-methylethyl)amino]-5-(4-[1,3]oxazolo[5,4-b]pyridin-2-ylphenyl)-2-thiophenecarboxylic acid;
5-(4-Furo[3,2-c]pyridin-2-ylphenyl)-3-[[(trans-4-methylcyclohexyl)carbonyl](1-methylethyl)-amino]-2-thiophenecarboxylic acid;
5-(4-Imidazo[1,2-a]pyridin-2-ylphenyl)-3-((1-methylethyl){[trans-4-(trifluoromethyl)cyclohexyl]carbonyl}amino)-2-thiophenecarboxylic acid;
3-((1-Methylethyl){[trans-4-(trifluoromethyl)cyclohexyl]carbonyl}amino)-5-(4-pyrazolo[1,5-a]pyrimidin-2-ylphenyl)-2-thiophenecarboxylic acid;
5-[4-(4-Amino-1,3-benzoxazol-2-yl)phenyl]-3-[[(trans-4-methylcyclohexyl)carbonyl](1-methylethyl)amino]-2-thiophenecarboxylic acid;
3-[[(trans-4-Methylcyclohexyl)carbonyl](1-methylethyl)amino]-5-(4-pyrazolo[1,5-a]pyrimidin-5-ylphenyl)-2-thiophenecarboxylic acid;
5-(6-Furo[3,2-b]pyridin-2-yl-3-pyridinyl)-3-[[(trans-4-methylcyclohexyl)carbonyl](1-methylethyl)amino]-2-thiophenecarboxylic acid;
5-[4-(7-Hydroxy-1-benzofuran-2-yl)phenyl]-3-[[(trans-4-methylcyclohexyl)carbonyl](1-methylethyl)amino]-2-thiophenecarboxylic acid;
5-[4-(7-Hydroxy-1,3-benzoxazol-2-yl)phenyl]-3-[[(trans-4-methylcyclohexyl)carbonyl](1-methylethyl)amino]-2-thiophenecarboxylic acid;
5-(4-Imidazo[2,1-b][1,3]thiazol-6-ylphenyl)-3-((1-methylethyl){[trans-4-(trifluoromethyl)cyclohexyl]carbonyl}amino)-2-thiophenecarboxylic acid;
5-(4-Furo[3,2-b]pyridin-2-ylphenyl)-3-((1-methylethyl){[trans-4-(trifluoromethyl)cyclohexyl]carbonyl}amino)-2-thiophenecarboxylic acid;
5-[4-(6-Amino-1,3-benzoxazol-2-yl)phenyl]-3-[[(trans-4-methylcyclohexyl)carbonyl](1-methylethyl)amino]-2-thiophenecarboxylic acid;
3-[[(trans-4-Methylcyclohexyl)carbonyl](1-methylethyl)amino]-5-(4-pyrazolo[1,5-b]pyridazin-2-ylphenyl)-2-thiophenecarboxylic acid;
3-((1-Methylethyl){[trans-4-(trifluoromethyl)cyclohexyl]carbonyl}amino)-5-(4-[1,3]oxazolo[4,5-b]pyridin-2-ylphenyl)-2-thiophenecarboxylic acid;
3-((1-Methylethyl){[trans-4-(trifluoromethyl)cyclohexyl]carbonyl}amino)-5-[4-(5-methylpyrazolo[1,5-a]pyrimidin-2-yl)phenyl]-2-thiophenecarboxylic acid;
5-[4-(6-Aminoimidazo[1,2-a]pyridin-2-yl)phenyl]-3-[[(trans-4-methylcyclohexyl)carbonyl](1-methylethyl)amino]-2-thiophenecarboxylic acid;
5-[4-(1H-Benzimidazol-5-yl)phenyl]-3-[[(trans-4-methylcyclohexyl)carbonyl](1-methylethyl)amino]-2-thiophenecarboxylic acid;
5-[4-(5-Amino-1-benzofuran-2-yl)phenyl]-3-[[(trans-4-methylcyclohexyl)carbonyl](1-methylethyl)amino]-2-thiophenecarboxylic acid;
5-[4-(6-Amino-1-benzofuran-2-yl)phenyl]-3-[[(trans-4-methylcyclohexyl)carbonyl](1-methylethyl)amino]-2-thiophenecarboxylic acid;
5-[5-(1,3-Benzoxazol-2-yl)-3-furanyl]-3-[[(trans-4-methylcyclohexyl)carbonyl](1-methylethyl)amino]-2-thiophenecarboxylic acid;
5-(6-Imidazo[2,1-b][1,3]thiazol-6-yl-3-pyridinyl)-3-[[(trans-4-methylcyclohexyl)carbonyl](1-methylethyl)amino]-2-thiophenecarboxylic acid;
5-[4-(7-Aminopyrazolo[1,5-a]pyrimidin-2-yl)-3-chlorophenyl]-3-[[(trans-4-methylcyclohexyl)carbonyl](1-methylethyl)amino]-2-thiophenecarboxylic acid;
3-[[(trans-4-Methylcyclohexyl)carbonyl](1-methylethyl)amino]-5-(6-pyrazolo[1,5-a]pyrimidin-2-yl-3-pyridinyl)-2-thiophenecarboxylic acid;
3-{(Cyclopropylmethyl)[(trans-4-methylcyclohexyl)carbonyl]amino}-5-(4-pyrazolo[1,5-a]pyrimidin-2-ylphenyl)-2-thiophenecarboxylic acid;
4-[[(trans-4-Methylcyclohexyl)carbonyl](1-methylethyl)amino]-5′-pyrazolo[1,5-a]pyrimidin-2-yl-2,2′-bithiophene-5-carboxylic acid;
3-[{[(1S,2R,4S)-2-Hydroxy-4-methylcyclohexyl]carbonyl}(1-methylethyl)amino]-5-(4-pyrazolo[1,5-a]pyrimidin-2-ylphenyl)-2-thiophenecarboxylic acid;
5-[4-(1H-Indol-5-yl)phenyl]-3-[[(trans-4-methylcyclohexyl)carbonyl](1-methylethyl)amino]-2-thiophenecarboxylic acid;
5-[4-(1H-Indol-6-yl)phenyl]-3-[[(trans-4-methylcyclohexyl)carbonyl](1-methylethyl)amino]-2-thiophenecarboxylic acid;
5′-Furo[3,2-b]pyridin-2-yl-4-[[(trans-4-methylcyclohexyl)carbonyl](1-methylethyl)amino]-2,2′-bithiophene-5-carboxylic acid;
3-[[(trans-4-Methylcyclohexyl)carbonyl] (1-methylethyl)amino]-5-(6-[1,3]oxazolo[4,5-b]pyridin-2-yl-3-pyridinyl)-2-thiophenecarboxylic acid;
Sodium 5-(4-furo[3,2-c]pyridin-2-ylphenyl)-3-[[(trans-4-methylcyclohexyl)carbonyl](1-methylethyl)amino]-2-thiophenecarboxylate;
Sodium 3-[[(trans-4-methylcyclohexyl)carbonyl](1-methylethyl)amino]-5-(4-pyrazolo[1,5-a]pyrimidin-2-ylphenyl)-2-thiophenecarboxylate;
Ammonium 3-[[(trans-4-methylcyclohexyl)carbonyl](1-methylethyl)amino]-5-(4-pyrazolo[1,5-a]pyrimidin-2-ylphenyl)-2-thiophenecarboxylate;
Ammonium 3-{ethyl[(trans-4-methylcyclohexyl)carbonyl]amino}-5-(4-pyrazolo[1,5-a]pyrimidin-2-ylphenyl)-2-thiophenecarboxylate;
Potassium 5-(4-furo[3,2-b]pyridin-2-ylphenyl)-3-[[(trans-4-methylcyclohexyl)carbonyl](1-methylethyl)amino]-2-thiophenecarboxylate;
Trisamine 5-(4-furo[3,2-b]pyridin-2-ylphenyl)-3-[[(trans-4-methylcyclohexyl)carbonyl](1-methylethyl)amino]-2-thiophenecarboxylate;
pharmaceutically acceptable salts thereof, enantiomers, and pharmaceutically acceptable salts of enantiomers thereof where appropriate.

3. A compound as claimed in claim 1 wherein R^{X} represents phenyl optionally substituted by halo, methyl, methoxy or trifluoromethyl.

4. A compound as claimed in claim 1, wherein R^{Y} represents furo[3,2-b]pyridin-2-yl, pyrazolo[1,5-a]pyrimidin-2-yl, imidazo[1,2-a]pyridin-2-yl, 4-imidazo[2,1-b][1,3]thiazol-6-yl, 7-amino-5-methylpyrazolo[1,5-a]pyrimidin-2-yl, 5-methylpyrazolo-[1,5-a]pyrimidin-2-yl, 7-aminopyrazolo[1,5-a]pyrimidin-2-yl, [1,3]oxazolo[4,5-b]pyridin-2-yl, 4-furo[2,3-b]pyridin-5-yl, 5-amino-1,3-benzoxazol-2-yl, [1,3]oxazolo[5,4-b]pyridin-2-yl, or 4-furo[3,2-c]pyridin-2-yl.

5. A compound as claimed in claim 1 wherein R² represents trans-4-methylcyclohexyl.

6. A compound as claimed in claim 1 wherein R³ represents 1-methylethyl.

7. A compound as claimed in claim 1 wherein R^{x} represents unsubstituted phenyl, unsubstituted thienyl attached to the rest of the molecule via its 2- and 5-positions, unsubstituted furanyl attached to the thiophene via its 3-position and to R^{y} via its 5-position, or unsubstituted pyridinyl (wherein the ring nitrogen is not in the ortho position relative to the thiophene attachment) attached to the thiophene via its 5-position and to R^{y} via its 2-position; R^{y} represents furo[3,2-b]pyridin-2-yl, pyrazolo[1,5-a]pyrimidin-2-yl, imidazo[1,2-a]pyridin-2-yl, imidazo[2,1-b][1,3]thiazol-6-yl, 7-amino-5-methylpyrazolo[1,5-a]pyrimidin-2-yl, 5-methylpyrazolo-[1,5-a]pyrimidin-2-yl, 7-aminopyrazolo[1,5-a]pyrimidin-2-yl, [1,3]oxazolo[4,5-b]pyridin-2-yl, furo[2,3-b]pyridin-5-yl, 5-amino-1,3-benzoxazol-2-yl, [1,3]oxazolo[5,4-b]pyridin-2-yl, furo[3,2-c]pyridin-2-yl, 4-amino-1,3-benzoxazol-2-yl, pyrazolo[1,5-a]pyrimidin-5-yl, 7-hydroxy-1-benzofuran-2-yl, 7-hydroxy-1,3-benzoxazol-2-yl, pyrazolo[1,5-b]pyridazin-2-yl, 6-aminoimidazo[1,2-a]pyridin-2-yl, 1H-benzimidazol-5-yl, 5-amino-1-benzofuran-2-yl, 6-amino-1-benzofuran-2-yl, 6-amino-1,3-benzoxazol-2-yl, 1,3-benzoxazol-2-yl, 1H-indol-5-yl or 1H-indol-6-yl; R² represents trans-4-methylcyclohexyl or trans-4-trifluoromethylcyclohexyl; and R³ represents 1-methylethyl, ethyl or cyclopropylmethyl.

8. A compound as claimed in claim 7 wherein R^{x} represents unsubstituted phenyl; R^{y} represents furo[3,2-b]pyridin-2-yl, pyrazolo[1,5-a]pyrimidin-2-yl, imidazo[1,2-a]pyridin-2-yl, 4-imidazo[2,1-b][1,3]thiazol-6-yl, 7-amino-5-methylpyrazolo[1,5-a]pyrimidin-2-yl, 5-methylpyrazolo-[1,5-a]pyrimidin-2-yl, 7-aminopyrazolo[1,5-a]pyrimidin-2-yl, [1,3]oxazolo[4,5-b]pyridin-2-yl, furo[2,3-b]pyridin-5-yl, 5-amino-1,3-benzoxazol-2-yl, [1,3]oxazolo[5,4-b]pyridin-2-yl or furo[3,2-c]pyridin-2-yl; R represents trans-4-methylcyclohexyl; and R³ represents 1-methylethyl.

9. A compound as claimed in claim 8 wherein R^{x} represents unsubstituted phenyl; R^{y} represents furo[3,2-b]pyridin-2-yl, pyrazolo[1,5-a]pyrimidin-2-yl, imidazo[1,2-a]pyridin-2-yl; R² represents trans-4-methylcyclohexyl; and R³ represents 1-methylethyl.

10. A method of treating or preventing viral infection which comprises administering to a human in need thereof, an effective amount of a compound of Formula (I) 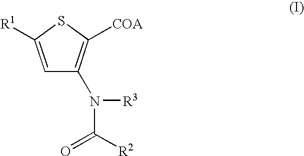
wherein A, R¹, R² and R³ are as defined in claim 1
or a pharmaceutically acceptable salt thereof.

11. A method as claimed in claim 10 wherein the viral infection is an HCV infection.

12. A method as claimed in claim 10 in which the compound is administered in an oral dosage form.

13. (canceled)

14. (canceled)

15. (canceled)

16. A pharmaceutical formulation comprising a compound of Formula (I) according to claim 1 in conjunction with at least one pharmaceutically acceptable diluent or carrier.

17. (canceled)

18. (canceled)

19. A combination comprising a compound of Formula (I) as defined in claim 1, together with at least one other therapeutically active agent.

20. A combination as claimed in claim 19, wherein the other therapeutically active agent is selected from Interferon, ribavirin and/or an additional anti-HCV agent.
